SEQUENCE LISTING (14 sequences)

SEQ ID NO: 1 - protein, 210 aa, Homo sapiens [sequence omitted]

SEQ ID NO: 2 - DNA, 1212 bp, Homo sapiens
gggtacaggaaagaagccagtgccccaggtcacctaagacaggcatcaagctcattcggcaaaatccagttgggttgttctaactggctcattcttacttgggtgacctctgcatggcaggagtaacttctgtgcctttgttttcatgttcagcgccatggatattgcaccctgggggaagctttcaacagactggacttctcaactgccattctggattccagaagatttaactacgtggtccggctgttggagctgatagcaaagtcacagctcacatccctgagtggcatcgcccaaaagaacttcatgaatattttggaaaaagtggtactgaaagtccttgaagaccagcaaaacattagactaataagggaactactccagaccctctacacatccttatgtacactggtccaaagagtcggcaagtctgtgctggtcgggaacattaacatgtgggtgtatcggatggagacgattctccactggcagcagcagctgaacaacattcagatcaccaggcctgccttcaaaggcctcaccttcactgacctgcctttgtgcctacaactgaacatcatgcagaggctgagcgacgggcgggacctggtcagcctgggccaggctgcccccgacctgcacgtgctcagcgaagaccggctgctgtggaagaaactctgccagtaccacttctccgagcggcagatccgcaaacgattaattctgtcagacaaagggcagctggattggaagaagatgtatttcaaactcgtccgatgttacccaaggaaagagcagtatggagatacccttcagctccgcaaacactgtcacatcctttcctggaagggcactgaccatccgtgcactgccaataacccagagagctgctccgtttcactttcaccccaggactttatcaacttgttcaagttctgaatcccagcacatgacaacacttcagaagggtccccctgctgactggagagctgggaatatggcatttggacacttcatttgtaaatagtgtacattttaaacattggctcgaaacttcagagataagtcatggagaggacattggaggggagaaatgcagttgctgactgggaatttaagaatgtgaacttctcactagaattggtatggaaaagcaaaatactgtaaataaactttttttctaacaatttgccaaaaaaaaaaaaaaaa

SEQ ID NO: 3 - protein, 355 aa, Mus musculus
MPFLGQDWRSPGQSWVKTADGWKRFLDEKSGSFVSDLSSYCNKEVYSKENLFSSLNYDVAAKKRKKDIQNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETILHWQQQLNSIQISRPAFKGLTITDLPVCLQLNIMQRLSDGRDLVSLGQAAPDLHVLSEDRLLWKRLCQYHFSERQIRKRLILSDKGQLDWKKMYFKLVRCYPRREQYGVTLQLCKHCHILSWKGTDHPCTANNPESCSVSLSPQDFINLFKF

SEQ ID NO: 4 - DNA, 1967 bp, Mus musculus
gcgaccttccccaacgcctgcgcccctgtgagtgcaaggatccccgcgcccacccaggatccgcagccctccacactagttgacccactcttgtcccggtcgccgcctgcgtcgttccccagcatcttcccaacgcgccgcataccctgggcaagccaggccggttcctggctgtcaatccgtcccgtccgtcggtcgcgtccgcgctctgtaccatgccgttccttgggcaggactggcggtccccgggccagagctgggtgaagacggcggacggctggaagcgcttcttggatgagaaaagcggcagcttcgtgagcgacctcagcagttactgcaacaaggaggtatacagtaaggagaatctgttcagcagcctgaactacgacgtcgcagccaagaagagaaagaaagacattcagaacagcaaaaccaaaactcagtacttccatcaagaaaagtggatctatgttcacaaaggaagtacgaaggagcgccatggatactgtactttgggggaagctttcaacagactggacttctcgactgccatcctggattccagaagattcaactacgtagtaaggctgttggagctgatagcaaagtcacagctcacatccctgagtggcatcgcccaaaagaacttcatgaacattttggaaaaagtggtactgaaagttcttgaagaccagcaaaacataagacttatacgggaacttctccagactctctacacatccttatgcacactggtgcagagagtcggcaagtctgtgctggtgggcaacattaacatgtgggtgtatcggatggagaccattctacactggcagcagcagctgaatagcatccagatcagcaggcctgccttcaaaggcctcacgatcaccgacctgcctgtgtgcttacaactgaacatcatgcagaggctgagtgacgggcgggacctggtcagcctgggccaggcagccccagacctgcatgtgctcagtgaggaccggctactgtggaagagactctgccagtaccacttctcagagaggcagattcgcaagcgtttgatcttgtctgacaaagggcagctggattggaagaagatgtattttaagcttgtacgatgttacccaagaagagagcagtatggggtcaccctgcagctttgcaaacactgccacattctctcctggaagggcactgaccatccgtgcacggccaacaacccagagagctgctccgtctcactttcccctcaagactttatcaatttgttcaagttctgaataatcccagcacacgacaacacttcagaaggcttctaattggatggctgggagtcgggacacttcatttgtaaatagtgtacattttaagcattggcttgaaactgcgggggatacgtcattgaggagacgttggcggggaagagatgcagttgccgatggaaatttacaaatgtgaattccacatgagaactggtacagaaaagcagaaatactgtaaatagactttttattttccctaacgatttgcaagcaagactataaaggcaagaactctatgtcagccatggaaacggagtcctcttgagttccctaggaagaaaaaggcaaaaagctcaaaaacaagatggaacactctgtttacaatgtgaaaatgttgttaagacaaaaataaggaagaaggaagatgaacgctgtcattgagaaacccttgggctttgggtttggattcggggtttgttttcagcaggccaagaagtatatccacctgaaatctgcacgggcttaagtccttatcctatgaagatgccacacaatggtctacctctaaaagcatagcgtgttctctggcaacatactttatctgggaggcaatgtctgtgtttcatgtaagttctatactctgtgaagtgatctaagatgggaaggctgttaggaaaaaaaaaaataaaaaaaaaaaaaaaa

SEQ ID NO: 5 - DNA, 24 bp, Artificial Sequence
  Synthetic Construct
ctttcaacagactggacttctcga

SEQ ID NO: 6 - DNA, 23 bp, Artificial Sequence
  Synthetic Construct
cagctccaacagccttactacgt

SEQ ID NO: 7 - DNA, 28 bp, Artificial Sequence
  Synthetic Construct
tgccatcctggattccagaagattcaac

SEQ ID NO: 8 - DNA, 25 bp, Artificial Sequence
  Synthetic Construct
ttgtccaagaaacggcattgtcaag

SEQ ID NO: 9 - DNA, 25 bp, Artificial Sequence
  Synthetic Construct
aaagccaccatcatgtacctgtctg

SEQ ID NO: 10 - protein, 356 aa, Danio rerio
MPFLGQDWRSPGQSWVKTEDGWKKTTKDDNETNNNVLERKSYCKEEHDKENLILSINYDVAAKKRKKDLPNNNTKIPYFYKDKWIYVHKGSTKERHGYCTLGEAFNRLDFCSAIKDTRRFNYVVRLLELIAKSQLPSLSGVAQKNYMNILERVVQKVLDDQQNVRPIKELLQTLYASLCSLVQDMGKSVLVGNINIWVHRMENILQWQQQLDNIQINRPKNTGMTLLELPVSLQLNIMQRLSDGRDLVSLGQVCPDLSMLTEDRLLWKKLCQYHFTDRQIRKRLMVSDKGQLEWKKMYFKLCRCYPHKEQYSDTLQFCTHCHILFWKDTDHPCTANNPESCCKAVSPQGFINLFKF

SEQ ID NO: 11 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 12 - DNA, 6318 bp, Homo sapiens
tagtaagacaggtgccttcagttcactctcagtaaggggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacagaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacgcttaaaacctagaacttcaaaatgttcgtattctattcaaaaggaaatatatatatatatatatatatatatatatatatatataaattaaaaaggaaagaaaactaacaaccaaccaaccaaccaaccaaccacaaaccaccctaaaatgacagccgctgatgtctgggcatcagcctttgtactctgtttttttaagaaagtgcagaatcaacttgaagcaagctttctctcataacgtaatgattatatgacaatcctgaagaaaccacaggttccatagaactaatatcctgtctctctctctctctctctctctctcttttttttttctttttccttttgccatggaatctgggtgggagaggatactgcgggcaccagaatgctaaagtttcctaacattttgaagtttctgtagttcatccttaatcctgacacccatgtaaatgtccaaaatgttgatcttccactgcaaatttcaaaagccttgtcaatggtcaagcgtgcagcttgttcagcggttctttctgaggagcggacaccgggttacattactaatgagagttgggtagaactctctgagatgtgttcagatagtgtaattgctacattctctgatgtagttaagtatttacagatgttaaatggagtatttttattttatgtatatactatacaacaatgttcttttttgttacagctatgcactgtaaatgcagccttcttttcaaaactgctaaatttttcttaatcaagaatattcaaatgtaattatgaggtgaaacaattattgtacactaacatatttagaagctgaacttactgcttatatatatttgattgtaaaaacaaaaagacagtgtgtgtgtctgttgagtgcaacaagagcaaaatgatgctttccgcacatccatcccttaggtgagcttcaatctaagcatcttgtcaagaaatatcctagtcccctaaaggtattaaccacttctgcgatatttttccacattttcttgtcgcttgtttttctttgaagttttatacactggatttgttaggggaatgaaattttctcatctaaaatttttctagaagatatcatgattttatgtaaagtctctcaatgggtaaccattaagaaatgtttttattttctctatcaacagtagttttgaaactagaagtcaaaaatctttttaaaatgctgttttgttttaatttttgtgattttaatttgatacaaaatgctgaggtaataattatagtatgatttttacaataattaatgtgtgtctgaagactatctttgaagccagtatttctttcccttggcagagtatgacgatggtatttatctgtattttttacagttatgcatcctgtataaatactgatatttcattcctttgtttactaaagagacatatttatcagttgcagatagcctatttattataaattatgagatgatgaaaataataaagccagtggaaattttctacctaggatgcatgacaattgtcaggttggagtgtaagtgcttcatttgggaaattcagcttttgcagaagcagtgtttctacttgcactagcatggcctctgacgtgaccatggtgttgttcttgatgacattgcttctgctaaatttaataaaaacttcagaaaaacctccattttgatcatcaggatttcatctgagtgtggagtccctggaatggaattcagtaacatttggagtgtgtattcaagtttctaaattgagattcgattactgtttggctgacatgacttttctggaagacatgatacacctactactcaattgttcttttcctttctctcgcccaacacgatcttgtaagatggatttcacccccaggccaatgcagctaattttgatagctgcattcatttatcaccagcatattgtgttctgagtgaatccactgtttgtcctgtcggatgcttgcttgattttttggcttcttatttctaagtagatagaaagcaataaaaatactatgaaatgaaagaacttgttcacaggttctgcgttacaacagtaacacatctttaatccgcctaattcttgttgttctgtaggttaaatgcaggtattttaactgtgtgaacgccaaactaaagtttacagtctttctttctgaattttgagtatcttctgttgtagaataataataaaaagactattaagagcaataaattatttttaagaaatcgagatttagtaaatcctattatgtgttcaaggaccacatgtgttctctattttgcctttaaatttttgtgaaccaattttaaatacattctcctttttgccctggattgttgacatgagtggaatacttggtttcttttcttacttatcaaaagacagcactacagatatcatattgaggattaatttatcccccctacccccagcctgacaaatattgttaccatgaagatagttttcctcaatggacttcaaattgcatctagaattagtggagcttttgtatcttctgcagacactgtgggtagcccatcaaaatgtaagctgtgctcctctcatttttatttttatttttttgggagagaatatttcaaatgaacacgtgcaccccatcatcactggaggcaaatttcagcatagatctgtaggatttttagaagaccgtgggccattgccttcatgccgtggtaagtaccacatctacaattttggtaaccgaactggtgctttagtaatgtggatttttttcttttttaaaagagatgtagcagaataattcttccagtgcaacaaaatcaattttttgctaaacgactccgagaacaacagttgggctgtcaacattcaaagcagcagagagggaactttgcactattggggtatgatgtttgggtcagttgataaaaggaaaccttttcatgcctttagatgtgagcttccagtaggtaatgattatgtgtcctttcttgatggctgtaatgagaacttcaatcactgtagtctaagacctgatctatagatgacctagaatagccatgtactataatgtgatgattctaaatttgtacctatgtgacagacattttcaataatgtgaactgctgatttgatggagctactttaagatttgtaggtgaaagtgtaatactgttggttgaactatgctgaagagggaaagtgagcgattagttgagcccttgccgggccttttttccacctgccaattctacatgtattgttgtggttttattcattgtatgaaaattcctgtgattttttttaaatgtgcagtacacatcagcctcactgagctaataaagggaaacgaatgtttcaaatcta

SEQ ID NO: 13 - protein, 1023 aa, Homo sapiens
MAGNDCGALLDEELSSFFLNYLADTQGGGSGEEQLYADFPELDLSQLDASDFDSATCFGELQWCPENSETEPNQYSPDDSELFQIDSENEALLAELTKTLDDIPEDDVGLAAFPALDGGDALSCTSASPAPSSAPPSPAPEKPSAPAPEVDELSLLQKLLLATSYPTSSSDTQKEGTAWRQAGLRSKSQRPCVKADSTQDKKAPMMQSQSRSCTELHKHLTSAQCCLQDRGLQPPCLQSPRLPAKEDKEPGEDCPSPQPAPASPQDSLALGRADPGAPVSQEDMQAMVQLIRYMHTYCLPQRKLPPQTPEPLPKACSNPSQQVRSRPWSRHHSKASWAEFSILRELLAQDVLCDVSKPYRLATPVYASLTPRSRPRPPKDSQASPGRPSSVEEVRIAASPKSTGPRPSLRPLRLEVKREVRRPARLQQQEEEDEEEEEEEEEEEKEEEEEWGRKRPGRGLPWTKLGRKLESSVCPVRRSRRLNPELGPWLTFADEPLVPSEPQGALPSLCLAPKAYDVERELGSPTDEDSGQDQQLLRGPQIPALESPCESGCGDMDEDPSCPQLPPRDSPRCLMLALSQSDPTFGKKSFEQTLTVELCGTAGLTPPTTPPYKPTEEDPFKPDIKHSLGKEIALSLPSPEGLSLKATPGAAHKLPKKHPERSELLSHLRHATAQPASQAGQKRPFSCSFGDHDYCQVLRPEGVLQRKVLRSWEPSGVHLEDWPQQGAPWAEAQAPGREEDRSCDAGAPPKDSTLLRDHEIRASLTKHFGLLETALEEEDLASCKSPEYDTVFEDSSSSSGESSFLPEEEEEEGEEEEEDDEEEDSGVSPTCSDHCPYQSPPSKANRQLCSRSRSSSGSSPCHSWSPATRRNFRCESRGPCSDRTPSIRHARKRREKAIGEGRVVYIQNLSSDMSSRELKRRFEVFGEIEECEVLTRNRRGEKYGFITYRCSEHAALSLTKGAALRKRNEPSFQLSYGGLRHFCWPRYTDYDSNSEEALPASGKSKYEAMDFDSLLKEAQQSLH

SEQ ID NO: 14 - DNA, 3277 bp, Homo sapiens
ctcggcgttgactccgccgcacgctgcagccgcggctggaagatggcggggaacgactgcggcgcgctgctggacgaagagctctcctccttcttcctcaactatctcgctgacacgcagggtggagggtccggggaggagcaactctatgctgactttccagaacttgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactgctgcagaagctcctcctggccacatcctacccaacatcaagctctgacacccagaaggaagggaccgcctggcgccaggcaggcctcagatctaaaagtcaacggccttgtgttaaggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccaggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccgtcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtgggtgtggggacatggatgaggaccccagctgcccgcagctccctcccagagactctcccaggtgcctcatgctggccttgtcacaaagcgacccaacttttggcaagaagagctttgagcagaccttgacagtggagctctgtggcacagcaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggtgccccacccaaggacagcacgctgctgagagaccatgagatccgtgctagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattgataacagccttaaccctcgaggaatacctcaatacctcagacaaggcccttccaatatgtttacgttttcaaagaaatcaagtatatgaggagagcgagcgagcgtgagagaacacccgtgagagagacttgaaactgctgtcctttaaaaaaaaaaaaaaaaa

(19) United States
(12) Patent Application Publication
(10) Pub. No.: US 2010/0310574 A1
(43) Pub. Date: Dec. 9, 2010

(54) METHODS AND COMPOSITIONS FOR THE TREATMENT AND DIAGNOSIS OF STATIN-INDUCED MYOPATHY

(75) Inventors: Vikas P. Sukhatme, Newton, MA (US); Stewart H. Lecker, Brookline, MA (US); Junichi Hanai, Boston, MA (US)

Correspondence Address: CLARK & ELBING LLP, 101 FEDERAL STREET, BOSTON, MA, 02110, US

(73) Assignee: Beth Israel Deaconess Medical Center, Boston, MA (US)

(21) Appl. No.: 12/663,423

(22) PCT Filed: Jun. 5, 2008
(86) PCT No.: PCT/US08/07047

§ 371 Date: Aug. 31, 2010

Related U.S. Application Data
(60) Provisional application No. 60/943,521, filed on Jun. 12, 2007.

(51) Int. Cl. A61K 39/395; A61K 38/02; A61K 31/675; A61K 31/7105; G01N 33/68; C12Q 1/68; G01N 33/566; G01N 33/53; C12N 5/07; A61P 21/00

(52) U.S. Cl. 424/158.1; 514/1.1; 514/81; 514/44 A; 435/7.1; 435/6; 436/501; 436/86; 436/94; 435/375

(57) ABSTRACT

The invention provides compositions and kits containing an atrogin-1 inhibitor compound useful for the treatment of a statin-mediated myopathy. Kits for the diagnosis of a statin-mediated myopathy are also provided. The invention further features methods for treating or preventing a statin-mediated myopathy in a subject via administration of a therapeutically effective amount of an atrogin-1 inhibitor compound. The invention further provides methods of diagnosing a subject as having a propensity to develop a statin-induced myopathy and methods of monitoring a statin-induced myopathy or a propensity to develop a statin-mediated myopathy in a subject. The invention also features methods for identifying a compound for the treatment of a statin-mediated myopathy and methods of identifying a statin compound as having the propensity to induce a statin-mediated myopathy.

### BACKGROUND OF THE INVENTION

The invention relates to methods and compositions for the treatment and diagnosis of statin-mediated myopathies.

Statins is the common name for the class of drugs termed 3-hydroxy-3-methylglutaryl coenzyme A (HMG CoA) reductase inhibitors. These drugs lower levels of low-density lipoprotein cholesterol and, as a treatment, have successfully reduced the risk of adverse cardiovascular events and coronary heart disease in dyslipidemic patient populations. Over 100 million patients worldwide are taking these drugs, and statin manufacturing and therapy is a multibillion-dollar industry. The success of statin therapy, however, has been tempered by drug-induced side effects including muscle toxicity. This condition has been termed statin-mediated myopathy and is thought a relatively infrequent but often debilitating complication of this otherwise successful therapeutic approach. The most serious of these complications is rhabdomyolysis, or muscle breakdown, which can result in kidney failure. More controversial is a milder version of this disease characterized by muscle pain (myalgias) or an inflammation of the muscles (myositis) with or without evidence of muscle damage as assessed by creatine kinase (CK) serum elevation. The incidence of statin-associated or induced myalgia or myositis is generally thought to be between 1-5% of the patient population that is on statin therapy. However, some investigators believe that the incidence of these myopathies is much higher, between 10 and 20% of patients on statin therapeutic regimens. Mechanisms mediating statin-mediated myopathy remain unclear, and currently there are no treatments, nor is there an established protocol for diagnosis of patients suffering from this disorder.

The muscle atrophy program is well established in several disease states including advanced cancer (tumor cachexia), sepsis, diabetes, and other systemic diseases. Muscle atrophy is also a debilitating side effect associated with several additional disorders including inactivity, denervation, and food deprivation and fasting. More recently, a variety of drugs have been associated with varying degrees of myotoxicity, including myopathy, and ultimately muscle atrophy in its most severe form. The atrophy process usually manifests as a rapid loss of muscle mass where rates of cellular protein synthesis are surpassed by rates of cellular protein degradation. The cellular protein degradation program mobilizes muscle protein as a source of catabolic amino acids for gluconeogenesis, or other stress-induced metabolic requirements. Despite diverging etiologies, myopathy and muscle atrophy in many diseases share common biochemical and transcriptional pathways, including ubiquitination and targeted proteosomal breakdown of muscle proteins.

Proteins destined for degradation by the ubiquitin (Ub)-proteasome pathway are first covalently linked to a chain of Ub molecules, which marks them for rapid breakdown to short peptides by the 26S proteasome. The key enzyme responsible for attaching Ub to protein substrates is a Ub-protein ligase (E3) that catalyzes the transfer of an activated form of Ub from a specific Ub-carrier protein (E2) to a lysine residue on the substrate protein. Individual E3s ubiquitinate specific classes of proteins; hence, the identity of the proteins degraded by the proteasome is largely determined by the complement of E3s active in individual cells. Atrogin is one example of an E3 ligase that has been identified in muscle cells and shown to be upregulated in atrophying muscle cells. What role atrogin-1 may have in muscle cells or in myopathies of any sort remains unknown.

There exists the need for therapeutics to prevent or treat statin-mediated myopathies. Furthermore, there is a need for techniques for diagnosing a statin mediated-myopathy and for monitoring patients undergoing treatment for a statin-mediated myopathy.

### SUMMARY OF THE INVENTION

The invention provides methods of treating or preventing a statin-induced myopathy in a subject by administering a therapeutically effective amount of an atrogin-1 inhibitor compound in an amount and for a time sufficient to treat or prevent a statin-mediated myopathy in a subject (e.g., human). In one aspect of the invented method, the atrogin-1 inhibitor compound reduces or inhibits the expression levels or biological activity (e.g., ubiquitin ligase activity, substrate binding activity, and nuclear translocation) of an atrogin-1 protein or an atrogin-1 nucleic acid. In some embodiments of the method, the statin may be administered at a high dosage or administered for extended release. In additional embodiments of the method, the subject has been treated with a statin, is still being treated with a statin, or will be treated with a statin.

Additional embodiments of the method of the invention include administration of an atrogin-1 inhibitor compound simultaneously or sequentially with a statin, administration of an atrogin-1 inhibitor compound following a statin, administration of an atrogin-1 inhibitor compound prior to administration of a statin, and administration of an atrogin-1 inhibitor compound following cessation or termination of statin administration.

The invention also provides compositions containing an atrogin-1 inhibitor compound that reduces or inhibits the expression or biological activity of atrogin-1, wherein the atrogin-1 inhibitor compound is formulated for the treatment or prevention of a statin-mediated myopathy. In a related aspect of the invented composition, the atrogin-1 inhibitor compound reduces or inhibits the expression levels or biological activity (e.g., ubiquitin ligase activity, substrate binding activity, and nuclear translocation) of an atrogin-1 polypeptide or an atrogin-1 nucleic acid. In an additional embodiment of the composition, the atrogin-1 inhibitor compound specifically binds atrogin-1 and reduces or inhibits the biological activity of atrogin-1.

The invention also provides kits containing a statin, an atrogin-1 inhibitor compound, and instructions for administration of the statin and the atrogin-1 inhibitor compound for the treatment or prevention of a statin-mediated myopathy.

In an additional aspect, the invention provides kits containing an atrogin-1 inhibitor compound and instructions for administration of the atrogin-1 inhibitor compound for the treatment of a statin-induced myopathy.

The invention further provides methods of diagnosing a subject as having or having a propensity to develop a statin-mediated myopathy, the method requiring measuring the level of an atrogin-1 polypeptide, atrogin-1 nucleic acid, or fragments thereof, in a sample from a subject relative to a reference sample or level (e.g., normal reference sample or level), wherein an alteration (e.g., increase) in the subject levels relative to the reference sample or level is diagnostic of a statin-mediated myopathy or a propensity to develop a statin-mediated myopathy in a subject. In different embodiments of the method, the atrogin-1 polypeptide is measured using an immunological assay, enzymatic assay, or colorimetric assay. In different embodiments of the method, the sample is a bodily fluid, tissue, or a cell (e.g., a myocyte) from a subject.

In addition, the invention provides methods of diagnosing a subject as having a propensity to develop a statin-mediated myopathy requiring measuring the level of an antibody, or a fragment thereof, that specifically binds atrogin-1 in a blood or serum sample from a subject relative to a reference level (e.g., normal reference level), wherein an alteration (e.g., increase) in the subject levels compared to the reference level is diagnostic of a statin-mediated myopathy or a propensity to develop a statin-mediated myopathy in the subject. In different embodiments of the method, the antibody, or fragment thereof, that specifically binds atrogin-1 polypeptide, or fragment thereof, is measured using an immunological assay and an atrogin-1 polypeptide, or fragment thereof, as a substrate.

The invention further provides a method of monitoring a statin-mediated myopathy or a propensity to develop a statin-mediated myopathy in a subject, wherein the method requires measuring the level of an atrogin-1 polypeptide, nucleic acid, atrogin-1 specific antibody, or fragments thereof in a sample from a subject (e.g., bodily fluid, a tissue, or a cell), and comparing the level to a reference sample or level, wherein an alteration in the level is an indicator of a change in the propensity to develop a statin-mediated myopathy, or a change in a statin-mediated myopathy of the subject. In different embodiments, the method is used to monitor a subject during treatment of a statin-mediated myopathy or monitor a subject at risk for a statin-mediated myopathy. In an additional embodiment of the method, the reference is a positive reference and a decrease in level is indicative of improvement.

In an additional aspect, the invention provides a kit for the diagnosis of a statin-mediated myopathy containing an atrogin-1 binding protein (e.g., antibody or an antigen-binding fragment thereof) and instructions for the use of the atrogin-1 binding protein for the diagnosis of a statin-mediated myopathy in a subject.

The invention further provides a kit for the diagnosis of a statin-mediated myopathy containing a nucleic acid complementary to at least a portion of an atrogin-1 nucleic acid molecule, wherein the nucleic acid molecule hybridizes at high stringency to atrogin-1, and instructions for the use of the nucleic acid for the diagnosis of statin-mediated myopathy in a subject.

In an additional aspect, the invention provides a kit for the diagnosis of a statin-mediated myopathy containing a polypeptide that specifically binds an atrogin-1 antibody or fragment thereof, and instructions for the use of the polypeptide for the diagnosis of statin-mediated myopathy in a subject.

In any of the above kits for the diagnosis of a statin-mediated myopathy, the kit may be used to monitor a statin-mediated myopathy in a subject that already has or is at risk for developing statin-mediated myopathy, or may be used to monitor the treatment of a subject for statin-mediated myopathy. Any of the above kits may also be used to determine the therapeutic dosage of a statin.

The invention also provides a method of identifying a compound for the treatment of a statin-mediated myopathy requiring contacting a cell (e.g., a myocyte) with a statin compound and further contacting the cell with a candidate compound, and comparing the level of expression of an atrogin-1 polypeptide in the cell contacted by the statin compound and the candidate compound with the level of expression in a control cell contacted by the statin compound, wherein a decrease in expression of the atrogin-1 polypeptide in the cell as compared to the control cell identifies the candidate compound as a candidate compound for the treatment of a statin-mediated myopathy.

In an additional aspect, the invention provides a method of identifying a compound for the treatment of a statin-mediated myopathy requiring contacting a cell (e.g., a myocyte) with a statin compound and further contacting the cell with a candidate compound, and comparing the level of expression of an atrogin-1 nucleic acid in the cell contacted by the statin compound and the candidate compound with the level of expression in a control cell contacted by the statin compound, wherein an decrease in expression of the atrogin-1 nucleic acid in the cell as compared to the control cell identifies the candidate compound as a compound for the treatment of a statin-mediated myopathy.

The invention also provides a method of identifying a compound for the treatment of a statin-mediated myopathy requiring contacting a cell (e.g., a myocyte) with a statin compound and further contacting the cell with a candidate compound, and comparing the biological activity of an atrogin-1 polypeptide in the cell contacted by the statin compound and the candidate compound with the biological activity in a control cell contacted by the statin compound, wherein a decrease in the biological activity of the atrogin-1 polypeptide in the cell as compared to the control cell identifies the candidate compound as a compound for the treatment of a statin-mediated myopathy. In different embodiments of the method, the atrogin-1 biological activity is ubiquitin ligase activity, substrate binding activity, or nuclear translocation.

In an additional aspect, the invention provides a method of identifying a statin compound as having the propensity to induce a statin-mediated myopathy requiring contacting a cell (e.g., a myocyte) with a statin compound, and comparing the level of expression of an atrogin-1 polypeptide in the cell contacted by the statin compound with the level of expression in a control cell not contacted by the statin compound, wherein an increase in expression of the atrogin-1 polypeptide in the cell as compared to the control cell identifies the statin compound as having the propensity to induce a statin-mediated myopathy.

The invention also provides a method of identifying a statin compound as having the propensity to induce a statin-mediated myopathy requiring contacting a cell (e.g., a myocyte) with a statin compound, and comparing the level of expression of an atrogin-1 nucleic acid in the cell contacted by the statin compound with the level of expression in a control cell not contacted by the statin compound, wherein a decrease in expression of the atrogin-1 nucleic acid in the cell as compared to the control cell identifies the statin compound as having the propensity to induce a statin-mediated myopathy.

In addition, the invention provides a method of identifying a statin compound as having the propensity to induce a statin-mediated myopathy requiring contacting a cell (e.g., a myocyte) with a statin compound, and comparing the biological activity of an atrogin-1 polypeptide (e.g., ubiquitin ligase activity, substrate binding activity, or nuclear translocation) in a control cell not contacted by the statin compound, wherein an increase in the biological activity of the atrogin-1 polypeptide in the cell compared to the control cell identifies the statin compound as having the propensity to induce a statin-mediated myopathy.

In an additional aspect, the invention provides a method of treating a biological sample from a subject having a statin-induced myopathy or the propensity to develop a statin-induced myopathy requiring removing a biological sample from a subject having a statin-induced myopathy or the propsensity to develop a statin-induced myopathy and treating the biological sample with a therapeutically effective amount of an atrogin-1 inhibitor compound ex vivo. Some embodiments of the method further require reintroducing the treated biological sample back in the subject having a statin-induced myopathy or the propensity to develop a statin-induced myopathy.

In different embodiments of all the above aspects of the invention, the atrogin-1 inhibitor compound specifically binds atrogin-1 (e.g., specifically binds the ubiquitin ligase domain, the substrate-binding domain, or the N- or C-terminal nuclear localization sequence). In additional embodiments of all the above aspects of the invention, the atrogin-1 inhibitor compound is an antibody or antigen-binding fragment thereof (e.g., monoclonal antibody, polyclonal antibody (e.g., anti-atrogin-1 IgG), a single-chain antibody, a chimeric antibody, a humanized antibody, a fully humanized antibody, a human antibody, or a bispecific antibody) that specifically binds atrogin-1.

In different examples of all the above embodiments of the invention, the atrogin-1 inhibitor compound reduces or inhibits the expression levels of an atrogin-1 nucleic acid molecule. In different embodiments of all the above aspects of the invention, the atrogin-1 inhibitor compound is: an aptamer that specifically binds atrogin-1; an antisense nucleobase oligomer (e.g., 8 to 30 nucleotides in length) that contains a nucleic acid substantially identical to at least a portion of an atrogin-1 nucleic acid molecule, or a complementary sequence thereof; a morpholino oligomer that is complementary to at least a portion of an atrogin-1 nucleic acid molecule (e.g., a sequence substantially identical to SEQ ID NO: 8 or SEQ ID NO: 9); or a small RNA (e.g., 15 to 32 nucleotides in length) having at least one strand that includes a nucleic acid sequence substantially identical to at least a portion of an atrogin-1 nucleic acid molecule (e.g., a sequence substantially identical to a translational start site or a splicing site of an atrogin-1 nucleic acid molecule), or a complementary sequence thereof.

In different embodiments of all the above aspects of the invention, the statin is any pharmaceutical compound that inhibits HMG-CoA reductase (e.g., cerivastatin, simvastatin, atrovastin, fluvastatin, pravastatin, rosuvastatin, pitavastatin, lovastatin, compactin, mevinolin, mevastatin, velostatin, synvinolin, rivastatin, or verivastatin.

By “alteration” is meant a change (i.e., increase or decrease). The alteration can indicate a change in the expression levels of an atrogin-1 nucleic acid or polypeptide as detected by standard art known methods such as those described below. As used herein, an alteration includes a 10% change in expression levels, preferably a 25% change, more preferably a 40%, 50%, 60%, 70%, 80%, 90%, 95%, 96%, 97%, 98%, 99%, or greater change in expression levels. The alteration can also indicate a change (i.e., increase or decrease) in the biological activity of an atrogin-1 nucleic acid or polypeptide. As used herein, an alteration includes a 10% change in biological activity, preferably a 25% change, more preferably a 40%, 50%, 60%, 70%, 80%, 90%, 95%, 96%, 97%, 98%, 99%, or greater change in biological activity. Examples of biological activity for atrogin-1 polypeptides are described below.

By “antisense nucleobase oligomer” is meant a nucleobase oligomer, regardless of length, that is complementary to at least a portion of the coding strand or mRNA of an atrogin-1 gene. By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified and unmodified, as well as oligonucleotide mimetics such as protein Nucleic Acids, locked nucleic acids, and arabinonucleic acids. Numerous nucleobases and linkage groups may be employed in the nucleobase oligomers of the invention, including those described in U.S. Patent Publication Nos. 20030114412 (see for example paragraphs 27-45 of the publication) and 20030114407 (see for example paragraphs 35-52 of the publication), incorporated herein by reference. The nucleobase oligomer can also be targeted to the translational start and stop sites within an mRNA expressing atrogin-1 or to a splicing sequence within an atrogin-1 mRNA. Preferably the antisense nucleobase oligomer comprises from about 8 to 30 nucleotides. The antisense nucleobase oligomer can also contain at least 40, 60, 85, 120, or more consecutive nucleotides that are complementary to atrogin-1 mRNA or DNA, and may be as long as the full-length mRNA or gene. Examples of nucleobase oligomers are morpholino oligonucleotides, which have bases similar to natural nucleic acids, but are bound to morpholine rings instead of deoxyribose rings and are linked through phosphorodiamidate groups instead of phosphates. Morpholino oligonucleotides can be designed to any sequence of a target mRNA sequence (e.g., translation start site, an intron sequence, an exon sequence, or a splicing site). Morpholino oligonucleotides can be designed to target the mRNA sequences of any of the atrogenes (e.g., human atrogin-1) discussed herein.

By “atrogene” or “atrogenes” is meant a member of the family of proteins or a nucleic acid encoding the proteins, involved in the common biochemical and transcriptional atrophy program. This includes, but is not limited to, atrogin-1, a F-box protein regulated by the Forkhead box 0 (Foxo) family of transcription factors, which are also may be acknowledged as atrogenes. Foxo-1 induction has been demonstrated in most, if not all forms of atrophy. Foxo-3 (FoxO3) has been shown to regulate the expression of atrogin-1, and as such, is also included in the family of atrogenes. The Foxo family of proteins are tightly regulated by PI3K/AKT dependent phosphorylation, which may be considered upstream accessory components of the atrophy program. An additional member of the atrogene family may be MuRF-1, an additional ubiquitin E3 ligase.

By “atrogin-1” is meant a polypeptide, or a nucleic acid sequence that encodes it, or fragments or derivatives thereof, that is substantially identical to atrogin-1 nucleic acid or polypeptide sequences set forth in Genbank Accession Nos: BCO24030 and AAH24030 (human atrogin-1 mRNA and protein, respectively), BCO27211 and AAH27211 (mouse atrogin-1 mRNA and protein, respectively), or SEQ ID NO: 10 (zebrafish atrogin-1 protein). Atrogin-1 can also include fragments, derivatives, homologs, sequence variants, splice variants, or analogs of atrogin-1 that retain at least 25%, 30%, 40%, 50%, 60%, 70%, 80%, 90%, 95%, 96%, 97%, 98%, 99%, or more atrogin-1 biological activity.

An atrogin-1 polypeptide or nucleic acid molecule may be isolated from a variety of sources, such as from mammalian tissue or cells (e.g., myocytes) or from another source, or prepared by recombinant or synthetic methods. The term “atrogin-1” also encompasses modifications to the polypeptide, fragments, derivatives, analogs, and variants of the atrogin-1 polypeptide.

“Atrogin-1 biological activity” can include one or more of the following exemplary activities: substrate binding activity (e.g., calcineurin A), ubiquitin ligase activity, inhibition of calcineurin A activity, and nuclear translocation. Assays for atrogin-1 biological activity include assays for ubiquitination, substrate binding assays, calcineurin A activity assays, nuclear translocation, and other assays.

By “atrogin-1 inhibitor compound” is meant any small molecule chemical compound (peptidyl or non-peptidyl), antibody, nucleic acid molecule, polypeptide, or fragments thereof that reduces or inhibits the expression levels or biological activity of atrogin-1 by at least 10%, 20%, 30%, 40%, 50%, 60%, 70%, 80%, 90%, or more. Non-limiting examples of atrogin-1 inhibitor compounds include fragments of atrogin-1 (e.g., dominant negative fragments or fragments that lack or have decreased ubiquitin ligase activity, substrate binding activity, calcineurin A inhibition, or nuclear translocation); peptidyl or non-peptidyl compounds that specifically bind atrogin-1 (e.g., antibodies or antigen-binding fragments thereof), for example, at the ubiquitin ligase domain, substrate binding domain, or the N- or C-terminal nuclear localization sequences of atrogin-1 (amino acids 62-66 and amino acids 267-288 of atrogin-1); antisense nucleobase oligomers; morpholino oligonucleotides (e.g., SEQ ID NO: 8 and SEQ ID NO: 9, or those molecules which target the translation start sequence or splicing sequence of an atrogin-1 mRNA); small RNAs; small molecule inhibitors; compounds that decrease the half-life of atrogin-1 mRNA or protein; compounds that decrease transcription or translation of atrogin-1; compounds that reduce or inhibit the expression levels of atrogin-1 polypeptides or decrease the biological activity of atrogin-1 polypeptides (e.g., PGC-1α or PGC-1β protein); compounds that increase the expression or biological activity of a second atrogin-1 inhibitor (e.g., a molecule which increases the transcription or translation of PGC-1α or PGC-1β protein such as metformin); compounds that block atrogin-1-mediated downstream activities (e.g., ubiquitination; binding to SCF family members Skp-1 Cul-1, Roc-1; inhibition of calcineurin A activity; and nuclear translocation), and any compound that alters activities upstream of atrogin-1 (e.g., Foxo phosphorylation and PI3K/Akt phosphorylation). Atrogin-1 inhibitor compounds can be identified by testing the compound in any of the assays described herein or known in the art for atrogin-1 expression level or biological activity, and identifying a compound that shows at least a 10%, 15%, 20%, 25%, 30%, 35%, 40%, 45%, 50%, 55%, 60%, 65%, 70%, 75%, 80%, 85%, 90%, 95% or more decrease in atrogin-1 expression level or activity as compared to a control where the compound has not been added.

By “atrogin-1 substrate” is meant any protein or molecule that binds to atrogin-1 (e.g., calcineurin A).

By “compound” is meant any small molecule chemical compound, antibody, nucleic acid molecule, or polypeptide, or fragments thereof.

By “decrease” is meant to reduce, preferably by at least 20%, more preferably by at least 30%, and most preferably by at least 40%, 50%, 60%, 70%, 75%, 80%, 85%, 90%, 95%, or more. Decrease can refer, for example, to the symptoms of the disorder being treated or to the levels or biological activity of atrogin-1.

By “effective amount” is meant an amount sufficient to prevent or reduce a statin-mediated myopathy or any symptom associated with a statin-mediated myopathy. It will be appreciated that there will be many ways known in the art to determine the therapeutic amount for a given application. For example, the pharmacological methods for dosage determination may be used in the therapeutic context.

By “efficacy” is meant the effectiveness of a particular treatment regime. For example, efficacy in treating or preventing a statin-mediated myopathy can be measured by a reduction in any one or more of the following clinical or subclinical symptoms: atrogin-1 expression or biological activity, creatine kinase (CK) enzyme levels, overt necrosis of myocytes as evidenced by muscle biopsy, myalgia, musculoskeletal pain, muscle pain, musculoskeletal/connective tissue symptoms, or reduction of microglobinuria or transaminase levels with respect to rhabdomyolysis and hepatotoxicity.

By “expression” is meant the detection of a nucleic acid molecule or polypeptide by standard art known methods. For example, polypeptide expression is often detected by Western blotting, DNA expression is often detected by Southern blotting or polymerase chain reaction (PCR), and RNA expression is often detected by Northern blotting, PCR, or RNAse protection assays.

By “extended release” is meant formulation of a statin compound such that the release of the active agent (i.e., statin compound), when in combination with another non-active substance (e.g., binder, filler, protein, or polymer), into a physiological buffer (e.g., water or phosphate buffered saline) is decreased relative to the agent's rate of diffusion through a physiological buffer when the agent is not formulated with a non-active substance. Extended release formulations may decrease the rate of release of a statin compound by at least 10%, 20%, 30%, 40%, 50%, 60%, 70%, 80%, 90%, or 100% compared to the rate of release of a statin formulation which does not contain a non-active substance (e.g., binder, filler, protein, or polymer).

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain 10, 20, 30, 40, 50, 60, 70, 80, 90, or 100, 200, 300, 400, 500, 600, or more up to 627 nucleotides or 10, 20, 30, 40, 50, 60, 70, 80, 90, 100, 110, 120, 130, 140, 150, 160, 170, 180, 190, 200, or more up to 209 amino acids. Preferred fragments of atrogin-1 useful as atrogin-1 inhibitor compounds will reduce or inhibit atrogin-1 expression or biological activity (e.g., ubiquitin ligase activity, substrate binding activity, calcineurin A inhibition, or nuclear translocation).

By “heterologous” is meant any two or more nucleic acid or polypeptide sequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

By “high dosage” of a statin compound is meant administration of a statin compound to a subject at a total dose of greater than 10 mg/day, 20 mg/day, 30 mg/day, 40 mg/day, 50 mg/day, 60 mg/day, 70 mg/day, or 80 mg/day. The total dose administered to a patient in a single day may occur via separate dose units (examples include, but are not limited to, two pills, three pills, and a cream and a pill). A patient may be treated with a high dosage of a statin compound for any duration of time (e.g., more than one day, more than one week, more than one month, and more than one year). Any of the statins described herein are contemplated for administration at high dosage.

By “homologous” is meant any gene or polypeptide sequence that bears at least 30% identity, more preferably at least 40%, 50%, 60%, 70%, 80%, and most preferably at least 90%, 95%, 96%, 97%, 98%, 99%, or more identity to a known gene or polypeptide sequence over the length of the comparison sequence. A “homologous” polypeptide can also have at least one biological activity of the comparison polypeptide. For polypeptides, the length of comparison sequences will generally be at least 16 amino acids, preferably at least 20 amino acids, more preferably at least 30, 40, 50, 60, 70, 80, 90, 100, 110, 120, 130, 140, 150, 160, 170, 180, 190, 200, 209, or more amino acids. For nucleic acids, the length of comparison sequences will generally be at least 50 nucleotides, preferably at least contain 10, 20, 30, 40, 50, 60, 70, 80, 90, or at least 100, 200, 300, 400, 500, 600, 627, or more nucleotides. “Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein or polypeptide.

By “increase” is meant to augment, preferably by at least 20%, more preferably by at least 50%, and most preferably by at least 70%, 75%, 80%, 85%, 90%, 95%, or more. Increase can refer, for example, to the levels or biological activity of atrogin-1.

By “metric” is meant a measure. A metric may be used, for example, to compare the levels of a polypeptide or nucleic acid molecule of interest. Exemplary metrics include, but are not limited to, mathematical formulas or algorithms, such as ratios. The metric to be used is that which best discriminates between levels of atrogin-1 polypeptide in a subject having a statin-mediated myopathy and a normal reference subject. Depending on the metric that is used, the diagnostic indicator of a statin-mediated myopathy may be significantly above or below a reference value (e.g., from a control subject not having myopathy or undergoing statin therapy).

By “myopathy” is meant a disease of the muscle or muscle tissue. Non-limiting examples include congenital myopathies, muscular dystrophies, inflammatory myopathies, mitochondrial myopathies (e.g., Kearns-Sayre syndrome, MELAS, and MERRF), Pompe's disease, Andersen's disease, Cori's disease, myoglobinureas (e.g., McArdle, Tarui, and DiMauro diseases), myositis ossificans, dermatomyositis, familial periodic paralysis, polymyositis, inclusion body myositis, neuromyotonia, stiff-man syndrome, and tetany.

By “PGC-1α” is meant a polypeptide or nucleic acid substantially identical (e.g., at least 70%, 80%, 90%, 95%, or at least 99% identical) to peroxisome proliferator-activated receptor gamma coactivator-1-alpha (Genebank Accession Nos. NP_{—}037393 and NM_{—}013261, respectively). One activity of a PGC-1α polypeptide is to decrease the expression levels or biological activity of atrogin-1 polypeptides.

By “PGC-1β” is meant a polypeptide or nucleic acid substantially identical (e.g., at least 70%, 80%, 90%, 95%, or at least 99% identical) to peroxisome proliferator-activated receptor gamma coactivator-1-beta (Genebank Accession Nos. NP_{—}573570 and NM_{—}133263, respectively). One activity of a PGC-1β polypeptide is to decrease the expression levels or biological activity of atrogin-1 polypeptides.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, LippincOtt, Williams & Wilkins, Philadelphia, Pa.

By “positive reference” is meant a biological sample, for example, a biological fluid (e.g., urine, blood, serum, plasma, or cerebrospinal fluid), tissue (e.g., muscle tissue), or cell (e.g., myocyte), collected from a subject who has a myopathy (e.g., a statin-induced myopathy) or a propensity to develop a statin-induced myopathy (e.g., a statin-induced myopathy). A positive reference may also be a biological sample derived from a patient with a statin-induced myopathy prior to or during treatment. In addition, a positive reference may be derived from a subject that is known to have a statin-mediated myopathy, that is matched to the sample subject by at least one of the following criteria: age, weight, BML disease stage, overall health, prior diagnosis of a statin-mediated myopathy, and a family history of statin-mediated myopathy. A positive reference as used herein may also be purified atrogin-1 polypeptide (e.g., recombinant or non-recombinant atrogin-1 polypeptide), purified atrogin-1 nucleic acid, purified anti-atrogin-1 antibody, or any biological sample (e.g., a biological fluid, tissue, or cell) that contains atrogin-1 polypeptide, atrogin-1 nucleic acid, or an anti-atrogin-1 antibody. A standard curve of levels of purified atrogin-1 protein, purified atrogin-1 nucleic acid, or an anti-atrogin-1 antibody within a positive reference range can also be used as a reference.

By “preventing” is meant prophylactic treatment of a subject who is not yet ill, but who is susceptible to, or otherwise at risk of, developing a particular disease. Preferably, a subject is determined to be at risk of developing a statin-induced myopathy. “Preventing” can refer to the preclusion of a statin-mediated myopathy in a patient receiving a statin compound for the treatment or prevention of elevated blood cholesterol levels (e.g., LDL), hyperlipidemia, heart disease, stroke, heart attack, atherosclerosis, intermittent claudication, hypertension, coronary artery disease, type 1 (insulin dependent diabetes or IDDM) and type 2 (non-insulin-dependent diabetes or NIDDM) diabetes, and other related disease states. For example, the preventive measures are used to prevent a statin-mediated myopathy in a patient undergoing a statin therapy with a statin that has been identified as, or associated with, developing myopathy in the patient population. “Preventing” can also refer to the preclusion of the worsening of the symptoms of a statin-induced myopathy. For example, a compound of the invention can be used to prevent a mild statin myopathy (e.g., characterized by muscle pain (myalgias) or an inflammation of the muscles (myositis)) from developing into rhabdomyolysis (i.e. or e.g., muscle breakdown).

By “protein,” “polypeptide,” or “polypeptide fragment” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “reduce or inhibit” is meant the ability to cause an overall decrease preferably of 20% or greater, more preferably of 50% or greater, and most preferably of 65%, 70%, 75%, 80%, 85%, 90%, 95%, or greater. Reduce or inhibit can refer to the symptoms of the statin-mediated myopathy being treated, the levels of atrogin-1 polypeptide or nucleic acid, the levels of creatine kinase measured in a patient sample, or the amount of muscle or tissue loss in advanced or more serious statin-mediated myopathies (rhabdomyolysis). For diagnostic or monitoring applications, reduce or inhibit can refer to the level of protein or nucleic acid, detected by the aforementioned assays (see “expression”).

By “reference sample” is meant any sample, standard, or level that is used for comparison purposes. A “normal reference sample” can be, for example, a prior sample taken from the same subject; a sample from a subject not having a statin-mediated myopathy; a subject not treated with a statin; a subject that is diagnosed with a propensity to develop a statin-mediated myopathy but does not yet show symptoms of the disorder; a subject that has been treated for a statin-mediated myopathy; or a sample of a purified reference atrogin-1 polypeptide or nucleic acid molecule at a known normal concentration.

By “reference standard or level” is meant a value or number derived from a reference sample. A normal reference standard or level can be a value or number derived from a normal subject who does not have a statin-mediated myopathy. In preferred embodiments, the reference sample, standard, or level is matched to the sample subject by at least one of the following criteria: age, weight, body mass index (BMI), disease stage, and overall health. A “positive reference” sample, standard, or value is a sample, value, or number derived from a subject that is known to have a statin-mediated myopathy, that is matched to the sample subject by at least one of the following criteria: age, weight, BMI, disease stage, overall health, prior diagnosis of a statin-mediated myopathy, and a family history of statin-mediated myopathy. A standard curve of levels of purified protein within the normal or positive reference range can also be used as a reference.

By “sample” is meant a bodily fluid (e.g., urine, blood, serum, plasma, or cerebrospinal fluid), tissue (e.g., muscle tissue), or cell (e.g., myocyte) in which the atrogin-1 polypeptide or nucleic acid molecule is normally detectable.

By “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds atrogin-1 does not bind other ubiquitin ligase or atrogene family members.

By “subject” is meant a mammal, including, but not limited to, a human or non-human mammal, such as a bovine, equine, canine, ovine, or feline.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., a atrogin-1 sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman, J. Mol. Biol. 147:195-7, 1981); “BestFit” (Smith and Waterman, Advances in Applied Mathematics, 482-489, 1981) as incorporated into GeneMatcher Plus™, Schwarz and Dayhof, “Atlas of Protein Sequence and Structure,” Dayhof, M. O., Ed pp 353-358, 1979; BLAST program (Basic Local Alignment Search Tool; (Altschul, S. F., W. Gish, et al., J. Mol. Biol. 215: 403-410, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10 amino acids, preferably 20, 30, 40, 50, 60, 70, 80, 90, 100, 110, 120, 130, 140, 150, 160, 170, 180, 190, 200, 209 amino acids or more. For nucleic acids, the length of comparison sequences will generally be at least 10, 20, 30, 40, 50, 60, 70, 80, 90, or 100, 200, 300, 400, 500, 600, 627, or more nucleotides. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “treating” is meant administering a compound or a pharmaceutical composition for prophylactic and/or therapeutic purposes or administering treatment to a subject already suffering from a disease to improve the subject's condition or to a subject who is at risk of developing a disease. As it pertains to statin-mediated myopathies, treating can include improving or ameliorating the symptoms of a statin-mediated myopathy and prophylactic treatment can include preventing the progression of a mild myopathy (e.g., myalgia and myositis) to a more serious form such as rhabdomyolysis. Prophylactic treatment can be monitored, for e.g., by measuring the CK levels in a subject undergoing prophylactic treatment and ensuring that the CK levels do not become significantly elevated or, desirably, to cause a detectable decrease in the CK levels. Treating may also mean to prevent the onset of a myopathy or the symptoms of a myopathy in a patient receiving a statin or a patient identified as at risk for developing a statin-induced myopathy (e.g., using the methods described herein).

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

By “statin-mediated myopathy” is meant the presence of clinical or subclinical symptoms of myopathy in a patient undergoing statin therapy. This includes muscle pain (myalgia) or an inflammation of the muscles (myositis) with or without evidence of muscle damage as assessed by CK elevations in the blood, serum, or plasma. This may accompany histological confirmation of necrosis by muscle biopsy. The recent American College of Cardiology/American Heart Association clinical advisory on the use and safety of statins defined four syndromes: statin myopathy (any muscle complaints related to these drugs); myalgia (muscle complaints without serum CK elevations); myositis (muscle symptoms with CK elevations); and rhabdomyolysis (markedly elevated CK levels, usually >10 times the upper limit of normal (ULN), with an elevated creatinine level consistent with pigment-induced nephropathy).

By a “statin compound” or “statin” is meant any a pharmaceutical compound that inhibits HMG-CoA reductase. Generally, statin compounds are understood to be those active agents which may be used to lower the lipid levels, including cholesterol, in the blood of a subject. The class of HMG-CoA reductase inhibitors includes both naturally occurring and synthetic molecules having differing structural features. Exemplary HMG-CoA reductase inhibitors include: atorvastatin, cerivastatin, fluvastatin, lovastatin, pitavastatin (formerly itavastatin), pravastatin, rosuvastatin (formerly visastatin), simvastatin, compactin, mevinolin, mevastatin, velostatin, cerivastatin, synvinolin, or rivastatin (sodium 7-(4-fluorophenyl) 2,6-diisopropyl-5-methoxymethylpyridin-3-yl)3,5-dihydroxy-6-heptanoate) or, in each case, a pharmaceutically acceptable salt thereof. This list is not restrictive and new molecules belonging to this large family are regularly discovered. A statin may be hydrophilic, like pravastatin, or lipophilic, like atorvastatin. Lipophilic statins are believed to better penetrate the tissues. A molecule which is “chemically related or structurally equivalent” to a statin includes molecules whose structure differs from that of any member of the statin family by 2 or fewer substitutions or by modification of chemical bonds. A molecule which is “functionally equivalent” to a statin includes molecules capable of measurable HMG-CoA reductase inhibition. Thus, at least all the molecules capable of competitively inhibiting the enzyme HMG-CoA reductase and called statins possess the required property.

Other features and advantages of the invention will be apparent from the following Detailed Description, the drawings, and the claims.

### BRIEF DESCRIPTION OF THE DRAWINGS

FIG. 1 is the amino acid sequence of human atrogin-1 (Genbank Accession No. AAH24030; SEQ ID NO: 1).

FIG. 2 is the nucleic acid sequence of human atrogin-1 mRNA (Genbank Accession No. BC024030; SEQ ID NO: 2).

FIG. 3 is the amino acid sequence of mouse atrogin-1 (Genbank Accession No. AAH27211; SEQ ID NO: 3).

FIG. 4 is the nucleic acid sequence of mouse atrogin-1 mRNA (Genbank Accession No. BC02721 1; SEQ ID NO: 4).

FIG. 5A is a graph showing the fold-induction of atrogin-1 mRNA expression in control, non-statin myopathy, and statin-treated human muscle samples quantitated by real-time PCR. The asterisk represents p<0.05 in statin-treated muscle vs. control muscle or non-statin myopathy muscle.

FIG. 5B is a graph showing the fold-induction of atrogin-1 mRNA expression in control, non-statin myopathy, and statin-treated human muscle samples quantitated by real-time PCR. The data for males (filled bars) and females (open bars) are shown.

FIG. 6A is a picture and histogram showing C2C12 myotube morphology and mean diameter following treatment with lovastatin at various concentrations (0-10 μM).

FIG. 6B is a picture and histogram showing C2C12 myotube morphology and mean diameter following treatment with lovastin for various periods of time (0-5 days).

FIG. 7A is a graph showing the relative atrogin-1 mRNA expression in C2C12 cells following treatment with 0, 1.0, 2.5, 5.0, and 10 μM lovastatin for 6, 20, and 36 hours, respectively.

FIG. 7B is an immunoblot and histogram showing the induction of atrogin-1 protein expression in C2C12 cells following treatment with 0, 1.0, 2.5, 5.0, and 10 μM lovastatin for 48 hours. Dexamethasone (5 μM) was used as a positive control.

FIG. 7C is a time course (1-5 days) showing atrogin-1 protein expression following 0-2.5 μM lovastatin treatment.

FIG. 7D is a graph showing the percent increase in protein degradation in cultures treated with lovastatin (1, 2.5, 5, or 10 μM) or cultures treated with 10 μM dexamethasone compared to untreated cultures.

FIG. 8A is an immunoblot showing atrogin-1 protein expression in myoblasts from atrogin-1 knockout mice (−/−) and wildtype mice (+/+) following treatment with dexamethasone (5 μM; dex) or infection with constitutively active FoxO3- or GFP-expressing adenovirus (ad-FoxO3 and ad-GFP, respectively).

FIG. 8B is a picture and a graph showing myotubes and the mean myotube diameter for atrogin-1 null (−/−) and wildtype (+/+) myotubes following treatment with 0, 0.01, 0.05, 0.25, 1.0, or 2.5 μM novastatin for 48 hours.

FIG. 9 is a comparison of the mouse (SEQ ID NO: 3) and zebrafish (SEQ ID NO: 10) atrogin-1 amino acid sequence.

FIG. 10A is a picture of the morphology of the myofiber structure in control zebrafish embryos and embryos treated with 0.025 to 5.0 μM lovastatin for 12 hours.

FIG. 10B is a histogram depicting the percentage of zebrafish embryos having class 1, class 2, and class 3 changes following exposure to 0, 0.025, 0.05, 0.5, 1.0, or 5 μM lovastatin. Class 1 changes include bowing, gap formation, and blocked/disrupted fibers. Class 2 changes include irregular fibers and diffuse appearance. Class 3 changes are typified by irregular somite boundries. The numbers of embryos quantitated for each treatment: 151, 178, 163, 185, 189, 180 for lovastatin concentrations of 0, 0.025, 0.05, 0.5, 1.0, and 5 μM respectively.

FIG. 11A is a photomicrograph of the myosin heavy chain staining of control, z-HMG CoA reductase knockdown, and z-HMG CoA reductase and atrogin-1 knockdown zebrafish myofibers.

FIG. 11B is a graph showing the percentage of damaged embryos with class 1, class 2, or class 3 morphological phenotypes following treatment with morpholinos MO 1 and MO2 (which target knockdown of HMG CoA reductase) in the presence or absence of atrogin-1 knockdown. The number of embryos quantitated for each condition: 231, 182, 197, 202 for wildtype atrogin-1 expression and 220, 204, 240, 215 for the atrogin-1 knockdown.

FIG. 12A is a blot depicting the level of z-HMG CoA reductase mRNA expression following treatment with a morpholino oligonucleotide (MO) designed against the common splice site or both splice variants of the zebrafish HMG CoA reductase gene.

FIG. 12B is a photomicrograph of the myosin heavy chain staining of control myofibers and myofibers from embryos with z-HMG CoA reductase knockdown following treatment with morpholino oligonucleotides targeting the splicing site of the z-HMG CoA reductase mRNA.

FIG. 13A is a blot depicting atrogin-1 mRNA expression in control zebrafish embryos and embryos treated with 0.5 μM lovastatin for 12 hours.

FIG. 13B is an immunoblot depicting the expression of atrogin-1 protein in control zebrafish embryos and embryos treated with 0.5 or 1.0 lovastatin for 12 hours.

FIG. 13C is an immunoblot depicting atrogin-1 protein expression in control zebrafish embryos; zebrafish embryos injected with a morpholino against atrogin-1; adult zebrafish muscle adult zebrafish kidney; and adult zebrafish liver.

FIG. 13D is a photomicrograph showing the myosin heavy chain staining of myofibers from representative control (WT) and antrogin-1 knockdown embryos following 0, 0.05, and 0.5 μM lovastatin treatment.

FIG. 13E is a graph showing the percentage of damaged embryos (control and atrogin-1 knockdown embryos) having class 1, class 2, or class 3 damage following 0-1.0 μM lovastatin treatment. The numbers of embryos quantitated are 235, 182, 197, 202 for the controls and 220, 204, 240, and 215 for the atrogin-1 knockdowns at lovastatin concentrations of 0, 0.05, 0.5, and 1.0 respectively.

FIG. 13F is the mean muscle fiber diameter measured by myosin heavy chain staining of myofibers from control and atrogin-1 knockdown zebrafish embryos following 0, 0.05, 0.5, and 1 μM lovastatin treatment. At least 500 fibers were measured for each lovastatin concentration. Results were graphed as a ratio of mean experimental fiber size+/−S.E.M. to control fiber size+/−S.E.M. Control fiber size is 7.60+/−0.19 μm.

FIG. 14A is an immunoblot showing the expression of phosphorylated Akt (p-Akt), Akt, phosphorylated FoxO3 (p-FoxO3), FoxO3, phophorylated p70S6K (p-p70S6K), p70S6K, and GADPH in C2C12 myotubes following 24-hour treatment with vehicle or lovastatin (1, 2.5, or 5 μM).

FIG. 14B is a graph showing the reporter gene expression from a FoxO-dependent promoter and FoxO site-mutated promoter in transfected embryos receiving no treatment (control) or treatment with 0.5 μM lovastatin for 48 hours. Transfected embryos coinjected with constitutively active FoxO3a (FoxO) were used as a positive control.

FIG. 15A is an immunoblot showing the expression of myc-PGC-1α in control-injected and myc-PGC-1α-injected zebrafish embryos 24 h and 48 h following injection.

FIG. 15B is a photomicrograph showing the cross-sectional anti-myc staining of representative control and myc-PGC-1α-injected zebrafish embryos.

FIG. 16A is a photomicrograph showing the myosin heavy chain staining of myofibers from representative zebrafish embryos following injection of 100 pg PGC-1α cDNA or vehicle in the presence or absence of 0.5 μM lovastatin treatment for 12 h (left box) or morpholino oligonucleotides against z-PGC-1α (right box).

FIG. 16B is a graph showing the percentage of damaged embryos having class 1, class 2, or class 3 myofiber damage following treatment of wildtype and PGC-1α cDNA-injected embryos with 0-1.0 μM lovastatin. The numbers of embryos quantitated are 137, 112, 139, 122 for controls (wildtype) and 120, 103, 108, 107 for PGC-1α-injected embryos at lovastatin concentrations of 0, 0.05, 0.5, and 1.0 μM, respectively.

FIG. 16C is an immunoblot showing the level of atrogin-1 protein expression in control and PGC-1α cDNA-injected (100 pg) zebrafish embryos left treated with vehicle or 1.0 μM lovastatin for 12 hours.

FIG. 16D is a graph of the mean muscle fiber diameter of zebrafish embryos injected with atrogin-1 morpholinos or PGC-1α cDNA in the presence or absence of 0.5 μM lovastatin treatment. At least 500 fibers were measured at each treatment. Results were graphed as the ratio of mean experimental fiber size+/−S.E.M. to mean control fiber size+/−S.E.M. Control fiber size was 7.58+/−0.10 μm.

FIG. 16E is a picture of the GFP fluorescence of C2C12 myotubes infected for 68 hours with control adenovirus or PGC-1α-adenovirus and treated with vehicle or 5 μM lovastatin.

FIG. 16F is an immunoblot showing the expression of atrogin-1, PGC-1α, cytochrome oxidase IV (cox IV), cytochrome c (cyto C), and dynein protein in control-infected or PGC-1α-infected C2C12 myotube cultures following treatment with 0-5 μM lovastatin for 48 hours.

FIG. 16G is a graph showing the fluorescence intensity of cells from zebrafish embryos treated with vehicle or lovastatin (0.5 or 1 μM) and stained with MitoTracker. Cellular fluorescence intensity was measured by fluorescence-activated cell sorting. Data presented as percent of mean fluorescence intensity in vehicle-treated embryos. Representative data from 3 independent experiments is shown.

FIG. 16H is a graph showing the fluorescence intensity of embryos non-injected or injected with PGC-1α cDNA, following treatment with vehicle or lovastatin (0.5 μM) for 24 hours and staining with MitoTracker. Cellular fluorescence intensity was measured by fluorescence-activated cell sorting and data presented as percent of mean fluorescence intensity in vehicle-treated embryos.

FIG. 16I is a raw fluorescence intensity tracing for the experimental data shown in FIG. 16G. Representative data from 3 independent experiments is shown.

FIG. 16J is a series of raw fluorescence intensity tracings for the experimental data shown in FIG. 16H.

FIG. 17 shows the result of a co-immunoprecipation experiment indicating the level of atrogin-1 protein in lysate from myc₆AT-1-transfected 293T cells (IgG: preimmune IgG; Anti-AT-1: anti-atrogin-1 IgG antibody).

FIG. 18 is a photomicrograph showing the subcellular localization of myc-atrogin-1 in Ad-myc₆-AT-1-transfected and control C2C12 myoblasts using an anti-myc antibody.

FIG. 19 is a photomicrograph showing the subcellular localization of myc-atrogin-1 following electroporation of Myc₆AT1 plasmid or control plasmid into the tibialis anterior muscle of fed or starved (48 hours) mice. Atroglin-1 protein was detected using an anti-myc antibody.

FIG. 20A is a diagram showing the location of the N- and C-terminal putative nuclear localization sequences in atrogin-1 (amino acids 62-66 and 267-288, respectively).

FIG. 20B is a graph showing the percentage of nuclear localization of wildtype myc-atrogin-1 (AT-1) or myc-atrogin-1 having a mutation in the N-terminal putative nuclear localization sequence (AT1-N), a mutation in the C-terminal putative nuclear localization sequence (AT1-C), or having a mutation in both the N- and C-terminal putative nuclear localization sequences (AT1-N+C).

FIG. 21 is the amino acid sequence of human PGC-1α polypeptide (Genbank Accession No. NP_{—}037393; SEQ ID NO: 11).

FIG. 22 is the nucleic acid sequence of human PGC-1α mRNA (Genbank Accession No. NM_{—}013261; SEQ ID NO: 12).

FIG. 23 is the amino acid sequence of human PGC-10 polypeptide (Genbank Accession No. NP_{—}573570; SEQ ID NO: 13).

FIG. 24 is the nucleic acid sequence of human PGC-1β mRNA (Genbank Accession No. NM_{—}133263; SEQ ID NO: 14).

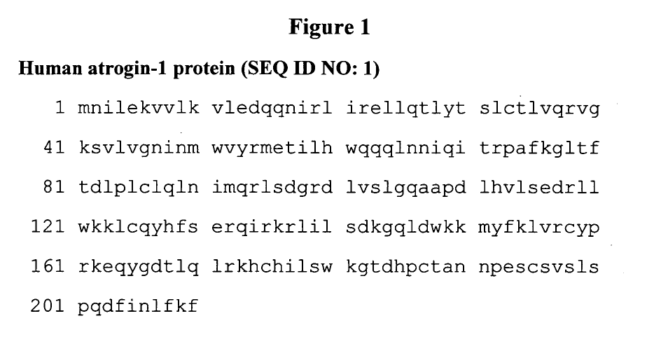

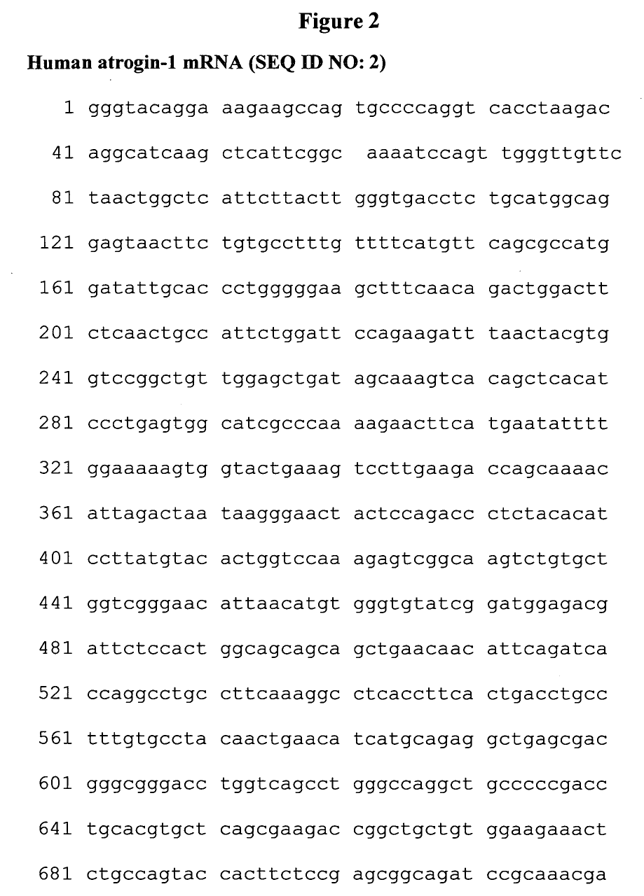

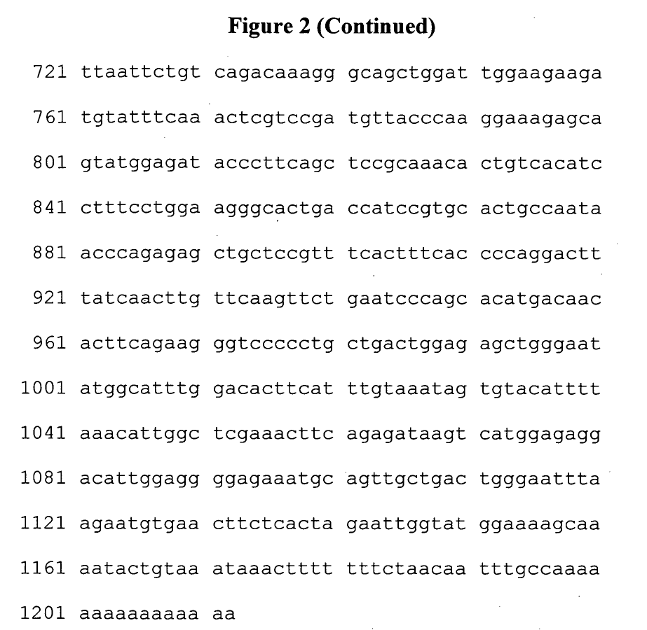

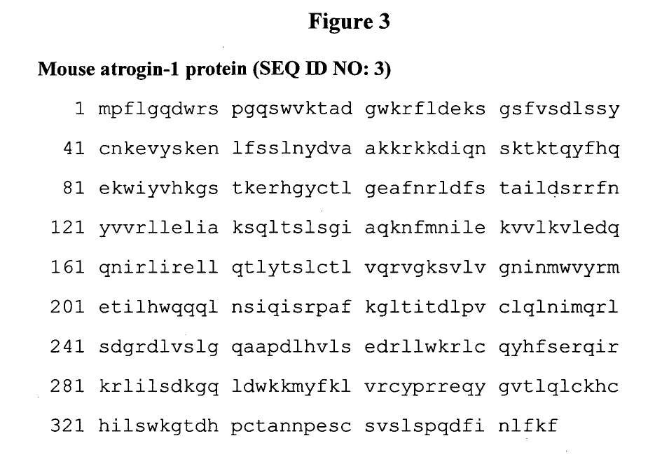

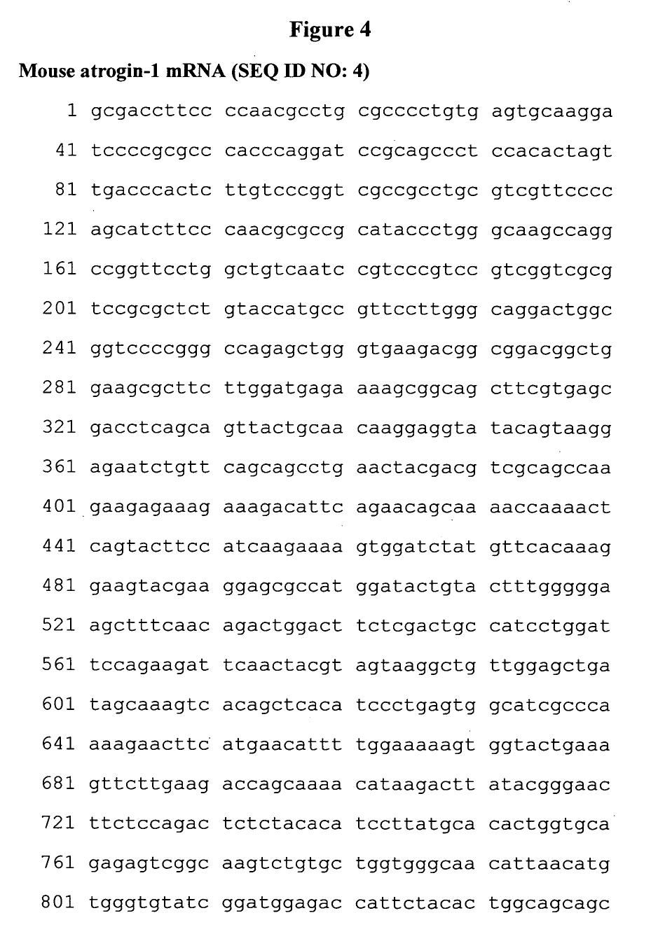

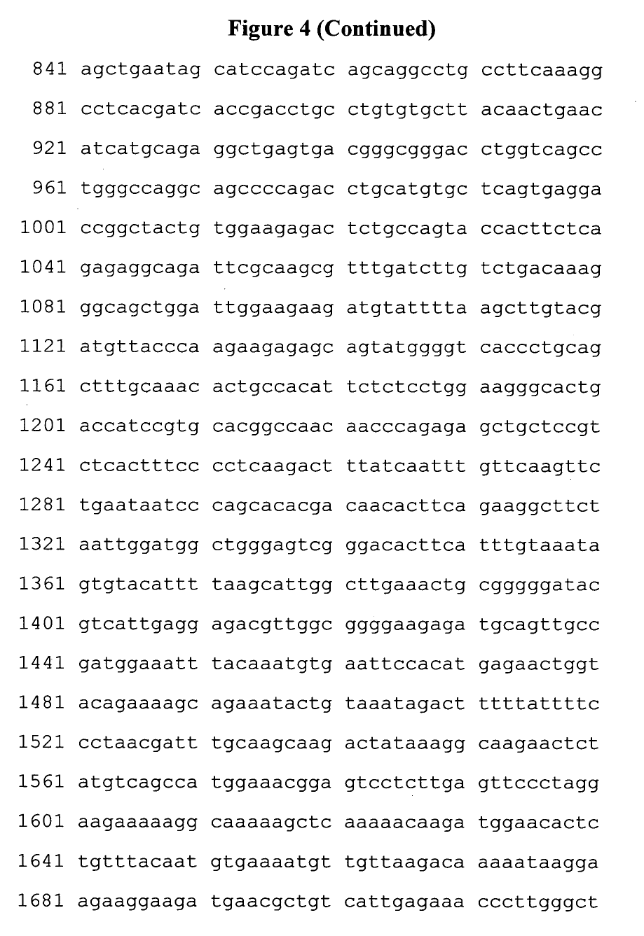

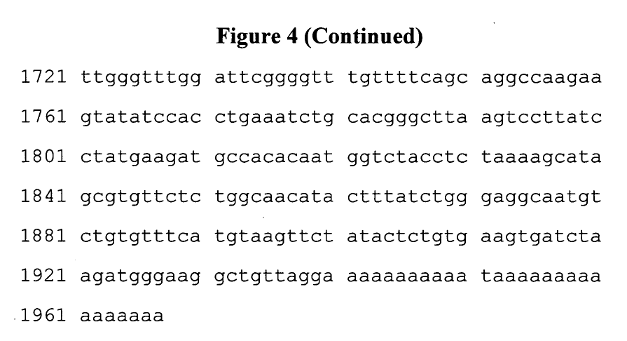

### DETAILED DESCRIPTION

We have discovered that atrogin-1, an E3 ubiquitn ligase, is upregulated during statin therapy or treatment. Specifically, we have discovered that the atrogin-1 protein is upregulated in cultured myocytes in the presence of statins and that this upregulation of atrogin-1 protein is time and dose dependent. Furthermore, statins induce myocyte dysfunction as myotubes are improperly formed in the presence of statins. This phenomenon is conserved throughout various species, as murine primary myocyte cell cultures demonstrate the same pattern of a) dose and time dependent upregulation of atrogin-1 in response to statins and b) improper myotube formation in the presence of statins. Based on these results we have discovered that atrogin-1 polypeptides and nucleic acid molecules can be used to diagnose or monitor statin-mediated myopathy. We have further shown that zebra fish somite development is dramatically altered by the presence of statins in the immediate environment (e.g., water). Further, using the zebrafish model, we have shown that the myopathic phenotype can be rescued using a morpholino directed to the atrogin-1 gene. Using a mouse model which lacks atrogin-1, we have also shown that in the absence of atrogin-1, statin-induced muscle damage does not occur or is vastly diminished. Taken together, these results support the critical role for atrogin-1 in the pathogenesis of statin-mediated myopathy.

Accordingly, the present invention features the use of atrogin-1 inhibitor compounds for treating or preventing a statin-mediated myopathy. The invention also feature methods for identifying patients at risk for developing a statin-mediated myopathy, including evaluating statins for their potential to induce a statin mediated myopathy in a subject undergoing or preparing to undergo statin therapy. The invention further features diagnostic and therapeutic monitoring methods that include the use of atrogin-1 nucleic acid molecules, polypeptides, and antibodies for the diagnosis and monitoring of a statin-mediated myopathy.

### Therapeutic Methods

Until now, the mechanisms underlying statin-mediated myopathy have remained controversial and poorly understood. We have shown a direct causal link between induction of muscle cell dysfunction and induction of the atrophy program by statin treatment via the ubiquitin-ligase atrogin-1. We have therefore developed atrogin-1 inhibitor compounds for the treatment or prevention of a statin-mediated myopathy. Examples of statin-mediated disorders that can be treated or prevented by the current invention are provided below.

### Statin-Mediated Myopathy

The advent of the HMG-CoA reductase inhibitors, or statins, in the 1980's as highly efficacious agents for the lowering of low-density lipoprotein-cholesterol (LDL-C) revolutionized treatment of hypercholesterolemia, a long established risk factor for premature coronary heart disease. Statins now marketed in the United States include altace (Ramipril), atorvastatin (Lipitor), fluvastatin (Lescol), lovastatin (Mevacor), pravastatin (Pravachol), simvastatin (Zocor), rosuvastatin, (Crestor), or pitavastatin. Additionally, there are other statins, some in clinical trials, including compactin, mevinolin, mevastatin, velostatin, synvinolin, or rivastatin (sodium 7-(4-fluorophenyl)2,6-diisopropyl-5-methoxymethylpyridin-3-yl)3,5-dihydroxy-6-heptanoate). Statins are well tolerated by most patients but can produce a variety of muscle-related complications or myopathies. The most serious risk of these is myositis with rhabdomyolysis. This risk has been emphasized by the withdrawal of cerivastatin in August 2001 after the drug was associated with approximately 100 rhabdomyolysis-related deaths. Rhabdomyolysis was also a factor in the withdrawal of the antihypertensive drug mibefradil in June 1998 and in the decision by Merck & Co. to abandon the development of a 160-mg sustained-release simvastatin formulation in the mid-1990s.

Myopathy can refer to any muscular disease, and here we differentiate myalgia as muscle ache or weakness in the absence of elevation in creatine kinase (CK), and myositis as adverse muscular symptoms associated with inflammation with and without increased CK levels. Rhabdomyolysis is a severe form of myositis involving myoglobulinuria, which can engender acute renal failure. Although rhabdomyolysis associated with statin treatment is rare, muscular pain and weakness are more frequent and may affect 7% of patients on statin monotherapy, with myalgia contributing up to 25% of all adverse events associated with statin use (Ucar et al., “HMG-CoA reductase inhibitors and myotoxicity,” Drug Safety 22:441-457, 2000). The effects of subclinical muscular side effects should not be underestimated, however, as they reduce patient compliance with possible discontinuation of therapy, limit physical activity, reduce the quality of life, and most importantly, may ultimately deprive the dyslipidemic patient at high risk for cardiovascular disease of the clinical benefit of statin treatment. Such myopathies become especially pertinent in the context of recent clinical trials, which have validated optimized reduction of morbi-mortality in cardiovascular disease using high-dose statin therapy. This is particularly relevant as increased statin dosage is closely associated with increased risk of muscular side effects. Furthermore, select patient populations require closer surveillance for statin myopathy, as their risk profile is increased. This includes advanced age (>80 years old); small frame; multisystem illness including diabetes; patients in the perioperative period; and concomitant medications. All statin new drug applications (NDAs) suggest that statin myotoxicity is dose dependent with a threshold effect at which risks exceed benefits—especially with regard to cerivastatin.

### Treatment of Statin-Mediated Myopathy

We have discovered that statins upregulate atrogin-1 expression in myocytes and this is associated with myocyte dysfunction and improper myotube formation. Any of the atrogin-1 inhibitor compounds described may be used for the treatment or prevention of a statin-mediated myopathy.

Statin-mediated myopathies can be diagnosed using the methods described herein in combination with techniques known in the art (e.g., muscle biopsy and evaluation of atrogin-1 products). Likewise, the therapeutic effectiveness of atrogin-1 inhibitor compounds can be measured using the above described in vitro and in vivo assays and methodology. Assays include any of the assays for atrogin-1 biological activity or expression as described herein wherein a compound that reduces or inhibits atrogin-1 biological activity is considered a compound useful for the treatment or prevention of a statin-mediated myopathy. Assays of atrogin-1 activity include for example, ubiquitination assays, calcineurin activity assays, substrate binding assays, and nuclear translocation assays. These assays and evaluation methods can be performed alone, or in combination with other assay techniques evaluating overall muscle health, including CK enzymatic assays.

### Atrogin-1 Inhibitor Compounds

We have discovered that atrogin-1 levels in myocytes are increased and that myocytes are dysfunctional in the presence of statins. Specifically, myotubes do not form properly and zebrafish somite development is dramatically affected in the presence of statins. Therefore, the invention features atrogin-1 inhibitor compounds for the treatment or prevention of a statin-mediated myopathy.

Atrogin-1 inhibitor compounds useful in the methods of the invention include any compound that can reduce or inhibit the biological activity or expression level of atrogin-1. Exemplary compounds include, but are not limited to, fragments of atrogin-1 (e.g., dominant negative fragments or fragments that are incapable of ubiquitin ligase activity, unable to bind substrate, or unable to undergo nuclear translocation); peptidyl or non-peptidyl compounds that specifically bind atrogin-1 (e.g., antibodies or antigen-binding fragments thereof), for example, at the ubiquitin ligase domain, substrate binding domain of atrogin-1, or N- or C-terminal nuclear translocation sequence (amino acids 62-66 and amino acids 267-288 of atrogin-1) and block atrogin-1 function; antisense nucleobase oligomers; morpholino oligonucleotides (e.g., SEQ ID NO: 8 or SEQ ID NO: 9, or those molecules which target the translation start sequence or splicing sequence of atrogin-1 mRNA); small RNAs; small molecule inhibitors; compounds that decrease the half-life of atrogin-1 mRNA or protein; compounds that decrease transcription or translation of atrogin-1; compounds that reduce or inhibit the expression levels of atrogin-1 polypeptides or decrease the biological activity of atrogin-1 polypeptides (e.g., PGC-1α or PGC-1β); compounds that increase the expression or biological activity of a second atrogin-1 inhibitor (e.g., a molecule which increases the transcription or translation of PGC-1α or PGC-1β protein (such as metformin) or an inhibitor that blocks atrogin-1 substrate binding or ubiquitin ligase activity); compounds that alter expression or biological activity of proteins upstream of atrogin-1 (e.g., phosphorylation of Foxo transcription factors including Foxo-1 and Foxo-3, and phosphorylation of PI3K/Akt or other Foxo-associated kinases); compounds that alter expression or biological activity of proteins downstream of atrogin-1 (e.g., Skp1, Cul1, Roc1, calcineurin A, or others); compounds that block atrogin-1-mediated downstream activities (e.g., ubiquitination, binding to SCF family members, inhibition of calcineurin A activity, and nuclear translocation); and compounds which alter expression or biological activity of other atrogin-1 associated proteins, or atrogenes, including MurF-1.

Preferred atrogin-1 inhibitor compounds will reduce or inhibit atrogin-1 biological activity or expression levels by at least 10%, 25%, 30%, 40%, 50%, 60%, 70%, 80%, 90%, 95%, 96%, 97%, 98%, 99%, or more. Preferably, the atrogin-1 compound can reduce or inhibit myocyte and/or myotube dysfunction, somite developmental irregularities, increased atrogin-1-specific ubiquitination of muscle proteins, atrogin-1-specific substrate binding of muscle proteins, and symptoms of a myopathy or statin-mediated myopathy, including myalgias, myalagia with associated creatine kinase elevations, myositis, or rhabdomyolysis by at least 10%, 20%, 25%, 30%, 40%, 50%, 60%, 70%, 80%, 90%, 95%, 96%, 97%, 98%, 99%, or more.

### Polypeptides

Polypeptides that specifically bind to atrogin-1 and reduce or inhibit the biological activity of atrogin-1 are included in the invention and can be used in the methods and compositions of the invention that require atrogin-1 inhibitor compounds. Preferred polypeptides include dominant negative fragments of atrogin-1 or polypeptides that bind to functional regions of the atrogin-1 protein, for example, the ubiquitin ligase domain or the substrate-binding domain. By binding to the functional domain, the polypeptide can inhibit the activity of atrogin-1, presumably by steric interference. An example of an atrogin-1 inhibitor compound is an atrogin-1 polypeptide lacking the N- and/or C-terminal nuclear localization sequence (ANLS atrogin-1) or having a mutation in a nuclear localization sequence (e.g., the N- and/or C-terminal nuclear localization sequences).

Any polypeptide that is used as an antagonist compound can be produced, purified, and/or modified using any of the methods and modifications known in the art or described herein. Examples of modifications which can be made to a polypeptide which is an atrogin-1 antagonist, include phosphorylation, acylation, glycosylation, pegylation (e.g., addition of polyethylene glycol), sulfation, prenylation, methylation, hydroxylation, carboxylation, and amidation.

The ability of any of the above polypeptides to function as an atrogin-1 inhibitor compound may be tested according to any of the assays described in the Examples.

### Antibodies

Antibodies that specifically bind to atrogin-1, have a high affinity (K_{D}<500 nM) for atrogin-1, and/or neutralize or prevent atrogin-1 activity are useful in the therapeutic methods of the invention. In one embodiment, the antibody, or fragment or derivative thereof, binds to the ubiquitin ligase domain or substrate binding domain of atrogin-1. One example of a polyclonal antibody specific for atrogin-1 is shown in the Examples below. The present invention includes, without limitation, anti-atrogin-1 monoclonal, polyclonal, chimeric, and humanized antibodies and functional equivalents or derivatives of antibodies as described below.

Pharmaceutical compositions, for example, including excipients, of any antibodies of the invention are also included. Methods for the preparation and use of antibodies for therapeutic purposes are described in several patents including U.S. Pat. Nos. 6,054,297; 5,821,337; 6,365,157; and 6,165,464 and are incorporated herein by reference. Antibodies can be polyclonal or monoclonal; monoclonal antibodies are preferred.

### Monoclonal and Polyclonal Antibodies

Methods for the generation of both monoclonal or polyclonal anti-atrogin-1 antibodies may be produced by methods known in the art. These methods include the immunological method described by Kohler and Milstein (Nature, 256: 495-497, 1975), Kohler and Milstein (Eur. J. Immunol, 6, 511-519, 1976), and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science, 246, 1275-1281, 1989).

Human antibodies can also be produced using phage display libraries (Marks et al., J. Mol. Biol., 222:581-597, 1991 and Winter et al. Annu. Rev. Immunol., 12:433-455, 1994). The techniques of Cole et al. and Boerner et al. are also useful for the preparation of human monoclonal antibodies (Cole et al., supra; Boerner et al., J. Immunol., 147: 86-95, 1991).

Monoclonal antibodies are isolated and purified using standard art-known methods. For example, antibodies can be screened using standard art-known methods such as ELISA against an atrogin-1 polypeptide or fragment or Western blot analysis. Non-limiting examples of such techniques are described in Examples II and III of U.S. Pat. No. 6,365,157, herein incorporated by reference.

The antibody may be prepared in any mammal, including mice, rats, rabbits, goats, and humans. The antibody may be a member of one of the following immunoglobulin classes: IgG, IgM, IgA, IgD, or IgE, and the subclasses thereof, and preferably is an IgG antibody.

While the preferred animal for producing monoclonal antibodies is mouse, the invention is not so limited; in fact, human antibodies may be used and may prove to be preferable. Such antibodies can be obtained by using human hybridomas (Cole et al., “Monoclonal Antibodies and Cancer Therapy”, Alan R. Liss Inc., p. 77-96, 1985).

Monoclonal antibodies, particularly those derived from rodents including mice, have been used for the treatment of various diseases; however, there are limitations to their use including the induction of a human anti-mouse immunoglobulin response that causes rapid clearance and a reduction in the efficacy of the treatment. For example, a major limitation in the clinical use of rodent monoclonal antibodies is an anti-globulin response during therapy (Miller et al., Blood, 62:988-995 1983; Schroff et al., Cancer Res., 45:879-885, 1985).

### Chimeric Antibodies

The art has attempted to overcome the problem of rodent antibody-induced anti-globulin response by constructing “chimeric” antibodies in which an animal antigen-binding variable domain is coupled to a human constant domain (U.S. Pat. No. 4,816,567; Morrison et al., Proc. Natl. Acad. Sci. USA, 81:6851-6855, 1984; Boulianne et al., Nature, 312:643-646, 1984; Neuberger et al., Nature, 314:268-270, 1985). Chimerized antibodies preferably have constant regions derived substantially or exclusively from human antibody constant regions and variable regions derived substantially or exclusively from the sequence of the variable region from a mammal other than a human. In the present invention, techniques developed for the production of chimeric antibodies by splicing the genes from a mouse antibody molecule of appropriate antigen specificity together with genes from a human antibody molecule can be used (Morrison et al., Proc. Natl. Acad. Sci. 81, 6851-6855, 1984; Neuberger et al., Nature 312, 604-608, 1984; Takeda et al., Nature 314, 452-454, 1985).

DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the CDRs derived substantially or exclusively from the corresponding human antibody regions and DNA encoding CDRs derived substantially or exclusively from a mammal other than a human.

Suitable sources of DNA molecules that encode fragments of antibodies include cells, such as hybridomas, that express the full-length antibody. The fragments may be used by themselves as antibody equivalents, or may be recombined into equivalents, as described above. The DNA deletions and recombinations described in this section may be carried out by known methods, such as those described in the published patent applications listed above.

### Humanized Antibodies

Humanized antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Methods for humanizing non-human antibodies are well known in the art (for reviews see Vaswani and Hamilton, Ann. Allergy Asthma Immunol., 81:105-119, 1998 and Carter, Nature Reviews Cancer, 1:118-129, 2001). Generally, a humanized antibody has one or more amino acid residues introduced into it from a source that is non-human. These non-human amino acid residues are often referred to as import residues, which are typically taken from an import variable domain.

Humanization of an antibody can be essentially performed following the methods known in the art (Jones et al., Nature, 321:522-525, 1986; Riechmann et al., Nature, 332:323-329, 1988; and Verhoeyen et al., Science, 239:1534-1536 1988), by substituting rodent CDRs or other CDR sequences for the corresponding sequences of a human antibody. Accordingly, such humanized antibodies are chimeric antibodies wherein substantially less than an intact human variable domain has been substituted by the corresponding sequence from a non-human species (see for example, U.S. Pat. No. 4,816,567). In practice, humanized antibodies are typically human antibodies in which some CDR residues and possibly some frameword residues are substituted by residues from analogous sites in rodent antibodies (Presta, Curr. Op. Struct. Biol., 2:593-596, 1992).

Additional methods for the preparation of humanized antibodies can be found in U.S. Pat. Nos. 5,821,337, and 6,054,297, and Carter, (supra) which are all incorporated herein by reference. The humanized antibody is selected from any class of immunoglobulins, including IgM, IgG, IgD, IgA and IgE, and any isotype, including IgG₁, IgG₂, IgG₃, and IgG₄. Where cytotoxic activity is not needed, such as in the present invention, the constant domain is preferably of the IgG₂ class. The humanized antibody may comprise sequences from more than one class or isotype, and selecting particular constant domains to optimize desired effector functions is within the ordinary skill in the art.

### Functional Equivalents or Derivatives of Antibodies

The invention also includes functional equivalents or derivatives of the antibodies described in this specification. Functional equivalents or derivatives include polypeptides with amino acid sequences substantially identical to the amino acid sequence of the variable or hypervariable regions of the antibodies of the invention. Functional equivalents have binding characteristics comparable to those of the antibodies, and include, for example, chimerized, humanized and single chain antibodies, antibody fragments, and antibodies, or fragments thereof, fused to a second protein, or fragment thereof. Methods of producing such functional equivalents are disclosed, for example, in PCT Publication No. WO93/21319; European Patent Application No. 239,400; PCT Publication No. WO89/09622; European Patent Application No. 338,745; European Patent Application No. 332424; and U.S. Pat. No. 4,816,567; each of which is herein incorporated by reference.

Functional equivalents of antibodies also include single-chain antibody fragments, also known as single-chain antibodies (scFvs). Single-chain antibody fragments are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Functional equivalents further include fragments of antibodies that have the same or comparable binding characteristics to those of the whole antibody. Such fragments may contain one or both Fab fragments or the F(ab′)₂ fragment. Preferably the antibody fragments contain all six CDRs of the whole antibody, although fragments containing fewer than all of such regions, such as three, four or five CDRs, are also functional.

Further, the functional equivalents may be or may combine members of any one of the following immunoglobulin classes: IgG, IgM, IgA, IgD, or IgE, and the subclasses thereof.

Equivalents of antibodies are prepared by methods known in the art. For example, fragments of antibodies may be prepared enzymatically from whole antibodies. Preferably, equivalents of antibodies are prepared from DNA encoding such equivalents. DNA encoding fragments of antibodies may be prepared by deleting all but the desired portion of the DNA that encodes the full-length antibody.

### Nucleic Acid Molecules

The present invention features nucleic acid molecules capable of binding atrogin-1 nucleic acids or polypeptides; mediating downregulation of the expression of an atrogin-1 polypeptide or nucleic acid; or mediating a decrease in the activity of a atrogin-1 polypeptide. Examples of the nucleic acids of the invention include, without limitation, antisense oligomers (e.g., morpholinos), dsRNAs (e.g., siRNAs and shRNAs), and aptamers.

### Antisense Oligomers

The present invention features antisense nucleobase oligomers to atrogin-1 and the use of such oligomers to downregulate expression of atrogin-1 mRNA. By binding to the complementary nucleic acid sequence (the sense or coding strand), antisense nucleobase oligomers are able to inhibit protein expression presumably through the enzymatic cleavage of the RNA strand by RNAse H. Preferably the antisense nucleobase oligomer is capable of reducing atrogin-1 protein expression in a cell that expresses increased levels of atrogin-1. Preferably the decrease in atrogin-1 protein expression is at least 10% relative to cells treated with a control oligonucleotide, preferably 20% or greater, more preferably 40%, 50%, 60%, 70%, 80%, 90% or greater. Methods for selecting and preparing antisense nucleobase oligomers are well known in the art. Methods for assaying levels of protein expression are also well known in the art and include Western blotting, immunoprecipitation, and ELISA.

One example of an antisense nucleobase oligomer particularly useful in the methods and compositions of the invention is a morpholino oligomer. Morpholinos are used to block access of other molecules to specific sequences within nucleic acid molecules. They can block access of other molecules to small (˜25 base) regions of ribonucleic acid (RNA). Morpholinos are sometimes referred to as PMO, an acronym for phosphorodiamidate morpholino oligo.

Morpholinos are used to knock down gene function by preventing cells from making a targeted protein or by modifying the splicing of pre-mRNA. Morpholinos are synthetic molecules that bind to complementary sequences of RNA by standard nucleic acid base-pairing. While morpholinos have standard nucleic acid bases, those bases are bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so morpholinos in organisms or cells are uncharged molecules.

Morpholinos act by “steric blocking” or binding to a target sequence within an RNA and blocking molecules which might otherwise interact with the RNA. Because of their completely unnatural backbones, morpholinos are not recognized by cellular proteins. Nucleases do not degrade morpholinos and morpholinos do not activate toll-like receptors and so they do not activate innate immune responses such as the interferon system or the NF-κB-mediated inflammation response. Morpholinos are also not known to modify methylation of DNA. Therefore, morpholinos directed to any part of atrogin-1 and that reduce or inhibit the expression levels or biological activity of atrogin-1 are particularly useful in the methods and compositions of the invention that require the use of atrogin-1 inhibitor compounds. For example, morpholinos may be targeted to both the coding and non-coding sequences of an mRNA (e.g., atrogin-1 mRNA). In preferred embodiments, the morpholinos may be designed to target the ATG or translation start site or a intron/exon splice site within the sequence of an mRNA (e.g., atrogin-1 mRNA). Two examples of morpholinos that target atrogin-1 mRNA are 5′-TTG TCC AAG AAA CGG CAT TGT CAA G-3′ (SEQ ID NO: 8) and 5′-AAA GCC ACC ATC ATG TAC CTG TCT G-3′ (SEQ ID NO: 9).

dsRNAs

The present invention also features the use of double stranded RNAs including, but not limited to siRNAs and shRNAs. Short double-stranded RNAs may be used to perform RNA interference (RNAi) to inhibit expression of atrogin-1. RNAi is a form of post-transcriptional gene silencing initiated by the introduction of double-stranded RNA (dsRNA). Short 15 to 32 nucleotide double-stranded RNAs, known generally as “siRNAs,” “small RNAs,” or “microRNAs” are effective at down-regulating gene expression in nematodes (Zamore et al., Cell 101: 25-33) and in mammalian tissue culture cell lines 20. (Elbashir et al., Nature 411:494-498, 2001). The further therapeutic effectiveness of this approach in mammals was demonstrated in vivo by McCaffrey et al. (Nature 418:38-39.2002). The small RNAs are at least 15 nucleotides, preferably, 17, 18, 19, 20, 21, 22, 23, 24, 25, 26, 27, 28, 29, 30, 31, 32, 33, 34, 35, nucleotides in length and even up to 50 or 100 nucleotides in length (inclusive of all integers in between). Such small RNAs that are substantially identical to or complementary to any region of atrogin-1, are included in the invention. Non-limiting examples of desirable small RNAs are substantially identical (e.g., 60%, 65%, 70%, 75%, 80%, 85%, 90%, 95%, 96%, 97%, 98%, 99%, or 100% sequence identity) to or complementary to the atrogin-1 translational start sequence or the splicing sequence. For example, the sequence 5′-TTG TCC AAG AAA CGG CAT TGT CAA G-3′ (SEQ ID NO: 8) may be used for targeting the z-atrogin-1 translational start site and the sequence 5′-AAA GCC ACC ATC ATG TAC CTG TCT G-3′ (SEQ ID NO: 9) may be used to target the z-atrogin-1 splicing sequence.

The invention includes any small RNA substantially identical to at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between) of any region of atrogin-1 (e.g., SEQ ID NO: 1 and SEQ ID NO: 3). It should be noted that longer dsRNA fragments can be used that are processed into such small RNAs. Useful small RNAs can be identified by their ability to decrease atrogin-lexpression levels or biological activity. Small RNAs can also include short hairpin RNAs in which both strands of an siRNA duplex are included within a single RNA molecule.

The specific requirements and modifications of small RNA are known in the art and are described, for example, in PCT Publication No. WO01/75164, and U.S. Application Publication Nos. 20060134787, 20050153918, 20050058982, 20050037988, and 20040203145, the relevant portions of which are herein incorporated by reference. In particular embodiments, siRNAs can be synthesized or generated by processing longer double-stranded RNAs, for example, in the presence of the enzyme dicer under conditions in which the dsRNA is processed to RNA molecules of about 17 to about 26 nucleotides. siRNAs can also be generated by expression of the corresponding DNA fragment (e.g., a hairpin DNA construct). Generally, the siRNA has a characteristic 2- to 3-nucleotide 3′ overhanging ends, preferably these are (2′-deoxy) thymidine or uracil. The siRNAs typically comprise a 3′ hydroxyl group. In some embodiments, single stranded siRNAs or blunt ended dsRNA are used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs, e.g., substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA molecules can be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using standard procedures for in vitro transcription of RNA and dsRNA annealing procedures such as those described in Elbashir et al. (Genes & Dev., 15:188-200, 2001), Girard et al. (Nature 442:199-202, 2006), Aravin et al. (Nature 442:203-207, 2006), Grivna et al. (Genes Dev. 20:1709-1714, 2006), and Lau et al. (Science 313:305-306, 2006). siRNAs are also obtained by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g., size exclusion chromatography), glycerol gradient centrifugation, and affinity purification with antibody can be used to isolate the small RNAs.

Short hairpin RNAs (shRNAs), as described in Yu et al. (Proc. Natl. Acad. Sci. USA, 99:6047-6052, 2002) or Paddison et al. (Genes & Dev, 16:948-958, 2002), incorporated herein by reference, can also be used in the methods of the invention. shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

A variety of methods are available for transfection, or introduction, of dsRNA into mammalian cells. For example, there are several commercially available transfection reagents useful for lipid-based transfection of siRNAs including but not limited to: TransIT-TKO™ (Mirus, Cat. # MIR 2150), Transmessenger™ (Qiagen, Cat. # 301525), Oligofectamine™ and Lipofectamine™ (Invitrogen, Cat. # MIR 12252-011 and Cat. #13778-075), siPORT™ (Ambion, Cat. #1631), DharmaFECT™ (Fisher Scientific, Cat. # T-2001-01). Agents are also commercially available for electroporation-based methods for transfection of siRNA, such as siPORTer™ (Ambion Inc. Cat. # 1629). Microinjection techniques can also be used. The small RNA can also be transcribed from an expression construct introduced into the cells, where the expression construct includes a coding sequence for transcribing the small RNA operably linked to one or more transcriptional regulatory sequences. Where desired, plasmids, vectors, or viral vectors can also be used for the delivery of dsRNA or siRNA and such vectors are known in the art. Protocols for each transfection reagent are available from the manufacturer. Additional methods are known in the art and are described, for example in U.S. Patent Application Publication No. 20060058255.

### Aptamers

The present invention also features aptamers to atrogin-1 and the use of such aptamers to downregulate expression of atrogin-1 proteins or atrogin-1 mRNA. Aptamers are nucleic acid molecules that form tertiary structures that specifically bind to a target molecule, such as an atrogin-1 polypeptide. The generation and therapeutic use of aptamers are well established in the art. See, e.g., U.S. Pat. No. 5,475,096. For example, an atrogin-1 aptamer may be a pegylated modified oligonucleotide, which adopts a three-dimensional conformation that enables it to bind to atrogin-1. Additional information on aptamers can be found, for e.g., in U.S. Patent Application Publication No. 20060148748.

### Therapeutic Formulations

Statin compounds for use in the methods are pharmaceutical formulations of the HMG-CoA reductase inhibitors understood to be those active agents which may be used to lower the lipid levels including cholesterol in blood. The class of HMG-CoA reductase inhibitors comprises compounds having differing structural features. For example, HMG-CoA reductase inhibitors include: atorvastatin, cerivastatin, fluvastatin, lovastatin, pitavastatin (formerly itavastatin), pravastatin, rosuvastatin, and simvastatin, or, in each case, a pharmaceutically acceptable salt thereof (e.g., a calcium salt). Preferred HMG-CoA reductase inhibitors are those agents which have been marketed as lipid lowering compounds, most preferred is fluvastatin, atorvastatin, pitavastatin or simvastatin, or a pharmaceutically acceptable salt thereof.

The dosage of the active compound can depend on a variety of factors, such as mode of administration, homeothermic species, age and/or individual condition. Statins may be administered at a dosage of generally between about 1 and about 500 mg/day, more preferably from about 1 to about 40, 50, 60, 70 or 80 mg/day, advantageously from about 20 to about 40 mg per day. For example, tablets or capsules comprising, e.g., from about 5 mg to about 120 mg, preferably, when using fluvastatin, for example, 20 mg, 40 mg, or 80 mg (equivalent to the free acid) of fluvastatin, for example, administered once a day.

The invention includes the use of atrogin-1 inhibitor compounds to treat, prevent or reduce a statin-mediated myopathy in a subject. The atrogin-1 inhibitor compound can be administered at anytime, for example, after diagnosis or detection of a statin-mediated myopathy, or for prevention of a statin-mediated myopathy in subjects that have not yet been diagnosed with a statin-mediated myopathy but are at risk of developing such a disorder, or after a risk of developing a statin-mediated myopathy is determined. An atrogin-1 inhibitor compound may also be administered simultaneously with a statin. An atrogin-1 inhibitor compound of the invention may be formulated with a pharmaceutically-acceptable diluent, carrier, or excipient, in unit dosage form. Conventional pharmaceutical practice may be employed to provide suitable formulations or compositions to administer atrogin-1 inhibitor compound of the invention to patients suffering from a statin-mediated myopathy. Administration may begin before the patient is symptomatic. The atrogin-1 inhibitor compound of the present invention can be formulated and administered in a variety of ways, e.g., those routes known for specific indications, including, but not limited to, topically, orally, subcutaneously, bronchioscopic injection, intravenously, intracerebrally, intranasally, transdermally, intraperitoneally, intramuscularly, intrapulmonary, vaginally, rectally, intraarterially, intralesionally, parenterally, intraventricularly in the brain, or intraocularly. The atrogin-1 inhibitor compound can be in the form of a pill, tablet, capsule, liquid, or sustained release tablet for oral administration; or a liquid for intravenous administration, subcutaneous administration, or injection; for intranasal formulations, in the form of powders, nasal drops, or aerosols; or a polymer or other sustained-release vehicle for local administration.

The invention also includes the use of atrogin-1 inhibitor compounds to treat, prevent or reduce a statin-mediated myopathy in a biological sample derived from a subject (e.g., treatment of a biological sample ex vivo) using any means of administration and formulation described herein. The biological sample to be treated ex vivo may include any biological fluid (e.g., blood, serum, plasma, or cerebrospinal fluid), cell (e.g., a myocyte), or tissue (e.g., muscle tissue) from a subject that has a statin-mediated myopathy or the propensity to develop a statin-induced myopathy. The biological sample treated ex vivo with the atrogin-1 inhibitor may be reintroduced back into the original subject or into a different subject. The ex vivo treatment of a biological sample with an atrogin-1 inhibitor, as described herein, may be repeated in an individual subject (e.g., at least once, twice, three times, four times, or at least ten times). Additionally, ex vivo treatment of a biological sample derived from a subject with an atrogin-1 inhibitor, as described herein, may be repeated at regular intervals (non-limiting examples include daily, weekly, monthly, twice a month, three times a month, four times a month, bi-monthly, once a year, twice a year, three times a year, four times a year, five times a year, six times a year, seven times a year, eight times a year, nine times a year, ten times a year, eleven times a year, and twelve times a year).

Therapeutic formulations are prepared using standard methods known in the art by mixing the active ingredient having the desired degree of purity with optional physiologically acceptable carriers, excipients or stabilizers (Remington's Pharmaceutical Sciences (20th edition), ed. A. Gennaro, 2000, Lippincott, Williams & Wilkins, Philadelphia, Pa.), in the form of lyophilized formulations or aqueous solutions. Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEENT™, PLURONICS™, or PEG. Optionally, but preferably, the formulation contains a pharmaceutically acceptable salt, preferably sodium chloride, and preferably at about physiological concentrations. The formulation may also contain the atrogin-1 inhibitor compound in the form of a calcium salt. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents.

For parenteral administration, the atrogin-1 inhibitor compound is formulated in a unit dosage injectable form (solution, suspension, emulsion) in association with a pharmaceutically acceptable parenteral vehicle. Such vehicles are inherently nontoxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives.

The dosage required depends on the choice of the route of administration; the nature of the formulation; the nature of the subject's illness; the subject's size, weight, surface area, age, and sex; other drugs being administered; and the judgment of the attending physician. Wide variations in the needed dosage are to be expected in view of the variety of polypeptides and fragments available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 6-, 8-, 10-, 20-, 50-, 100-, 150-, or more). Encapsulation of the polypeptide in a suitable delivery vehicle (e.g., polymeric microparticles or implantable devices) may increase the efficiency of delivery, particularly for oral delivery.

As described above, the dosage of the atrogin-1 inhibitor compound will depend on other clinical factors such as weight and condition of the subject and the route of administration of the compound. For treating subjects, between approximately 0.01 mg/kg to 500 mg/kg body weight of the atrogin-1 inhibitor compound can be administered. A more preferable range is 0.01 mg/kg to 50 mg/kg body weight with the most preferable range being from 1 mg/kg to 25 mg/kg body weight. Depending upon the half-life of the atrogin-1 inhibitor compound in the particular subject, the atrogin-1 inhibitor compound can be administered between several times per day to once a week. The methods of the present invention provide for single as well as multiple administrations, given either simultaneously or over an extended period of time.

Alternatively, a polynucleotide containing a nucleic acid sequence encoding an atrogin-1 inhibitor compound (e.g., an mRNA encoding PGC-1α protein) can be delivered to the appropriate cells in the subject. Expression of the coding sequence can be directed to any cell in the body of the subject, preferably a myocyte. This can be achieved, for example, through the use of polymeric, biodegradable microparticle or microcapsule delivery devices known in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome-mediated transfection, electroporation, calcium phosphate/DEAE dextran methods, gene gun, and microinjection.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of an atrogin-1 inhibitor compound.

Alternatively, tissue specific targeting can be achieved by the use of tissue- or cell-specific transcriptional regulatory elements which are known in the art (e.g., myocyte-specific promoters or enhancers). Delivery of “naked DNA” (i.e., without a delivery vehicle) to an intramuscular, intradermal, or subcutaneous site is another means to achieve in vivo expression.

Gene delivery using viral vectors such as adenoviral, retroviral, lentiviral, or adeno-asociated viral vectors can also be used. Numerous vectors useful for this purpose are generally known and have been described. In the relevant polynucleotides (e.g., expression vectors), the nucleic acid sequence encoding the atrogin-1 inhibitor polypeptide (including an initiator methionine and optionally a targeting sequence) is operatively linked to a promoter or enhancer-promoter combination. Short amino acid sequences can act as signals to direct proteins to specific intracellular compartments. Such signal sequences are described in detail in U.S. Pat. No. 5,827,516, incorporated herein by reference in its entirety. An ex vivo strategy can also be used for therapeutic applications. Ex vivo strategies involve transfecting or transducing cells obtained from the subject with a polynucleotide encoding an atrogin-1 inhibitor compound. The transfected or transduced cells are then returned to the subject. Such cells act as a source of the atrogin-1 inhibitor compound for as long as they survive in the subject.

Atrogin-1 inhibitor compound for use in the present invention may also be modified in a way to form a chimeric molecule comprising atrogin-1 inhibitor compound fused to another, heterologous polypeptide or amino acid sequence, such as an Fc sequence for stability.

The atrogin-1 inhibitor compound can be packaged alone or in combination with other therapeutic compounds as a kit (e.g., with a statin compound). Non-limiting examples include kits that contain, for example, two pills, a powder, a suppository and a liquid in a vial, or two topical creams. Desirably, a kit contains both a atrogin-1 inhibitor compound and a statin.

The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Combination therapies of the invention include this sequential administration, as well as administration of these therapeutic agents, in a substantially simultaneous manner. Substantially simultaneous administration can be accomplished, for example, by administering to the subject an atrogin-1 inhibitor compound and a statin in multiple capsules or injections. The components of the combination therapies, as noted above, can be administered by the same route or by different routes. For example, a statin compound and an atrogin-1 inhibitor compound may both be administered in the same way (e.g., via oral administration). In different embodiments, a statin compound may be administered by orally, while the other atrogin-1 inhibitor compounds may be administered intramuscularly, subcutaneously, topically or all therapeutic agents may be administered orally or all therapeutic agents may be administered by intravenous injection. The temporal sequence and or route of administration in which the therapeutic agents may depend upon the status of the patient. For example a patient at risk for developing a statin-mediated myopathy may begin receiving an administration or multiple administrations of an atrogin-1 inhibitor compound prior to administration of a statin compound, simultaneously to a statin compound, or consequent to administration of a statin compound. A patient diagnosed with a statin mediated myopathy may, for example, terminate administration of a statin compound, or receive administration of a different statin compound while beginning or maintaining administration of an atrogin-1 inhibitor compound. Likewise, monitoring a patient undergoing treatment for a statin mediated myopathy would likely dictate the temporal sequence and/or route of administration based on the efficacy of treatment as established by the clinician.

### Diagnostic Methods

The present invention features methods and compositions for the diagnosis of a statin-mediated myopathy or the propensity to develop such a condition using atrogin-1 polypeptides, nucleic acid molecules, and antibodies. The methods and compositions can include the measurement of atrogin-1 polypeptides, either free or bound to another molecule, or any fragments or derivatives thereof. Alterations in atrogin-1 expression or biological activity in a test sample as compared to a normal reference can be used to diagnose any of the disorders of the invention.

A subject having a statin-mediated myopathy, or a propensity to develop such a condition, will show an alteration (e.g., an increase or a decrease of 10%, 20%, 30%, 40%, 50%, 60%, 70%, 80%, 90%, or more) in the expression of an atrogin-1 polypeptide or an atrogin-1 biological activity. For example, an increase in the atrogin-1 polypeptide levels compared to a normal reference sample or level is diagnostic of a statin-mediated myopathy or a propensity to develop a statin-mediated myopathy. The atrogin-1 polypeptide can include full-length atrogin-1 polypeptide, degradation products, alternatively spliced isoforms of atrogin-1 polypeptide, enzymatic cleavage products of atrogin-1 polypeptide, atrogin-1 bound to a substrate or ligand, or free atrogin-1.

Standard methods may be used to measure levels of atrogin-1 polypeptide in any bodily fluid, including, but not limited to, urine, blood, serum, plasma, saliva, amniotic fluid, or cerebrospinal fluid. Such methods include immunoassay, ELISA, Western blotting using antibodies directed to atrogin-1 polypeptide, and quantitative enzyme immunoassay techniques. ELISA assays are the preferred method for measuring levels of atrogin-1 polypeptide. In one example, an atrogin-1 binding protein, for example an antibody that specifically binds a atrogin-1 polypeptide, is used in an immunoassay for the detection of atrogin-1 and the diagnosis of any of the disorders described herein or the identification of a subject at risk of developing such disorders.

The measurement of antibodies specific to atrogin-1 polypeptide in a patient may also be used for the diagnosis of a statin-mediated myopathy or the propensity to develop a statin-mediated myopathy. Antibodies specific to atrogin-1 polypeptide may be measured in any bodily fluid, including, but not limited to, 1 urine, blood, serum, plasma, saliva, amniotic fluid, or cerebrospinal fluid. ELISA assays are the preferred method for measuring levels of anti-atrogin-1 antibodies in a bodily fluid. An increased level of anti-atrogin-1 antibodies in a bodily fluid is indicative of a statin-induced myopathy or the propensity to develop a statin-mediated myopathy.

Atrogin-1 nucleic acid molecules, or fragments or oligonucleotides of atrogin-1 that hybridize to atrogin-1 at high stringency may be used as a probe to monitor expression of atrogin-1 nucleic acid molecules in the diagnostic methods of the invention. Any of the atrogin-1 nucleic acid molecules above can also be used to identify subjects having a genetic variation, mutation, or polymorphism in a atrogin-1 nucleic acid molecule that are indicative of a predisposition to develop the conditions. These polymorphisms may affect atrogin-1 nucleic acid or polypeptide expression levels or biological activity. Detection of genetic variation, mutation, or polymorphism relative to a normal, reference sample can be used as a diagnostic indicator of a subject likely to develop a statin-mediated myopathy while undergoing statin therapy, or the propensity to develop such a condition.

Such genetic alterations may be present in the promoter sequence, an open reading frame, intronic sequence, or untranslated 3′ region of a atrogin-1 gene. As noted throughout, specific alterations in the levels of biological activity of atrogin-1 can be correlated with the likelihood of a statin-mediated myopathy, or the predisposition to the same. As a result, one skilled in the art, having detected a given mutation, can then assay one or more metrics of the biological activity of the protein to determine if the mutation causes or increases the likelihood of a statin-mediated myopathy, or the predisposition to the same. For example, a patient may have a polymorphism in the promoter atrogin-1, which may increase the gene expression of atrogin-1. In such an instance, the polymorphism in the promoter may be used as a diagnostic tool for indentifying a patient with a statin-mediated myopathy or the propensity to develop a statin-mediated myopathy.

In one embodiment, a subject having a statin-mediated myopathy, or a predisposition to the same, will show an increase in the expression of a nucleic acid encoding atrogin-1. Methods for detecting such alterations are standard in the art and are described in Sandri et al. (Cell, 117:399-412, 2004). In one example Northern blotting or real-time PCR is used to detect atrogin-1 mRNA levels (Sandri et al., supra, and Bdolah et al., Am. J. Physio. Regul. Integre. Comp. Physiol. 292:R971-R976, 2007).

In another embodiment, hybridization at high stringency with PCR probes that are capable of detecting a atrogin-1 nucleic acid molecule, including genomic sequences, or closely related molecules, may be used to hybridize to a nucleic acid sequence derived from a subject having a statin-mediated myopathy, or at risk of developing such a disorder. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations in an atrogin-1 nucleic acid molecule, or may be used to monitor expression levels of a gene encoding an atrogin-1 polypeptide (Sandri et al., supra, and Bdolah et al., supra).

Another method of detecting atrogin-1 useful in the diagnostic methods of the invention includes the detection of antibodies that specifically bind to atrogin-1 in the blood or serum of a subject. For such a diagnostic methods, an atrogin-1 polypeptide, or fragment thereof, is used to detect the presence of atrogin-1 antibodies in the blood or serum of a subject. The subject sample can be compared to a reference, preferably a normal reference and an increase in the level of anti-atrogin-1 antibodies present is indicative of a statin-mediated myopathy.

Diagnostic methods can include measurement of absolute levels of atrogin-1 polypeptide, nucleic acid, or antibody, or relative levels of atrogin-1 polypeptide, nucleic acid, or antibody as compared to a reference sample. In one example, alterations in the levels of atrogin-1 polypeptide, nucleic acid, or antibody as compared to a normal reference, are considered a positive indicator of a statin-mediated myopathy, or the propensity to develop such a disorder (an increase in the levels is indicative of a statin-mediated myopathy).

In any of the diagnostic methods, the level of atrogin-1 polypeptide, nucleic acid, or antibody, or any combination thereof, can be measured at least two different times from the same subject and an alteration in the levels (e.g., by 10%, 20%, 30%, 40%, 50%, 60%, 70%, 80%, 90%, or more) over time is used as an indicator of a statin-mediated myopathy, or the propensity to develop such a condition. It will be understood by the skilled artisan that for diagnostic methods that include comparing of the atrogin-1 polypeptide, nucleic acid, or antibody level to a reference level, particularly a prior sample taken from the same subject, a change over time (e.g., an increase) with respect to the baseline level can be used as a diagnostic indicator of a statin-mediated myopathy, or a predisposition to either condition. The level of atrogin-1 polypeptide, nucleic acid, or antibody in the bodily fluids of a subject having a statin-mediated myopathy, or the propensity to develop such a condition may be altered, e.g., increased by as little as 10%, 20%, 30%, or 40%, or by as much as 50%, 60%, 70%, 80%, or 90% or more, relative to the level of atrogin-1 polypeptide, nucleic acid, or antibody in a prior sample or samples. The level of atrogin-1 polypeptide, nucleic acid, or antibody in the bodily fluids of a subject having a myopathy (e.g., a statin-induced myopathy), or the propensity to develop such a condition may be altered, e.g., decreased by as little as 10%, 20%, 30%, or 40%, or by as much as 50%, 60%, 70%, 80%, or 90% or more, relative to the level of atrogin-1 polypeptide, nucleic acid, or antibody in a prior sample or samples.

The diagnostic methods described herein can be used individually or in combination with any other diagnostic method described herein for a more accurate diagnosis of the presence of, severity of, or predisposition to a statin-mediated myopathy, or a predisposition to a statin-mediated myopathy.

### Diagnostic Kits

The invention also provides for a diagnostic test kit. For example, a diagnostic test kit can include polypeptides (e.g., antibodies that specifically bind to atrogin-1 polypeptide), and components for detecting, and more preferably evaluating binding between the polypeptide (e.g., antibody) and the atrogin-1 polypeptide. In another example, the kit can include an atrogin-1 polypeptide or fragment thereof for the detection of atrogin-1 antibodies in the serum or blood of a subject sample. For detection, either the antibody or the atrogin-1 polypeptide is labeled, and either the antibody or the atrogin-1 polypeptide is substrate-bound, such that the atrogin-1 polypeptide-antibody interaction can be established by determining the amount of label attached to the substrate following binding between the antibody and the atrogin-1 polypeptide. A conventional ELISA is a common, art-known method for detecting antibody-substrate interaction and can be provided with the kit of the invention. Atrogin-1 polypeptides can be detected in virtually any bodily fluid, such as urine, plasma, blood serum, semen, or cerebrospinal fluid. A kit that determines an alteration in the level of atrogin-1 polypeptide relative to a reference, such as the level present in a normal control, is useful as a diagnostic kit in the methods of the invention.

Desirably, the kit will contain instructions for the use of the kit. In one example, the kit contains instructions for the use of the kit for the diagnosis of a statin-mediated myopathy or a propensity to develop a statin-mediated myopathy. In yet another example, the kit contains instructions for the use of the kit to monitor therapeutic treatment or dosage regimens.

The kit can also contain a standard curve indicating levels of atrogin-1 that fall within the normal range and levels that would be considered diagnostic of a statin-mediated myopathy, or the propensity to develop any such disorder.

### Subject Monitoring

The diagnostic methods described herein can also be used to monitor a statin-mediated myopathy during therapy or to determine the dosages of therapeutic compounds. For example, alterations (e.g., a decrease as compared to the positive reference sample or level for a statin-mediated myopathy indicates an improvement in or the absence of statin-mediated myopathy). In this embodiment, the levels of atrogin-1 polypeptide, nucleic acid, or antibodies are measured repeatedly as a method of not only diagnosing disease but also monitoring the treatment, prevention, or management of the disease. In order to monitor the progression of a statin-mediated myopathy in a subject, subject samples are compared to reference samples taken early in the diagnosis of the disorder. Such monitoring may be useful, for example, in assessing the efficacy of a particular drug in a subject, determining dosages, or in assessing disease progression or status. For example, atrogin-1 levels can be monitored in a patient having a statin-mediated myopathy and as levels of atrogin-1 decrease, the dosage or administration of atrogin-1 inhibitor compounds may be decreased as well. In addition, the diagnostic methods of the invention can be used to monitor a subject that has risk factors indicative of a statin-mediated myopathy. For example, a subject having a family history of a cardiovascular disease controlled by statin-treatment with ensuing overt statin-mediated myopathic symptoms or the early indications for such a disorder (e.g., myalgia, myosits with or without CK elevation, rhabdomyolysis). In such an example, the therapeutic methods of the invention or those known in the art can then be used proactively to promote myocyte cell health and to prevent the disorder from developing or from developing further. In another example, a subject having a early indications of a statin-mediated myopathy (e.g., subclinical statin-mediated myopathic indicies, mild myalgia, myosits, no detectable CK elevation) can be treated with the therapeutic methods of the invention for statin-mediated myopathy to prevent progressive myopathic statin-induced disease.

### Screening Assays

As discussed above, we have discovered that atrogin-1 is a ubiquitin ligase with tissue specific expression that is critical for normal myocyte function and maintenance. Increases in atrogin-1 levels or biological activity results in increased cell catabolism and consequent breakdown of cellular proteins to mobilized amino acids; therefore, compounds that decrease the levels or biological activity of atrogin-1 are useful for treating statin-mediated myopathies. Based on these discoveries, atrogin-1 compositions of the invention are useful for the high-throughput low-cost screening of candidate compounds to identify those that modulate, alter, or decrease (e.g., by at least 10%, 20%, 30%, 40%, 50%, 60%, 70%, 80%, 90%, 95%, or more), the expression or biological activity of atrogin-1. Compounds that decrease the expression or biological activity of atrogin-1 can be used for the treatment or prevention of a statin-mediated myopahy.

Any number of methods are available for carrying out screening assays to identify new candidate compounds that modulate (e.g., decrease) the expression of an atrogin-1 polypeptide or nucleic acid molecule. In one working example, candidate compounds are added at varying concentrations to the culture medium of cultured cells expressing an atrogin-1 nucleic acid molecule. Gene expression is then measured, for example, by microarray analysis, Northern blot analysis (Ausubel et al., Current Protocols in Molecular Biology, Wiley Interscience, New York, 2001), or RT-PCR, using any appropriate fragment prepared from the atrogin-1 nucleic acid molecule as a hybridization probe. The level of gene expression in the presence of the candidate compound is compared to the level measured in a control culture medium lacking the candidate compound. A compound that promotes a decrease in the expression of an atrogin-1 gene or nucleic acid molecule, or a functional equivalent thereof, is considered useful in the invention. If the compound promotes a decrease in the levels of the atrogin-1 gene or nucleic acid molecule; such a compound may be used, for example, as a therapeutic to treat a statin-mediated myopathy.

Alternatively or additionally, statin compounds with lower risk of associated myopathy can be identified by adding candidate statin compounds to the culture medium of cells expressing atrogin-1. The level of atrogin-1 expression can then be compared to the level in cells treated with a statin with a known high risk of myopathy. A candidate statin compound that promotes a lower level of atrogin-1 expression as compared to the expression in cells treated with a known statin can be used as a therapeutic to treat a statin-mediated myopathy.

The zebrafish assays described herein (e.g., somite development in the presence of statins and ensuing developmental defects) are also useful assays for identifying atrogin-1 inhibitor compounds. For example, the evaluation of the altered somite development phenotype using an atrogin-1 inhibitor compound comprising a morpholino specific for the atrogin-1 is shown in the Examples.

In another working example, an atrogin-1 nucleic acid molecule is expressed as a transcriptional or translational fusion with a detectable reporter, and expressed in an isolated cell (e.g., mammalian or insect cell) under the control of a heterologous promoter, such as an inducible promoter. The cell expressing the fusion molecule is then contacted with a candidate compound, and the expression of the detectable reporter in that cell is compared to the expression of the detectable reporter in an untreated control cell. A candidate compound that decreases the expression of an atrogin-1 detectable reporter fusion is a compound that is useful as a therapeutic to promote myocyte cell health, and to treat, prevent or reduce symptoms of a statin-mediated myopathy in a subject.

In another working example, the effect of candidate compounds may be measured at the level of polypeptide expression using the same general approach and standard immunological techniques, such as Western blotting or immunoprecipitation with an antibody specific for an atrogin-1 polypeptide. For example, immunoassays may be used to detect or monitor the expression of atrogin-1 polypeptides in an organism. Polyclonal or monoclonal antibodies that are capable of binding to such a polypeptide may be used in any standard immunoassay format (e.g., ELISA, Western blot, or RIA assay) to measure the level of the polypeptide. In some embodiments, a compound that promotes an alteration, such as a decrease, in the expression or biological activity of an atrogin-1 polypeptide is considered particularly useful. A candidate compound that decreases the expression level or biological activity of an atrogin-1 polypeptide is a compound that is useful as a therapeutic to treat a statin-mediated myopathy.

In yet another working example, candidate compounds may be screened to identify those that specifically bind to an atrogin-1 polypeptide, preferably one that specifically binds to the ubiquitin-ligase domain or the substrate-binding domain. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind to an atrogin-1 polypeptide. Compounds that specifically bind to atrogin-1 and preferably act as an atrogin-1 inhibitor compound can be used for the treatment of a statin-mediated myopathy.

In one particular working example, a candidate compound that binds to an atrogin-1 polypeptide may be identified using a chromatography-based technique. For example, a recombinant atrogin-1 may be purified by standard techniques from cells engineered to express atrogin-1 and may be immobilized on a column. A solution of candidate compounds is then passed through the column, and a compound specific for the atrogin-1 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to function an inhibitor of the atrogin-1 polypeptide. Compounds isolated by this approach may also be used, for example, as therapeutics to treat or prevent a statin-mediated myopathy in a subject. Compounds that are identified as binding to atrogin-1 with an affinity constant less than or equal to 10 mM are considered particularly useful in the invention. Alternatively, any in vivo protein interaction detection system, for example, a two-hybrid assay, may be utilized to identify compounds or proteins that bind to an atrogin-1 polypeptide of the invention.

Atrogin-1 inhibitor compounds useful in the methods of the invention can be identified using any of the assays described above. Preferred atrogin-1 inhibitor compounds will generally reduce or inhibit statin-mediated myopathy by at least 10%, 20%, 30%, 40%, 50%, 60%, 70%, 80%, 90%, or more.

### Identification of New Compounds or Extracts

In general, compounds capable of decreasing the activity of atrogin-1 are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) Aldrich Chemical (Milwaukee, Wis.), and ChemBridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their molt-disrupting activity should be employed whenever possible.

When a crude extract is found to increase or decrease the biological activity or expression levels of an atrogin-1 polypeptide, or to bind to an atrogin-1 polypeptide, further fractionation of the positive lead extract is necessary to isolate chemical constituents responsible for the observed effect. Thus, the goal of the extraction, fractionation, and purification process is the careful characterization and identification of a chemical entity within the crude extract that decreases the biological activity of an atrogin-1 polypeptide. Methods of fractionation and purification of such heterogeneous extracts are known in the art. If desired, compounds shown to be useful as therapeutics for the treatment or prevention of an endothelial cell disorder or an angiogenic disorder are chemically modified according to methods known in the art.

### EXAMPLES

### Example 1

### Statin Toxicity in Human Muscle Biopsies is Associated with Atrogin-1 Expression

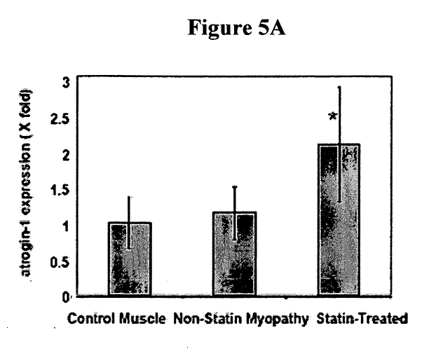

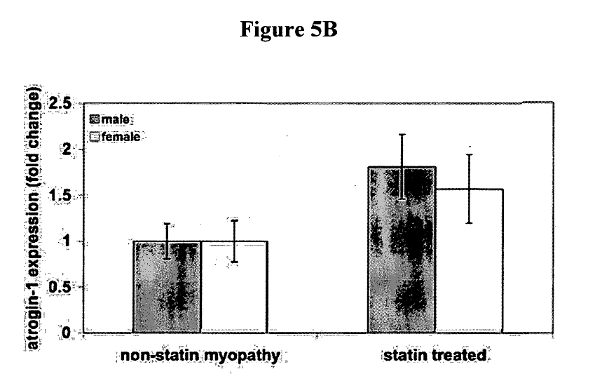

The atrogin-1 mRNA levels were measured in 17 human quadriceps muscle biopsies from five patients undergoing knee replacements (controls), from four patients with muscle pain but not being treated with statins, and from eight patients with muscle pain/damage concomitantly being treated with statins (the table below). As can be seen in FIG. 5A, atrogin-1 mRNA levels were significantly higher in the statin-treated muscle samples. Though the subjects were not strictly gender-matched, higher atrogin-1 mRNA levels were observed in both males and females who had been administered a statin (FIG. 5B).

TABLE 1
Human muscle biopsies from statin-treated and statin-untreated patients

| Group/Bx Number | | Age | Sex | Diagnosis | Clinical Data | Statin |
|---|---|---|---|---|---|---|
| Statin-treated | 3 | 51 | M | Statin-associated myopathy | Muscle pain and weakness, CK 47 | atorvastatin |
| | 4 | 75 | M | Statin-associated myopathy | Muscle pain and weakness, CK 73 | atorvastatin |
| | 5 | 41 | M | Statin-associated myopathy | Muscle pain and weakness, CK 62 | simvastatin |
| | 6 | 52 | M | Statin-associated myopathy | Muscle pain and weakness, CK 649 | simvastatin |
| | 40 | 60 | M | Statin-induced rhabdomyolysis | Weakness, CK 932 | lovastatin |
| | 41 | 82 | F | Statin-induced rhabdomyolysis | Weakness, CK 51,080 | simvastatin |
| | 43 | 87 | M | Statin-induced rhabdomyolysis | Weakness, CK 8,264 | simvastatin |
| | 45 | 62 | F | Statin-induced rhabdomyolysis | Weakness, CK 39,000 | simvastatin |
| Non-statin | 20 | 46 | M | Non-specific myopathy | Muscle pain and weakness, CK 635 | None |
| myopathy | 22 | 39 | F | Non-specific myopathy | Muscle pain and weakness | None |
| | 24 | 57 | F | Non-specific myopathy | Muscle pain and weakness | None |
| | 27 | 46 | F | Non-specific myopathy | Muscle pain and weakness, CK 2,300 | None |
| | 48 | 31 | M | Non-specific myopathy | Recurrent rhabdomyolysis | None |
| | 51 | 41 | M | Minor myopathic changes | Muscle pain and weakness, CK 800 | None |
| Control | 31 | 73 | F | osteoarthritis | Knee pain | None |
| | 33 | 61 | F | osteoarthritis | Knee pain | None |
| | 34 | 71 | F | osteoarthritis | Knee pain | None |
| | 35 | 67 | F | osteoarthritis | Knee pain | None |
| | 36 | 87 | F | osteoarthritis | Knee pain | None |

### Experimental Methods

Muscle biopsies: Muscle was obtained from three groups of patients whose characteristics are detailed in the above table. The statin-treated group included four subjects with statin-induced myopathy and 4 with statin-induced rhabdomyolysis, as commonly defined (Antons et al., Am. J. Med. 119:400-409, 2006). The non-statin myopathy group included four statin-naïve subjects with undefined myopathy. Both of these groups underwent percutaneous muscle biopsies of the vastus lateralis muscle using a Bergstrom needle. The control group included five statin-naïve subjects who volunteered muscle at the time of knee arthroplasty. Muscle from subjects in all three groups was snap frozen in liquid nitrogen for subsequent analysis. All subjects signed an IRB-approved consent form.

Quantitative PCR: Atrogin-1 mRNA levels were determined by real-time PCR using the Applied Biosystems® 7500 real-time PCR analyzer according to the method recently described by others (Okuno et al., Blood 100:4420-4426, 2002, and Yang et al., J. Cell Biochem. 94:1058-1067, 2005). Multiplexed amplification reactions were performed using 18S rRNA as an endogenous control (18S rRNA primers/VIC-labeled probe Applied Biosystems #4310893E) using the TaqMan One Step PCR Master Mix reagents Kit (#4309169, Applied Biosystems). The following settings were used: Stage 1 (reverse transcription): 48° C. for 30 min; Stage 2 (denaturation): 95° C. for 10 min; and Stage 3 (PCR): 95° C. for 15 sec and 60° C. for 60 sec for 40 cycles. The sequences of the forward, reverse, and double-labeled oligonucleotides for atrogin-1 were: forward 5′-CTT TCA ACA GAC TGG ACT TCT CGA-3′ (SEQ ID NO: 5); reverse 5′-CAG CTC CAA CAG CCT TAC TAC GT-3′ (SEQ ID NO: 6); and TaqMan® probe: 5′-FAM-TGC CAT CCT GGA TTC CAG AAG ATT CAA C-TAMRA-3′ (SEQ ID NO: 7). Fluorescence data were analyzed by SDS1.7 software (Applied Biosykems). The Ct (Threshold cycle) values for each reaction were transferred to a Microsoft Excel spreadsheet and calculation of relative gene expression was performed from this data according to published algorithms (TaqMan Cytokine Gene Expression Plate 1 protocol, Applied Biosystems). All RNA samples were analyzed in triplicate, with the mean value used in subsequent analyses.

### Example 2

### Lovastin Causes Atrogin-1 Induction in Cultured Myocytes

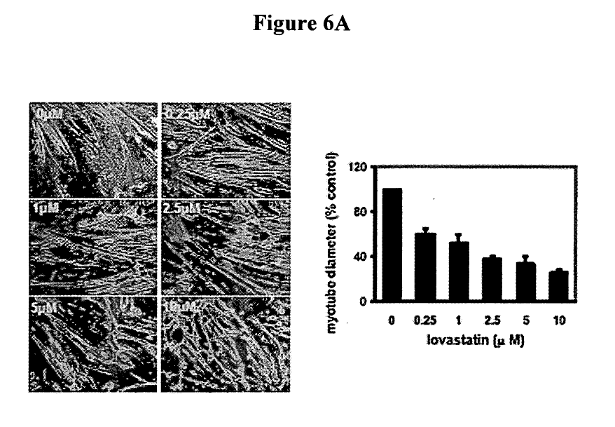

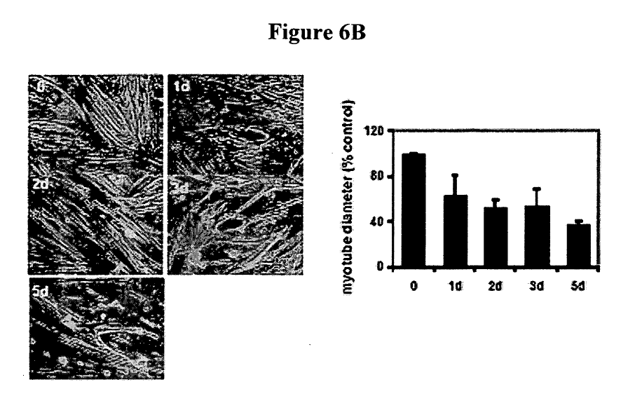

The effect of statins on muscle cells was studied by treating differentiated C2C12 mouse myocytes with various concentrations of lovastatin. Compared with control myotubes treated with an equal volume of vehicle, in the presence of increasing concentrations of lovastin, myotubes became progressively thinner and appeared to have more cytoplasmic vacuolation, changes in cell contour, and frank disruption or loss of myotubes (FIG. 6A). The reduction of myotube size was quantitated by measurement of myotube thickness. This effect was clearly visible at low lovastatin concentrations, in the range of those typically found in patients administered with this medication (Pan et al., J. Clin. Pharmacol. 30:797-801, 2002, and Hostein et al., Cancer Chemother. Pharmacol. 57:155-164, 2006). Morphological changes were visible in the myotube cultures after 24 hours of treatment, with almost complete loss of myotubes by 5 days (FIG. 6B). These detrimental effects were not unique to lovastatin, as similar results were observed when cultures were treated with a second statin, cerivastatin.

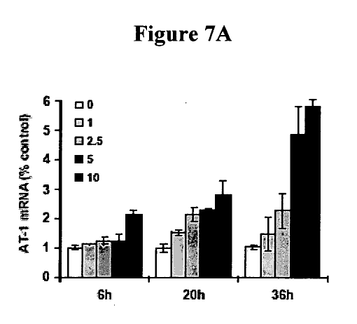

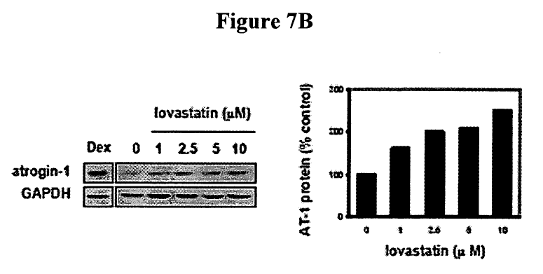

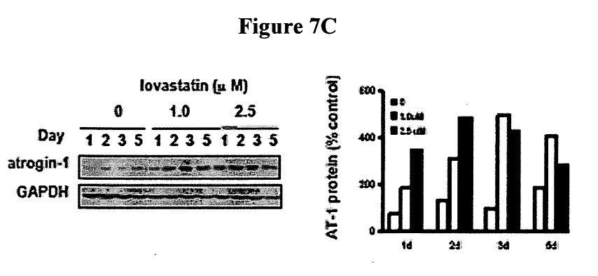

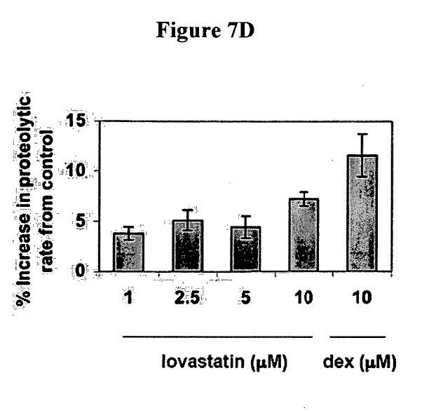

The effect of statins on expression of atrogin-1 in cultured mouse myocytes was also determined. Using real time PCR, atrogin-1 mRNA was found to be dramatically and rapidly induced by lovastatin in a time- and concentration-dependent manner (FIG. 7A). At the highest lovastatin concentration (10 μM), atrogin-1 mRNA was significantly increased at 6 hours, and induced as much as 6-fold by 36 hours of treatment. The atrogin-1 protein levels in lovastatin-treated mouse myocytes mirrored the observed increases in atrogin-1 mRNA (FIG. 7B). At low lovastatin concentration (1.0 μM) for 48 hours, atrogin-1 induction was about 1.5-fold, and at high concentration (10 μM), increased about 2.5-fold compared to non-treated control cells (FIG. 7C). This amount of atrogin-1 activation was similar to that found in myotubes atrophying due to dexamethasone treatment.

The rate of protein breakdown was also measured in the lovastatin-treated myotube cultures. As observed with dexamethasone, a known inducer of atrogin-1, treatment of myotube cultures with lovastatin led to a consistent 5-10% increase in the rate of bulk muscle proteolysis compared with control cultures (FIG. 7D).

### Experimental Methods

Cell Culture: Mouse myoblast cell line C2C12 was purchased from ATCC (ATCC, Manassas, Va.) and maintained in DMEM (Mediatech, Herndon, Va.) containing 10% fetal bovine serum (Hyclone, Logan, Utah) and penicillin (100U) and streptomycin (50 ug/ml; Invitrogen, Grand Island, N.Y.). When C2C12 cells reach to 90% confluence, medium was replaced with differentiation medium of DMEM supplemented with 2% horse serum (ATCC, Manassas, Va.) to induce myotube formation. Cells were used for experiments in 4-5 days after differentiation. Lovastatin (>98% purity) (mevinolin; Sigma, St. Louis, Mo.) was prepared as a 50 mM stock solution in DMSO as reagent vehicle, further diluted in DMSO, and added into the medium. The final volume DMSO in medium is not more than 0.125%, which there is not obvious cytotoxicity. Equal volume of reagent vehicle was used for all experiments and reagent vehicle only serviced as controls. Each experiment was performed at least three times.

Myotube Fiber Size: Size was quantified by measuring a total of 200 tube diameters as described by Sandri et al. (supra). Briefly, muscle fiber size from four random fields at 100 magnification was measured using IMAGE software (Scion, Frederick, Md.). All data were expressed in Mean±S.E.M. Comparisons were made by using the Student's T-test, with p<0.05 being considered statistically significant.

Quantitative PCR: Performed as described above.

Western Blotting Cultured cells after treatment were collected at specific times and solubilized in RIPA lysis buffer (50 mM Tris-HCl, pH 7.4; 150 mM NaCl, 1% NP-40, 0.5% sodium deoxycholate, 0.1% SDS (Boston Bioproducts, Boston Mass.), protease (Roche) and phosphatase (Sigma) inhibitor cocktail). Proteins were separated by SDS-PAGE, transferred to PVDF membranes and visualized by Western blotting using alkaline phosphatase-based CDP-star chemiluminescent detection according to manufacturer's protocol (Applied Biosystems, Bedford, Mass.).

Measurement of Proteolytic Rate: Differentiated C2C12 myotubes were incubated with ³H-tyrosine (5 μCi/mL media) for 20 hours to label cell proteins, and switched to medium containing 2 mM unlabeled tyrosine, vehicle, lovastatin, or dexamethasone for another 20 hours. After media replacement, an aliquot of medium was collected once per hour for 4 hours. The collected media was treated with TCA (10% w/v final concentration) to precipitate protein. Since the high concentration of unlabeled tyrosine in the media prevents re-incorporation of ³H-tyrosine into new protein, the radioactivity in the supernatant represent degraded protein from the pool of prelabeled, intracellular, long-lived proteins. Proteolytic rate was defined as the percentage of released radioactivity per hour calculated from the period of 20 hours to 24 hours of lovastatin or dexamethasone treatment, when ³H-tyrosine release is linear over time. These rates were compared with proteolytic rates in parallel cultures treated with vehicle alone. All measurements were done in triplicate and then independently repeated at least twice.

### Example 3

### Effect of Lovastin on Atrogin-1 (−/−) Primary Mouse Myocytes

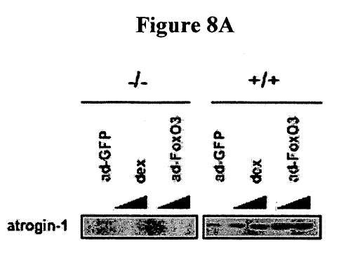

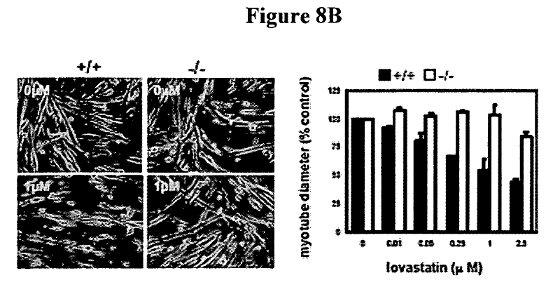

To test whether atrogin-1 expression is necessary for lovastatin-mediated muscle damage, primary myotubes from mice lacking atrogin-1 (Bodine et al., Science 294:1704-1708, 2001) were used. Primary myotubes derived from the atrogin-1 knockout mice were morphologically identical to cells from atrogin-1 wildtype control littermates (FIG. 8B). The atrogin-1 (−/−) myotubes indeed contained no atrogin-1 protein, and the control wildtype primary myotubes activated atrogin-1 expression after dexamethasone treatment or FoxO adenoviral infection (Sandri et al., supra) in a similar manner to the immortalized C2C12 myotubes (FIG. 8A). Lovastatin treatment caused very similar morphological changes in the atrogin-1-containing primary myotubes compared with C2C12 cells. However, primary myotubes lacking atrogin-1 had less damage than control cells at similar lovastatin concentrations (FIG. 8B). Little change in myotube size was noted was noted in the atrogin-1 (−/−) cells treated with 0.25 and 1.0 μM lovastatin, whereas in the control cultures, tube diameter decreased by as much as 50% after two days of exposure to the drug. The results clearly demonstrate that atrogin-1 is an important factor in lovastatin-induced myotube damage.

### Experimental Methods

In addition to methods described above, the following methods were used:

Cell Culture: Primary mouse myoblasts from atrogin-1 null mice (Regeneron, Tarrytown N.Y.) were isolated as follows: muscle was removed from the hind limbs of two-week old mice. After treatment with 0.1% collagenase D and Dispase II (Roche, Indianapolis, Ind.), the isolated cells were plated on collagen (Type I, Roche, Indianapolis, Ind.)-coated dishes. Myoblasts were subsequently enriched and cultured in F-10 nutrient medium with 20% fetal calf serum and 2.5 ng/ml bFGF (Invitrogen, Grand Island, N.Y.). Myotubes were induced in differentiation medium. All media contained 1× Primocin (InvivoGen, San Diego, Calif.). The cultures were maintained at 37° C., under 5% and 8% CO₂ humidified air atmosphere for myoblasts and myotubes, respectively. Cultures were ready to use in assays on day 2 in differentiation medium when the myotubes had formed and were contracting.

### Example 4

### Lovastin Promotes Damage of Muscle Fibers in Zebrafish Embryos

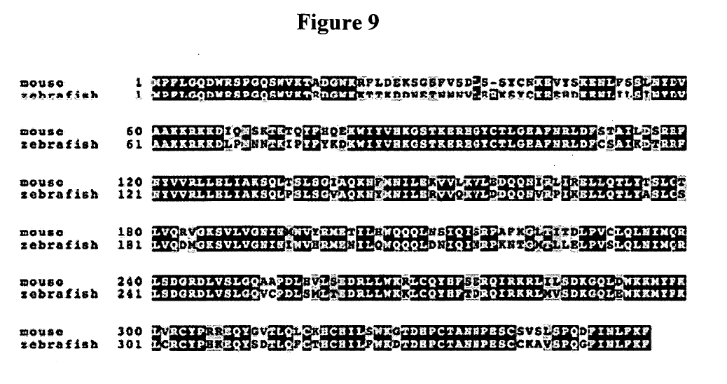

An in vivo model to study effects of lovastatin administration on muscle development was created. Zebrafish embryos were used as an in vivo model as: 1) whole body muscle fibers can be stained in zebrafish embryos at 48 hours post-fertilization (hpf) (Birely et al., Devel. Biol. 280:162-176, 2005); 2) zebrafish are amenable to rapid genetic manipulations, and 3) mouse and zebrafish atrogin-1 are 75% identical and 86% similar at the amino acid level (FIG. 9).

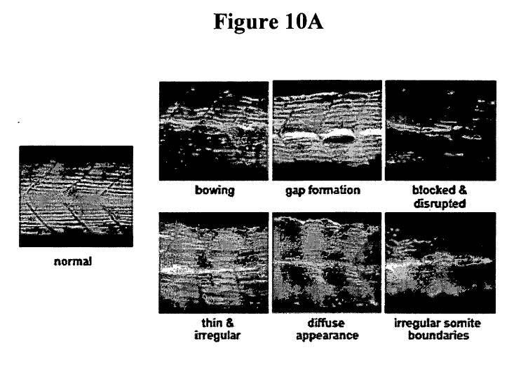

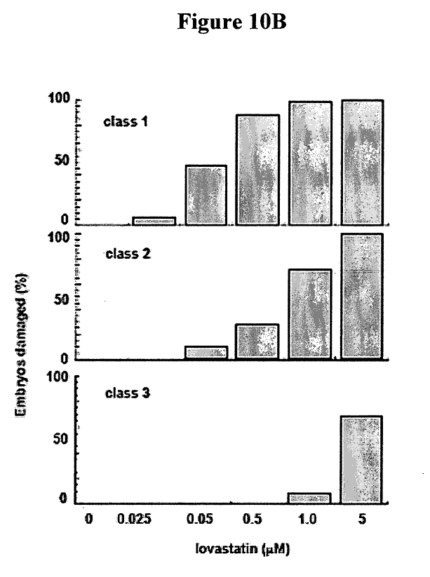

Zebrafish embryos were treated with lovastatin from 20 hpf to 32 hpf at different concentrations (0-5 μM). As in mammalian muscle cell culture, lovastatin led to clear dose-dependent muscle phenotypes, demonstrated by longitudinal muscle fiber staining with an antibody to myosin heavy chain (FIG. 10A). Muscle damage at low lovastatin concentration (0.025-0.05 μM) was evidenced by bowing, gap formation, and fiber disruption (class 1 changes). At higher lovastatin concentration (0.05-0.5 μM), fiber damage was more severe. Fiber thining and attenuation of staining with the MHC antibody was frequently observed (class 2 changes). At maximal lovastatin concentration (1.0-5.0 μM), damage beyond the muscle was observed, with the development of irregular somite boundries (class 3 changes). Using this classification, we found that class 3 changes were observed in over 60% of embryos subjected to 5 μM lovastatin, however, more than 50% of embryos treated with concentrations ten-fold lower (i.e., 0.05 μM) still demonstrated milder, class 1 defects (FIG. 10B).

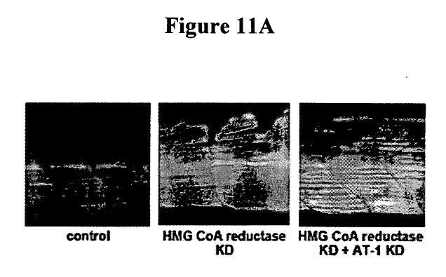

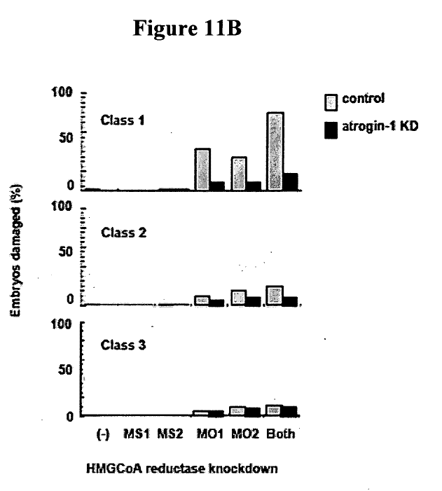

To confirm that lovastatin's effect on zebrafish muscle was mediated via inhibition of HMG CoA reductase rather than another off-target effect, the zebrafish HMG CoA reductase gene (z-HMG CoA reductase) in zebrafish embryos was knocked down using both missense and antisense morpholino oligonucleotides targeting the ATG region of the gene (ATG morpholino). Depletion of z-HMG CoA reductase showed similar effects as lovastatin treatment in zebrafish muscle fibers (FIGS. 11A and 11B).

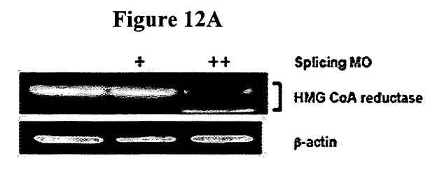

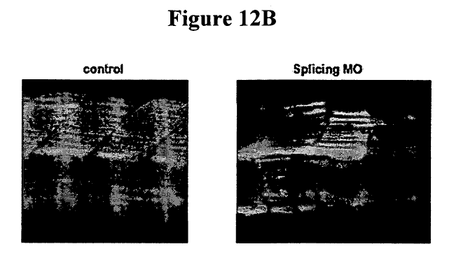

To further document the role of z-HMG CoA reductase in maintaining zebrafish muscle fiber morphology, active z-HMG CoA reductase was also depleted by creating a splicing morpholino oligonucleotide against the common splice site of both splice variants of the HMG CoA reductase gene in zebrafish (FIG. 12A). Use of this morpholino oligonucleotide in zebrafish embryos resulted in an abnormal muscle fiber structure similar to the z-HMG CoA reductase knockdown using the ATG morpholino and wildtype embryos treated with lovastatin (FIG. 12B).

### Experimental Methods

Zebrafish lines and maintenance: Adult zebrafish (Danio rerio) were maintained as described under standard laboratory conditions at 28.5° C. in a 14 h light/10 h dark cycle (Westerfield, “The zebrafish book: a guide for the laboratory use of zebrafish Danio (Brachydanio) rerio,” Institute of Neuroscience, University of Oregon, Eugene, Oreg., 1993). Developmental stages were determined by embryo morphology and hours post fertilization (hpf) (Kimmel et al., Dev. Dyn. 203:253-310, 1995). To examine the effects of statin, the embryos at 20-24 hpf were immersed in the embryonic water (500 μM NaCl, 170 μM KCl, 330 μM CaCl2, and 330 μM MgSO4) at a concentration of 0.005-10 μM lovastatin (Mevinolin; Sigma, St. Louis, Mo.) including 0.003% 1-phenyl-2-thiourea (Sigma) to inhibit pigmentation in 24 well plate. After 32 hpf, the embryos were fixed by 4% paraformaldehyde in PBS.

Antibody staining: Whole zebrafish staining: Zebrafish embryos were fixed by 4% paraformaldehyde in PBS overnight. After fixation, the embryos were washed by PBS, stored for at least 1 h at −20° C. in methanol, and permeabilized for 30 min at −20° C. in acetone. Embryos were incubated with blocking buffer (1% BSA, 0.1% Tween-20 in PBS), and incubated with diluted primary antibody, anti-slow twitch myosin F59 (1: 200; Developmental Studies Hybridoma Bank (DSHB), Department of Biological Sciences, University of Iowa, Iowa City, Iowa 52242) (Crow and Stockdale, Dev. Biol. 118:333-342, 1986; and Devoto et al., Development 122:3371-3380, 1996) in blocking solution for overnight at 4° C. Staining was detected by using goat anti-mouse TRITC secondary antibody (1:200; Southern Biotechnology Associates, Inc.) in blocking solution for 4 h at RT (Birely et al., Dev. Biol. 280:162-176, 2005). Cross-sectional staining: Embryos were fixed overnight in 4% paraformaldehyde (PFA) and cryoprotected by the overnight incubation with increasing concentrations of sucrose (up to 30%). Samples were embedded in OCT compound and then equilibrated to −80° C. Sections (10 μm thick) were collected on SuperFrost/Plus slides and dried. Sections were rehydrated in PBS and blocked for 1 h in blocking buffer (1% BSA, 0.1% Tween in PBS). Sections were incubated overnight at 4° C. with primary antibody diluted in blocking buffer. Sections were stained with antibody as above.

Western blotting: Zebrafish embryos were homogenized in SDS sample buffer (30 embryos/30 μl of sample buffer) with microfuge pestle until the lysate became uniform in consistency and there was no longer stringy inside. The lysate was boiled for 5 minutes and centrifuged supernatant was processed for Western blotting (Hanai et al., J. Cell Biol. 158:529-539, 2002).

### Example 5

### Atrogin-1 Knockdown Prevents Statin-Induced and HMG-CoA Reductase Knockdown-Induced Muscle Injury in Zebrafish Embryos

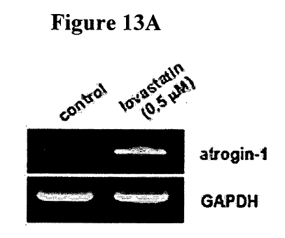

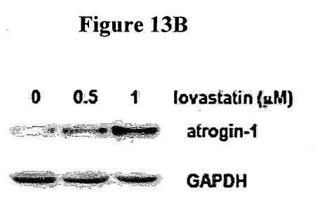

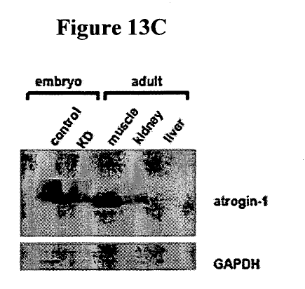

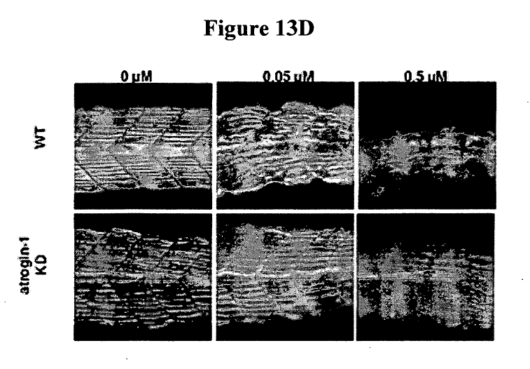

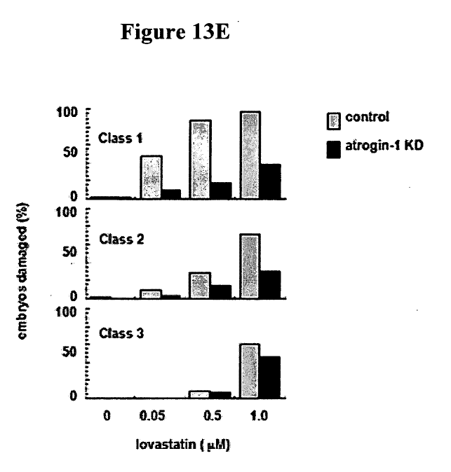

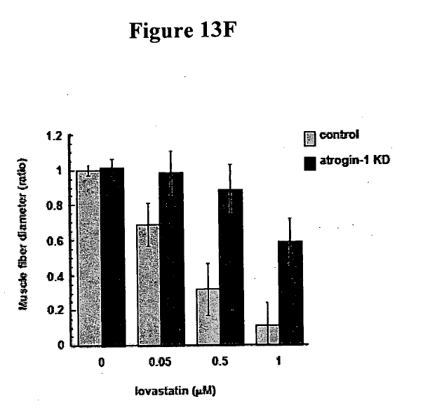

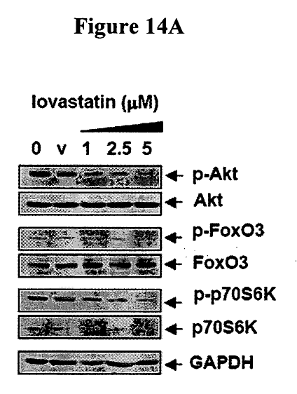

Since atrogin-1 is strongly induced in mammalian muscle cultures following lovastatin administration (supra), experiments were performed to determine if atrogin-1 was induced in lovastatin-treated zebrafish embryos. As in mammalian cells, the zebrafish homologue of atrogin-1 was clearly and dose-dependently elevated upon lovastatin treatment in the fish at both the mRNA and protein level (FIGS. 13A and 13B, respectively). To determine if atrogin-1 is required for the morphological effects of lovastatin on zebrafish muscle, an antisense morpholino oligonucleotide against the atrogin-1 gene was produced. Injection of this morpholino oligonucleotide into zebrafish embryos effectively knocked down endogenous atrogin-1 gene expression (FIG. 13C). No significant gross or histological abnormalities were observed in z-atrogin-1-depleted embryos (FIG. 13D). Wildtype embryos and z-atrogin-1-depleted embryos were then treated with lovastatin (0-1.0 μM). A significant rescue of the muscle damage phenotype was observed in the z-atrogin-1-depleted embryos (compare FIGS. 13D, 13E, and 13F). The muscle defects caused by the z-HMG-CoA reductase knockdown were also significantly reduced in the z-atrogin-1 knockdown (FIG. 13B).

For these experiments, the same methods as described in Example 4 were used.

### Example 6

### FoxO3a Activity is Suppressed Following Lovastatin Treatment

Suppression of IGF-1/PI3K/Akt signaling leading to dephosphorylation, nuclear translocation and activation of FoxO3 are key events in atrogin-1 induction. The effects of statin administration on this signaling pathway were examined in muscle cell culture and in zebrafish.

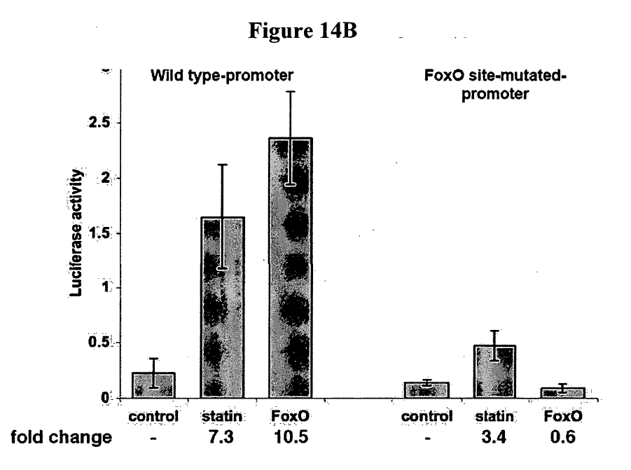

Treatment of C2C12 myotubes with lovastatin led to a dose-dependent reduction of phosphorylated signaling intermediates including phosphor-Akt, phosphor-FoxO3, and phosphor-p70S6K (FIG. 14A). The effect of lovastatin on FoxO-dependent activation of the atrogin-1 promoter in zebrafish embryos was also examined. Embryos were injected with a proximal fragment of the atrogin-1 promoter linked to luciferase or the same fragment with the FoxO site mutated (Sandri et al., Cell 117:399-412, 2004). Lovastatin (0.5 μM) stimulated the reporter luciferase activity more than 7-fold, while stimulating the FoxO-less reporter only 3-fold (FIG. 14B). The studies suggest that statin-induced atrogin-1 transcription is mediated by FoxO dephosphorylation and activation. Since lovastatin treatment still lead to a small amount of luciferase activity even in the absence of FoxO binding sites in the atrogin-1 promoter-reporter, additional signaling pathways may also be important in mediating the effects of statins on atrogin-1 expression in muscle.

### Example 7

### PGC-1α is an Inhibitor of Atrogin-1

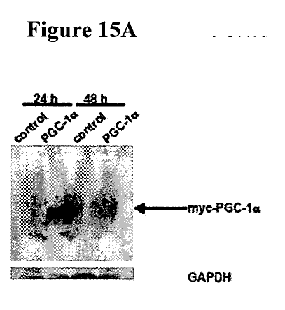

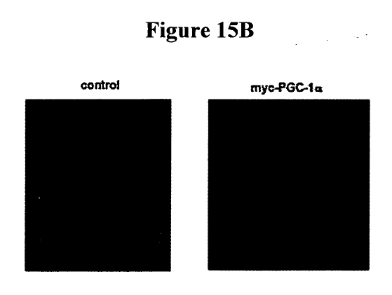

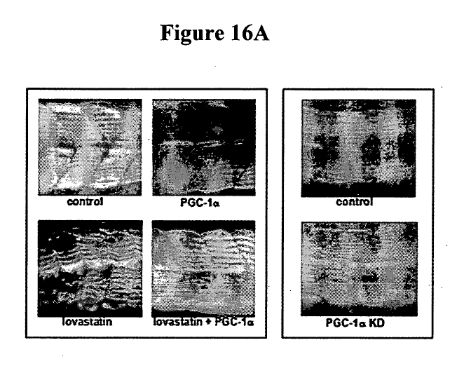

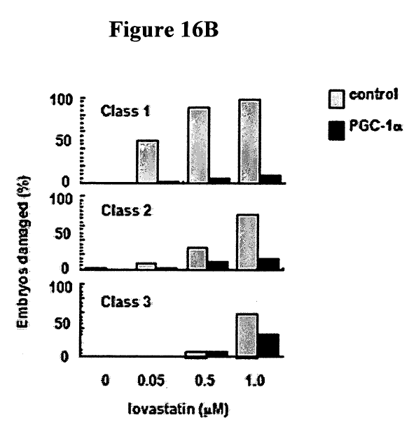

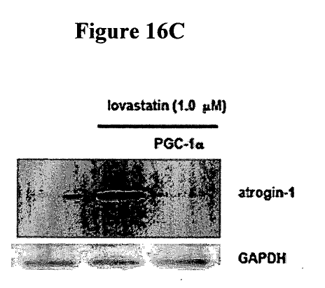

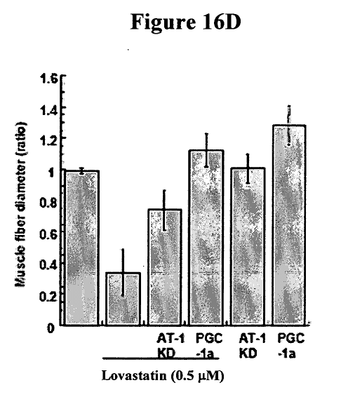

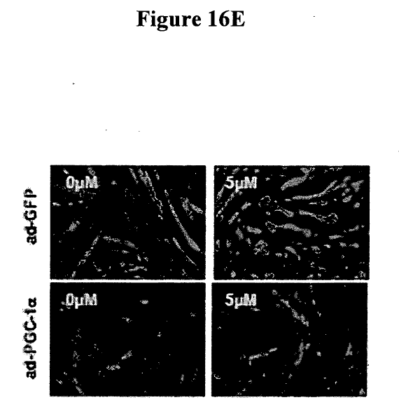

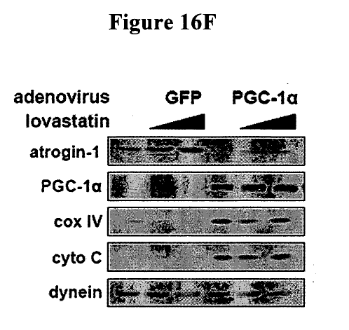

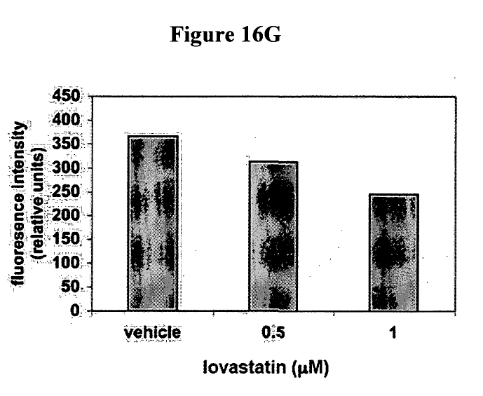

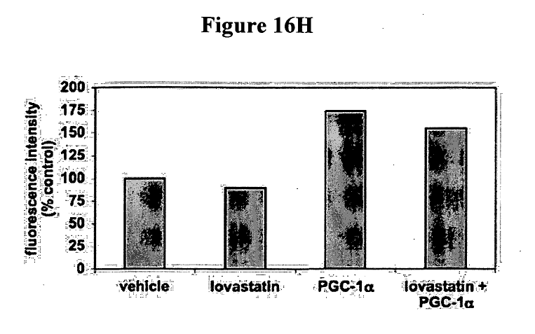

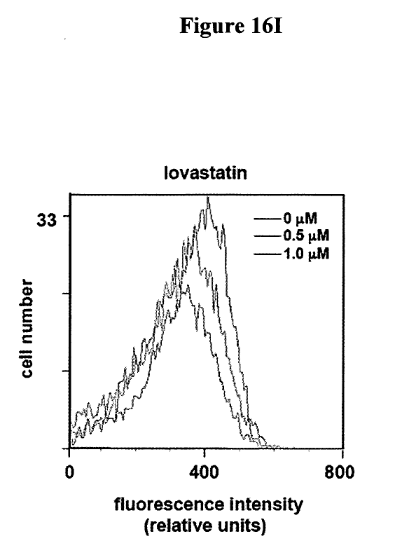

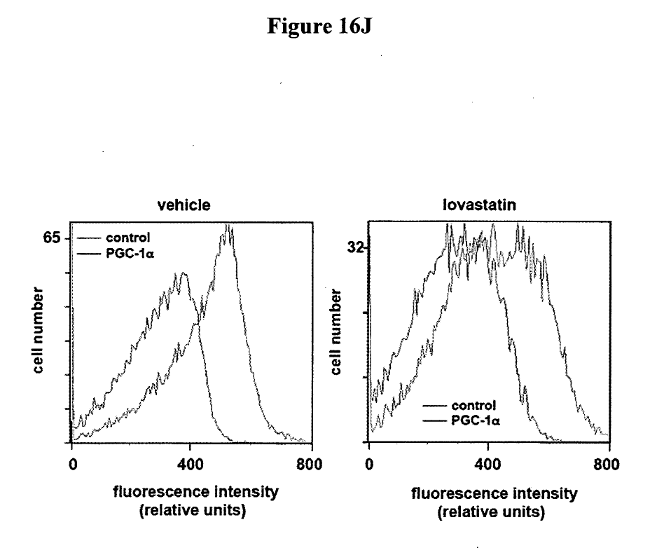

We have discovered that atrogin-1 is strongly activated following lovastatin treatment. Since PGC-1α expression prevents atrogin-1 induction (Sandri et al., Cell 117:399-412, 2006), the effect of PGC-1α expression on statin-induced muscle injury in zebrafish was examined. Injection of cDNA bearing PGC-1α into zebrafish embryos led to robust protein expression (FIG. 15A) and dramatically prevented muscle damage by lovastatin (FIG. 15B, FIG. 16A, and FIG. 16B). Expression of PGC-1α in zebrafish embryos completely inhibited the lovastatin-induced expression of zebrafish atrogin-1 protein (FIG. 16C) and protected against fiber size reduction (FIG. 16D). Expression of PGC-1α in cultured muscle cells using adenoviral vectors also protected from lovastatin toxicity (FIG. 16E). In the presence of PGC-1α overexpression, 5 μM lovastatin caused almost no change in myotube integrity or size. Likewise, PGC-1α overexpression completely suppressed atrogin-1 induction in these cultures and increased the expression of other mitochondrial proteins (FIG. 16F).

To further monitor mitochondrial function during lovastatin treatments, cells from untreated embryos and embryos exposed to lovastatin (0.5 or 1.0 μM) were stained with a fluorescent dye taken up by functional mitochondria (MitoTracker) (Poot et al., J. Histochem. Cytochem. 44:1363-1372, 1996). The fluorescence intensity of zebrafish cells following treatment with lovastatin was shifted to the left signifying a decrease in mitochondrial function or content in these cells (FIG. 16G and FIG. 16I). Interestingly, cells overexpressing PGC-1α following treatment with lovastatin, were significantly more fluorescent and were less effected by lovastatin treatment (FIG. 16H and FIG. 16I).

Taken together, these experiments show that PGC-1α expression protects against muscle damage and that PGC-1α acts as an inhibitor of atrogin-1 expression.

### Experimental Methods

For these experiments, the same methods as described in Example 4 were used. In addition, methods for mitochondria staining were used and are detailed below.

Mitochondrial staining and FACS analysis: Embryos were treated with lovastatin (0, 0.5, 1.0 μM at 20-32 hpf) or treated with the combination of lovastatin (0.5 μM) following PGC-1α (or vehicle) cDNA injection (100 pg/embryo at the one-cell stage). 1-Phenyl-2-thiourea (0.003%; Sigma) was added at 20 hpf. After phenotypes were observed, 100 embryos from each condition were decholionated by protease and homogenized for 3-5 minutes in 0.9× phosphate buffered saline (PBS)/10% fetal bovine serum (FBS), then centrifuged at 3000 rpm for 5 minutes, digested with trypsin/EDTA and dispersed at room temperature. After adding 1 mL of 0.9×PBS/10% FBS, the dispersed cells were filtered (100 μM pore size) and washed twice with 0.9×PBS/10% FBS. The cells were incubated in 100 nM MitoTracker Red CMXRos (Invitrogen) in 0.9×PBS/10% FBS for 15 minutes in the dark. The cells were washed twice in 0.9×PBS/10% FBS and subjected to fluorescence cell-assisting sorting (FACS) analysis. EPICS XL (Beckman Coulter) was used for the fluorescence detection (absorption wavelength: 578 nm; emission wavelength: 599 nm) and data was analyzed with Expo32ADC software. Ten thousand cells were counted for each treatment condition.

### Example 8

### Generation of Anti-Atrogin-1 Polyclonal Antibody

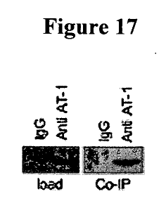

To produce a recombinant atrogin-1 peptide, pGC2-atrogin-1 was cut with EcoRI and Stu1 to liberate 650 by of atrogin-1. This fragment was inserted into pET28b (Novagen) previously digested with NotI and blunt-ended with Klenow fragment followed by digestion with EcoRI. The resulting plasmid contained a 110-amino acid NH₂-terminal fragment of the atrogin-1 gene behind a His₆ tag and an isopropylthiogalactoside (IPTG)-inducible promoter. This fragment was purified from E. coli BL21 (DE3) under denaturing conditions using a Ni-NTA Agarose affinity column (Qiagen) according to the manufacturer's instructions. The purified protein was dialyzed in phosphate buffered saline (PBS) and subsequently used to generate a rabbit polyclonal IgG antibody (anti-atrogin-1 IgG). Anti-atrogin-1 IgG was affinity purified from the IgG according to the procedures of Harlow and Lane (Antibodies: A Laboratory Manual, New York, Cold Spring Laboratory, 1988), using an Affigel-10 matrix (Bio-Rad Laboratories) onto which the purified atrogin-1 fragment was bound. The prepared anti-atrogin-1 IgG has the ability to recognize both denatured atrogin-1 (e.g., denatured by sodium dodecyl sulfate) and native atrogin-1 (e.g., in immunoprecipitate complexes; FIG. 17).

### Experimental Methods

Methods of antibody production from a purified protein are known in the art (e.g., the methods described in Kohler and Milstein, Nature, 256: 495-497, 1975; Kohler and Milstein, Eur. J. Immunol., 6, 511-519, 1976; and Campbell, “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985).

### Example 9

### Atrogin-1 Changes Intracellular Localization During Atrophy

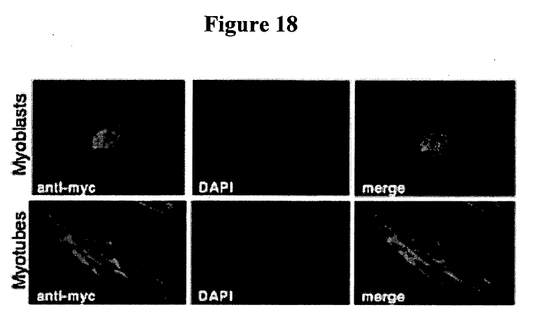

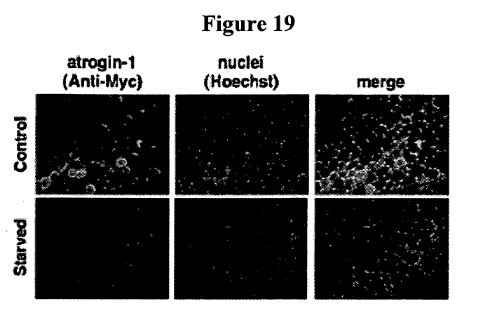

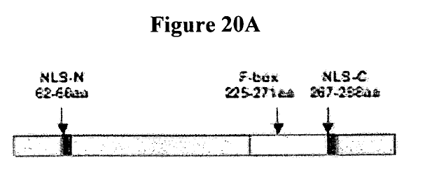

In studies to investigate the cellular localization of atrogin-1, it was discovered that an adenoviral vector expressing atrogin-1 led to nuclear localization in undifferentiated myocytes, but the same vector led to a cytoplasmic atrogin-1 distribution in differentiated myotubes (FIG. 18). Using the technique of electroporation of plasmid DNA into living mouse muscles (Sandri et al., supra), myc₆-atrogin-1 was introduced into mouse tibialis anterior muscle. In control, non-atrophying muscle, atrogin-1 was distributed in both cytoplasmic and nuclear locations, however, when muscles containing the atrogin-1 construct were starved, a condition which promotes muscle atrophy, atrogin-1 shifted to a predominantly nuclear location (FIG. 19).

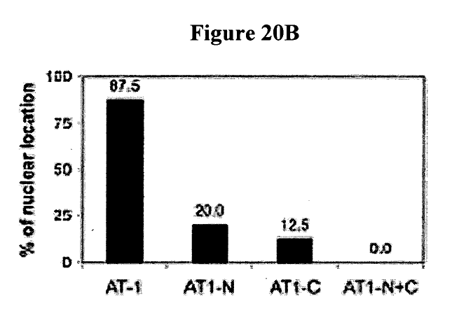

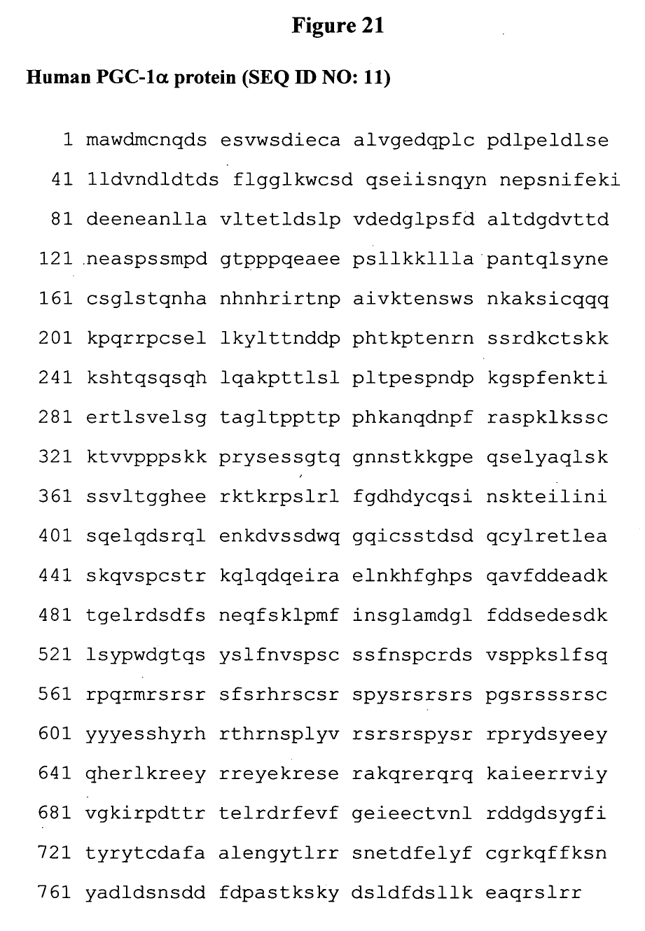

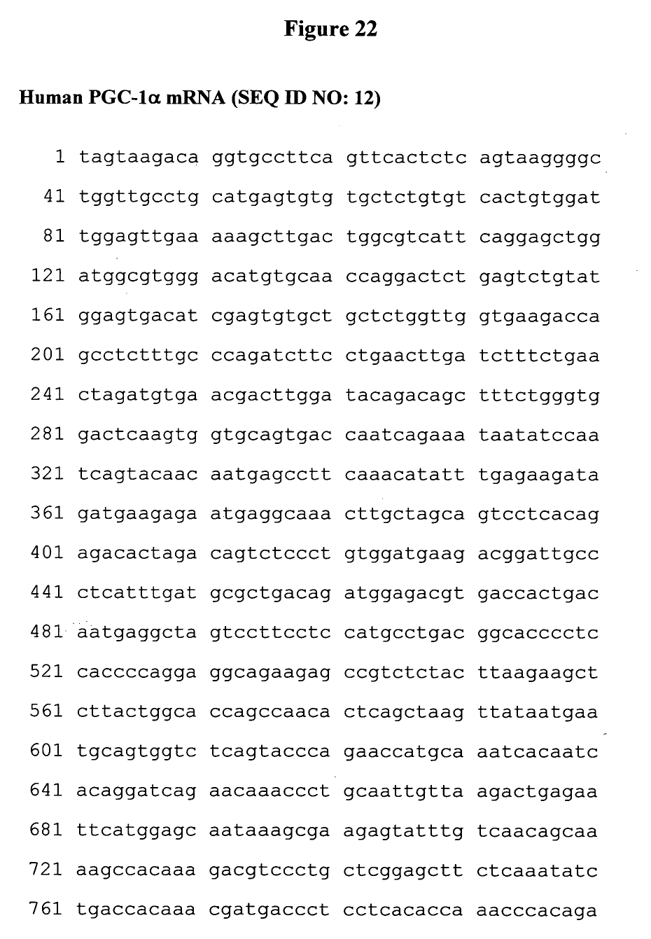

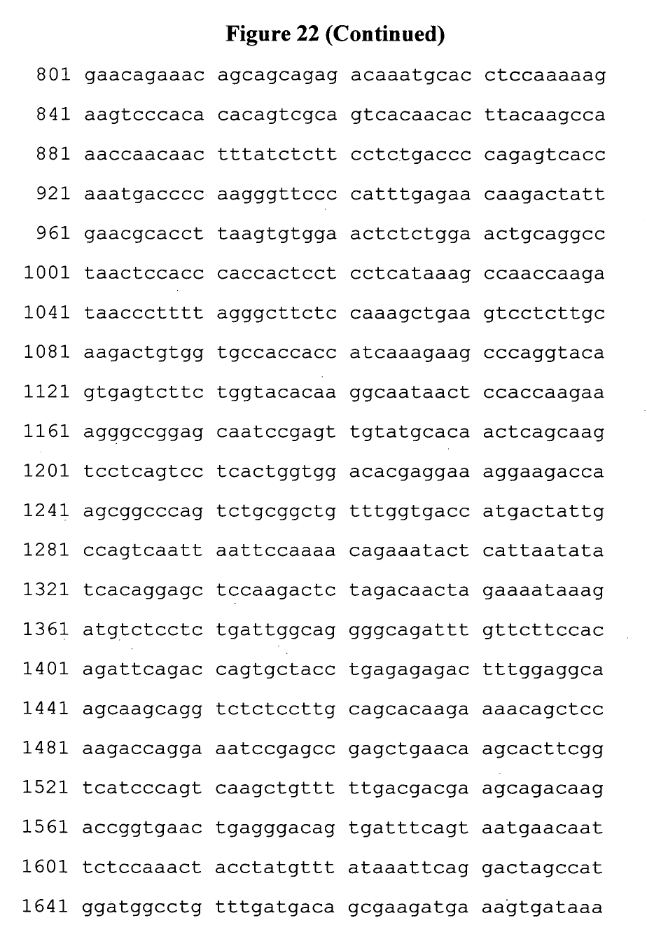

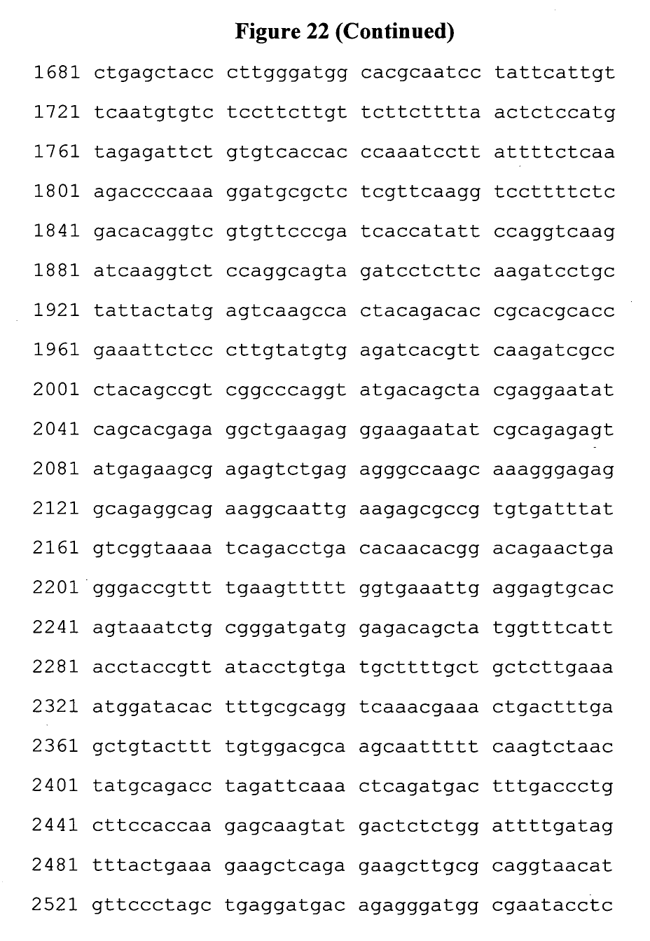

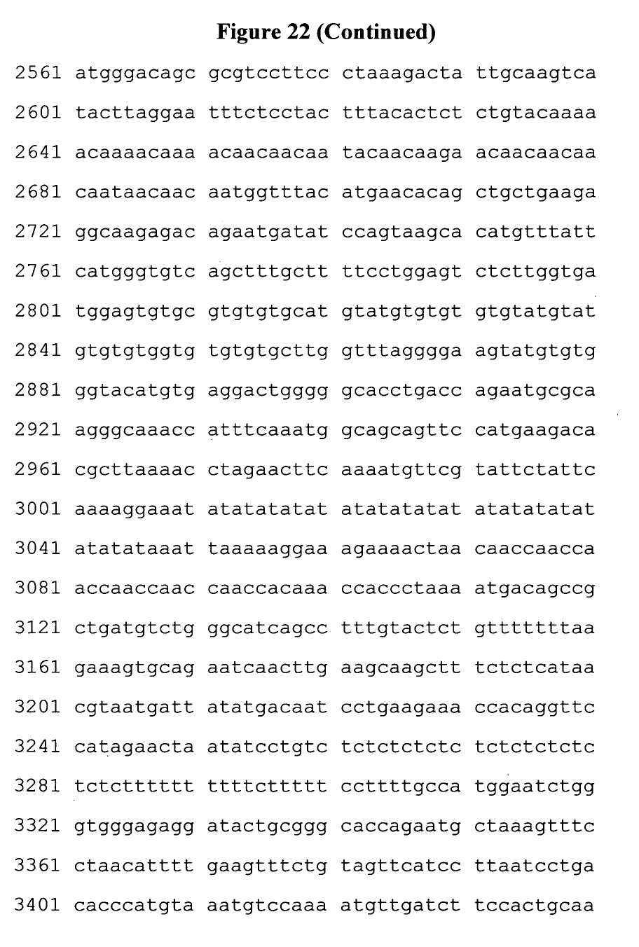

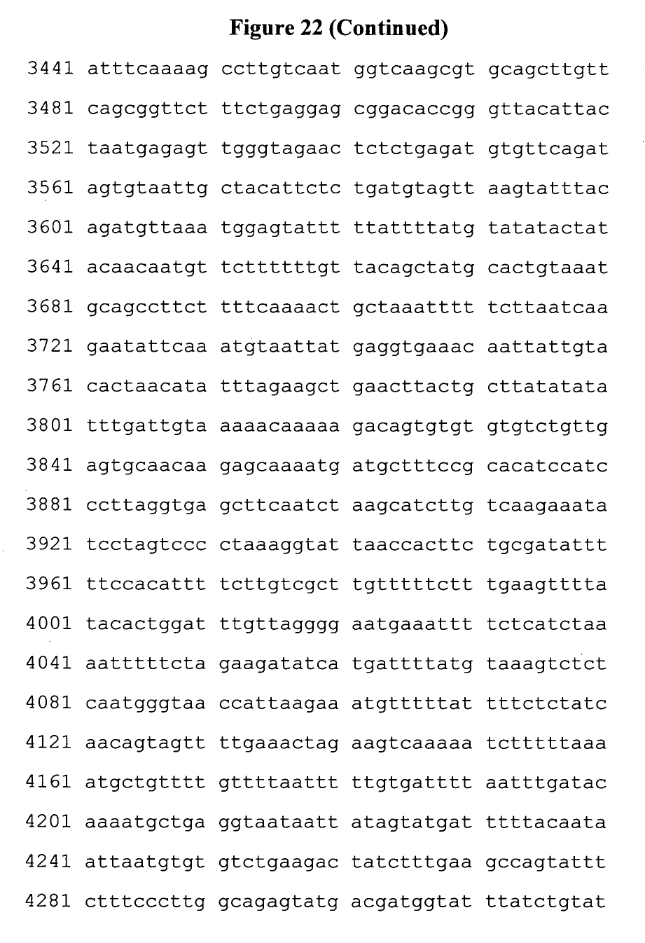

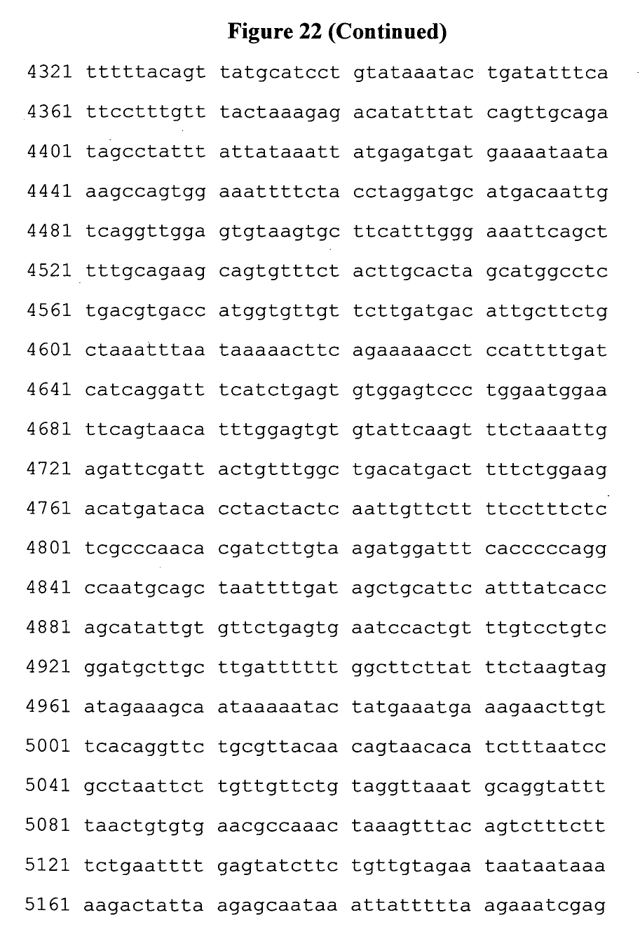

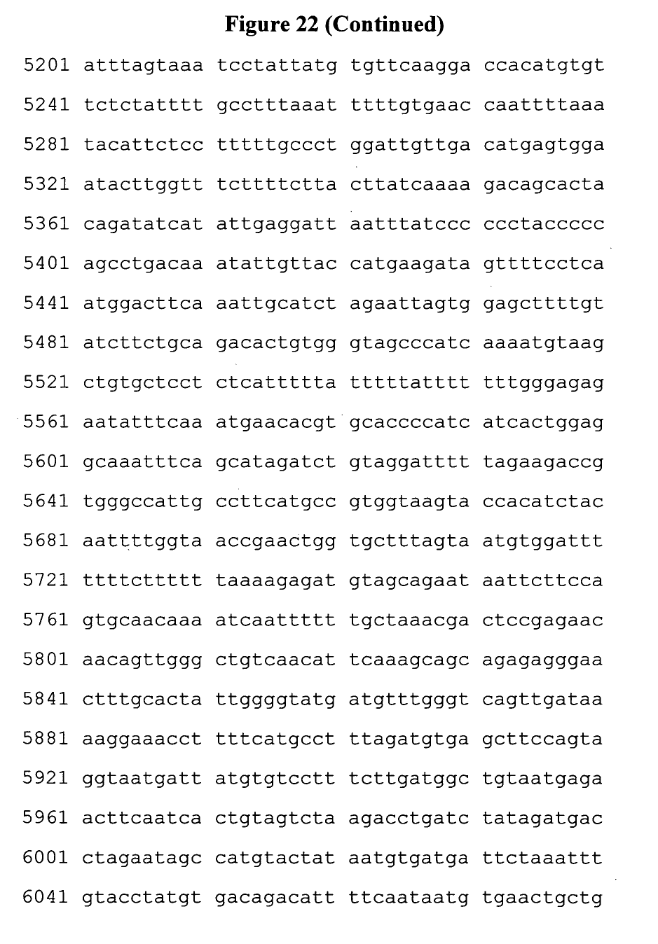

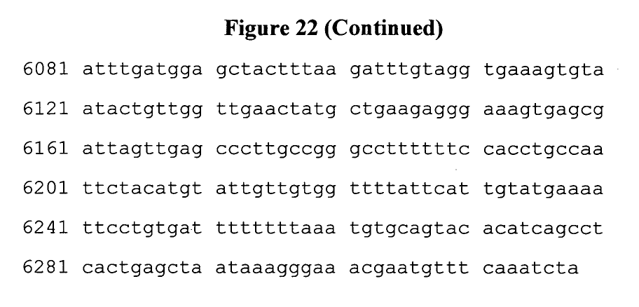

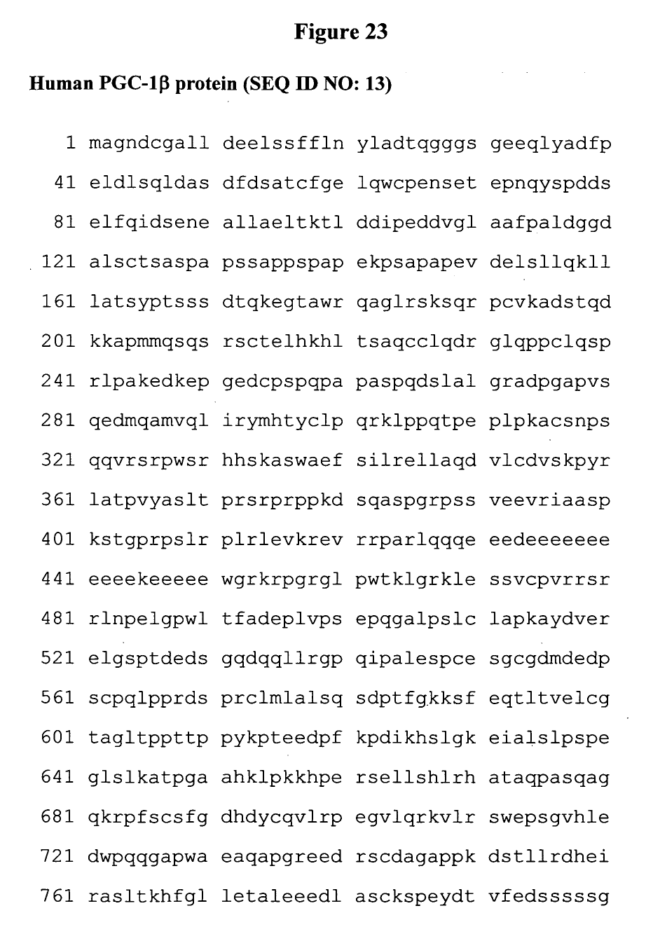

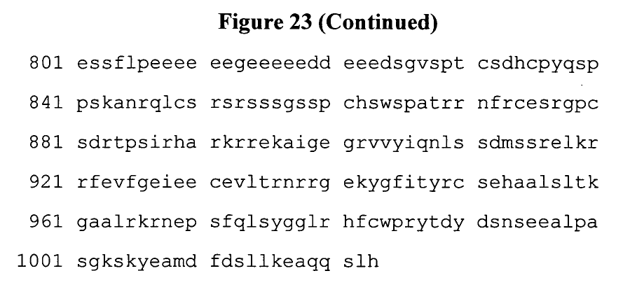

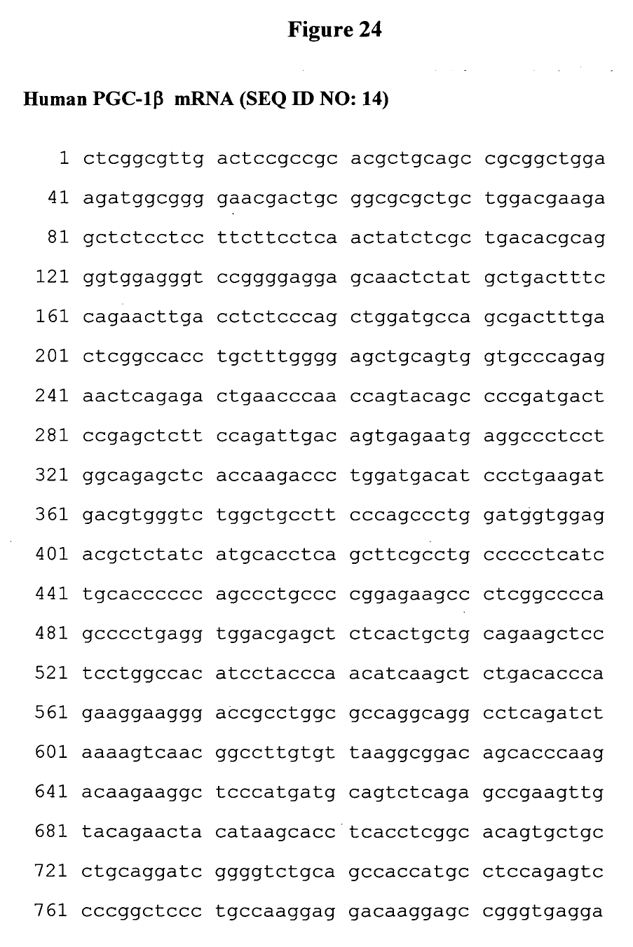

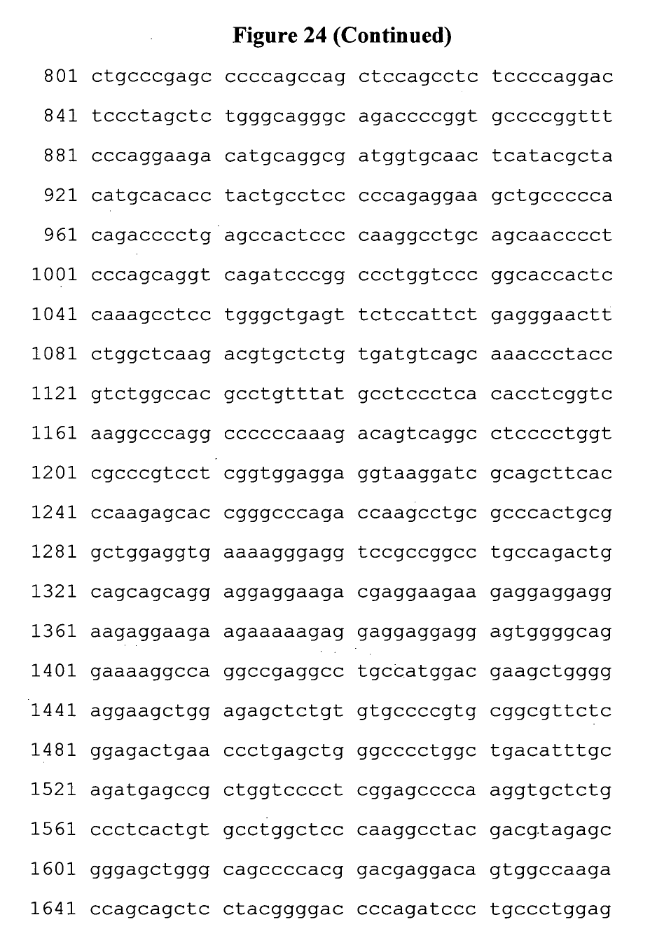

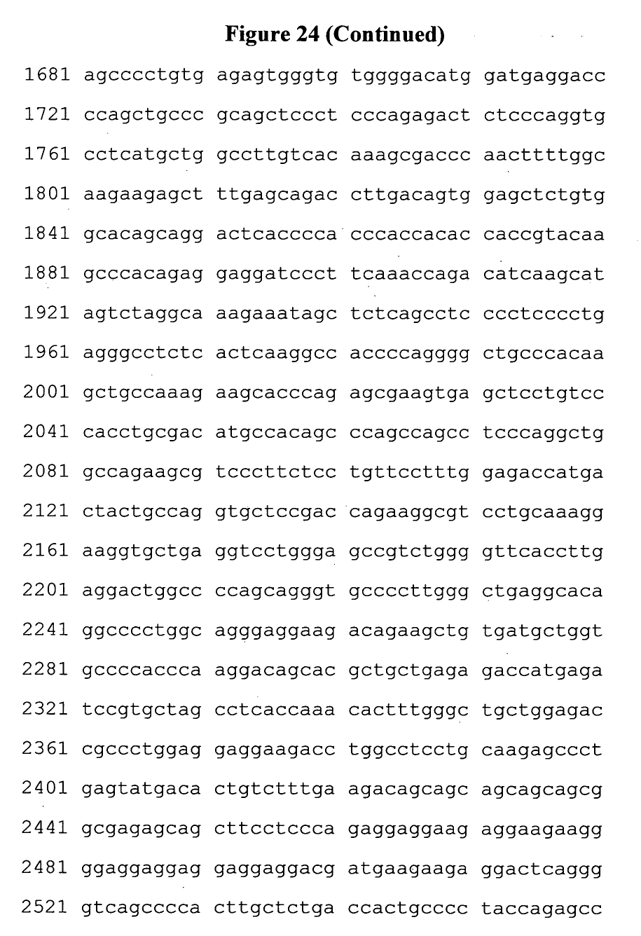

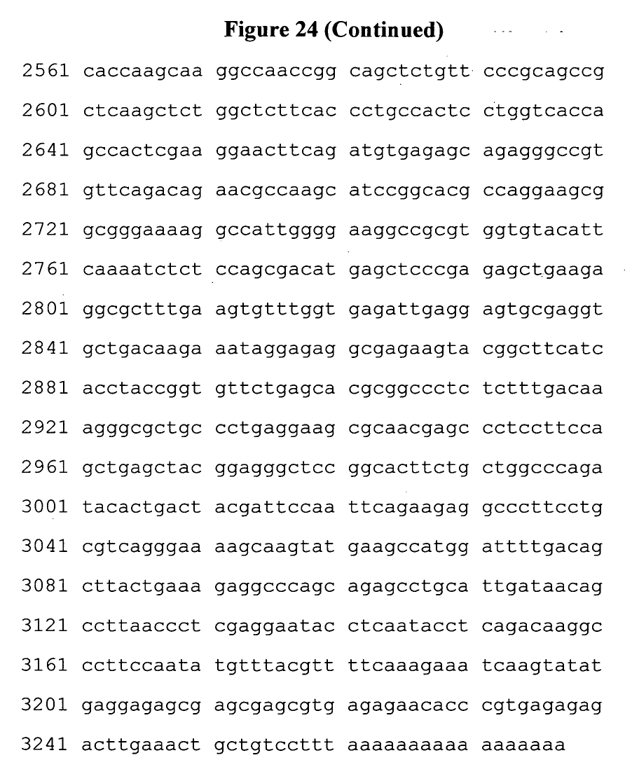

To further investigate the importance of nuclear localization to the ability of atrogin-1 to mediate muscle atrophy, mutations of the putative nuclear localization sequences (i.e., amino acids 62-66 and amino acids 267-288) were made in a myc-tagged form of the atrogin-1 gene. Three constructs were made to mutate the N-terminal, C-terminal, and both the N- and C-terminal putative atrogin-1 nuclear localization sequences (FIG. 20A). Plasmids bearing these constructs were transfected into 293T cells and expression of atrogin-1 was measured by anti-myc immunofluorescence. Both the single region deletions retained some of their nuclear localization (10-20% of wildtype), while the double mutation was rendered completely cytoplasmic (FIG. 20B). The experiments indicate the nuclear localization may be required for the role of atrogin-1 in muscle atrophy. Therefore, molecules which block the nuclear translocation of atrogin-1 may be effective inhibitors of atrogin-1 activity in the cell.

### Experimental Methods

Methods of immunofluorescence microscopy and transfection are known to those skilled in the art.

### Other Embodiments

All publications, patents, and patent applications cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art. Other embodiments are within the claims.

What is claimed is:

1. A method of treating or preventing a statin-mediated myopathy in a subject, said method comprising administering to said subject a therapeutically effective amount of an atrogin-1 inhibitor compound in an amount and for a time sufficient to treat or prevent said statin-mediated myopathy in said subject.

2. The method of claim 1, wherein said atrogin-1 inhibitor compound reduces or inhibits the expression levels or biological activity of an atrogin-1 protein or nucleic acid.

3. The method of claim 2, wherein said atrogin-1 inhibitor compound is PGC-1α polypeptide or PGC-1α polypeptide.

4-5. (canceled)

6. The method of claim 2, wherein said biological activity of said atrogin-1 polypeptide is ubiquitin ligase activity, substrate binding activity, or nuclear translocation.

7. (canceled)

8. The method of claim 1, wherein said atrogin-1 inhibitor compound specifically binds the ubiquitin ligase domain, the substrate-binding domain of atrogin-1, or the N- or C-terminal nuclear localization sequence of atrogin-1.

9-10. (canceled)

11. The method of claim 1, wherein said atrogin-1 inhibitor compound is an antibody or antigen-binding fragment thereof that specifically binds atrogin-1.

12-13. (canceled)

14. The method of claim 1, wherein said atrogin-1 inhibitor compound reduces or inhibits the expression levels of an atrogin-1 nucleic acid molecule.

15-16. (canceled)

17. The method of claim 14, wherein said atrogin-1 inhibitor compound is a morpholino oligomer that is complementary to at least a portion of an atrogin-1 nucleic acid molecule.

18. The method of claim 17, wherein said morpholino oligomer comprises a sequence substantially identical to SEQ ID NO: 8 or SEQ ID NO: 9.

19. The method of claim 14, wherein said atrogin-1 inhibitor compound is a small RNA having at least one strand that comprises a nucleic acid sequence substantially identical to at least a portion of an atrogin-1 nucleic acid molecule, or a complementary sequence thereof.

20-24. (canceled)

25. The method of claim 1, wherein said subject has been treated with a statin.

26. (canceled)

27. The method of claim 25, wherein said subject is still being treated with said statin.

28-31. (canceled)

32. The method of claim 1, wherein said subject will be treated with a statin compound and said atrogin-1 inhibitor compound is administered prior to administering said statin compound.

33. (canceled)

34. A composition comprising an atrogin-1 inhibitor compound that reduces or inhibits the expression or biological activity of atrogin-1, wherein said compound is formulated for the treatment or prevention of a statin-mediated myopathy.

35-46. (canceled)

47. The composition of claim 34, wherein said atrogin-1 inhibitor compound reduces or inhibits the expression levels of an atrogin-1 nucleic acid molecule.

48-49. (canceled)

50. The composition of claim 47, wherein said atrogin-1 inhibitor compound is a morpholino oligomer that is complementary to at least a portion of an atrogin-1 nucleic acid molecule.

51. The composition of claim 50, wherein said morpholino oligomer comprises a sequence substantially identical to SEQ ID NO: 8 or SEQ ID NO: 9.

52. The composition of claim 47, wherein said atrogin-1 inhibitor compound is a small RNA having at least one strand that comprises at least a portion of an atrogin-1 nucleic acid molecule, or a complementary sequence thereof.

53-54. (canceled)

55. A kit comprising:
i) a statin;
ii) an atrogin-1 inhibitor compound; and
iii) instructions for administration of said statin and said atrogin-1 inhibitor compound for the treatment of a statin-induced myopathy.

56-57. (canceled)

58. A method of diagnosing a subject as having or having a propensity to develop a statin-mediated myopathy, said method comprising measuring the level of an atrogin-1 polypeptide, atrogin-1 nucleic acid, or fragments thereof, in a sample from said subject relative to a reference sample or level, wherein an alteration in said subject levels relative to said reference sample or level is diagnostic of a statin-mediated myopathy or a propensity to develop a statin-mediated myopathy in said subject.

59. The method of claim 58, wherein said reference sample or level is a normal reference sample or level and said alteration is an increase.

60-61. (canceled)

62. A method of diagnosing a subject as having or having a propensity to develop a statin-mediated myopathy, said method comprising measuring the level of an antibody, or a fragment thereof, that specifically binds atrogin-1 in a blood or serum sample from said subject relative to a reference level, wherein an alteration in said subject levels compared to said reference level is diagnostic of a statin-mediated myopathy or a propensity to develop a statin-mediated myopathy in said subject.

63. The method of claim 62, wherein said reference is a normal reference level and said alteration is an increase.

64. (canceled)

65. A method of monitoring a statin-mediated myopathy or a propensity to develop a statin-mediated myopathy in a subject, said method comprising measuring the level of an atrogin-1 polypeptide, nucleic acid, atrogin-1 specific antibody, or fragments thereof in a sample from said subject, and comparing said level to a reference sample or level, wherein an alteration in said level is an indicator of a change in the propensity to develop a statin-mediated myopathy, or a change in a statin-mediated myopathy of the subject.

66-85. (canceled)

86. A method of treating a biological sample from a subject having a statin-induced myopathy or a propensity to develop a statin-induced myopathy comprising the steps of:
(i) obtaining a biological sample from a subject having a statin-induced myopathy or a propensity to develop a statin-induced myopathy; and
(ii) treating said biological sample ex vivo with a therapeutically effective amount of an atrogin-1 inhibitor compound.

87-89. (canceled)
